(19) United States
(12) Patent Application Publication
(10) Pub. No.: US 2023/0218638 A1
(43) Pub. Date: Jul. 13, 2023

(54) NEUROACTIVE COMPOUNDS AND METHODS OF USE THEREOF

(71) Applicant: SAGE THERAPEUTICS, INC., Cambridge, MA (US)

(72) Inventors: Michael C. Quirk, Acton, MA (US); James J. Doherty, Bedford, MA (US); Gabriel Martinez Botella, Wayland, MA (US)

(21) Appl. No.: 17/947,844

(22) Filed: Sep. 19, 2022

Related U.S. Application Data
(63) Continuation of application No. 15/517,886, filed on Apr. 7, 2017, now abandoned.
(60) Provisional application No. 62/060,932, filed on Oct. 7, 2014.

(51) Int. Cl. A61K 31/575; A61K 45/06; A61K 31/00; A61K 45/00

(52) U.S. Cl. CPC A61K 31/575; A61K 45/06; A61K 31/00; A61K 45/00

(57) ABSTRACT

Methods for treating a subject suffering from a sterol synthesis disorder or a sterol deficiency disorder, e.g., Smith-Lemli-Opitz syndrome, the method comprising administering to the subject an effective amount of an NMDA receptor modulating compound, are provided.

### RELATED APPLICATIONS

This application claims priority to U.S. Provisional Application No. 62/060,932, filed Oct. 7, 2014, the entire contents of which are incorporated herein by reference.

### BACKGROUND OF THE INVENTION

NMDA receptors are highly expressed in the CNS and are involved in excitatory synaptic transmission. Activating these receptors contributes to synaptic plasticity in some circumstances and excitotoxicity in others. These receptors are ligand-gated ion channels that admit Ca2+ after binding of the neurotransmitters glutamate and glycine, and are fundamental to excitatory neurotransmission and normal CNS function. NMDA receptors are heteromeric complexes comprised of NR1, NR2, and/or NR3 subunits and possess distinct recognition sites for exogenous and endogenous ligands. These recognition sites include binding sites for glycine, and glutamate modulators. Positive modulators may be useful as therapeutic agents with potential clinical uses as cognitive enhancers and in the treatment of psychiatric disorders in which glutamatergic transmission is reduced or defective (see, e.g., Horak et al., J. of Neuroscience, 2004, 24(46), 10318-10325). In contrast, negative modulators may be useful as therapeutic agents with potential clinical uses in the treatment of psychiatric disorders in which glutamatergic transmission is pathologically increased (e.g., treatment resistant depression).

NMDA modulator compounds, e.g., neuroactive steroids such as pregnenolone sulfate (PS) have been shown to exert direct modulatory effects on several types of neurotransmitter receptors, such as GABA_{A}, glycine, AMPA, kainate, and NMDA receptors. NMDA receptors are positively modulated by PS; however, the degree of modulation varies considerably, e.g., depending upon the subunit composition of the receptor.

New and improved neuroactive compounds are needed that modulate brain excitability for the prevention and treatment of CNS-related conditions. The methods described herein are directed toward this end.

### SUMMARY OF THE INVENTION

Thus, in one aspect, described herein are methods of treating a sterol synthesis disorder such as SLOS or a sterol deficiency disorder. The methods of treatment can include treating a subject by administering to the subject an NMDA receptor modulating compound. Exemplary compounds are described herein.

In one aspect, described herein is a method of treating a subject suffering from a sterol synthesis disorder (e.g., disorder of cholesterol biosynthesis; disorder characterized by a significant disruption of sterol biosynthesis) or a sterol deficiency disorder (e.g., abnormal levels of a sterol described herein; e.g., at least 1, e.g., at least 2 standard deviations below normal sterol levels), comprising administering to the subject an effective amount of an NMDA receptor modulating compound or pharmaceutically acceptable salt thereof.

As used herein, “normal sterol level” varies by age, and is defined, as described in as Björkhem et al., J. of Lipid Res. 2001, 42: 366-371; for example, within 2 standard deviations of the values provided in Table 2 (e.g., within 2 standard deviations, within 1.5 standard deviations, or within 1 standard deviation). Normal and abnormal sterol levels (e.g., 24(S)-hydroxycholesterol and 27(S)-hydroxycholesterol) for example in various age groups, have been reported in e.g., Björkhem et al., J. of Lipid Res. 2001, 42: 366-371; Bretillon et al., J. Lipid Res. 2000, 41: 840-845; Björkhem et al., J. Lipid Res. 1998, 39: 1594-1600; Lutjohann et al., Proc. Natl. Acad. Sci. USA 1996, 93: 9799-9804, the contents of each of which are incorporated herein by reference.

In some embodiments, the subject suffers from a sterol synthesis disorder and a 24(S)-hydroxycholesterol deficiency disorder.

In some embodiments, the sterol deficiency disorder is characterized by the presence of 24(S)-hydroxycholesterol in the plasma of the subject at significantly reduced levels (e.g., at least 1 or 2 standard deviations below) compared with the plasma of a subject not suffering from a sterol deficiency disorder).

In some embodiments, the metabolic processing of 24(S)-hydroxycholesterol is low as compared with a subject not suffering from the disorder.

In some embodiments, the compound is 24(S)-hydroxycholesterol. In some embodiments, the compound is not a product of nature. In some embodiments, the sterol is 24(S)-hydroxycholesterol, 25-hydroxycholesterol, or 27(S)-hydroxycholesterol.

In some embodiments, the sterol disorder is selected from: Smith-Lemli-Opitz syndrome; Conradi-Hunermann syndrome; Greenberg dysplasia; Desmosterolosis; Cerebrotendinous Xanthomatosis (CTX); Mevalonate Kinase Deficiency Syndromes (MKD); SC4MOL gene mutation (SMO Deficiency); lathosterolosis; X-linked dominant chondrodysplasia puncata; CHILD syndrome or CK-syndrome; autism spectrum disorder; Niemann-Pick disease; and disorders of dolichol synthesis or metabolism. In some embodiments, the sterol disorder is Smith-Lemli-Opitz syndrome.

In some embodiments, the compound has an EC₅₀ of 101.1M or less (e.g., 5 μM, 1 μM, 500 nM, 350 nM, 250 nM, 100 nM, 50 nM, 10 nM or less).

In some embodiments, the compound is present at an effective plasma concentration of 10 to 800 ng/mL of plasma (e.g., 10 to 600 ng/mL, 10 to 500 ng/mL, 25 to 500 ng/mL, 40 to 500 ng/mL, 25 to 350 ng/mL). In some embodiments, the compound is present at an effective plasma concentration of at least 10 ng/mL of plasma (e.g., at least 15 ng/mL, 20 ng/mL, 25 ng/mL, 30 ng/mL, 30 ng/mL, 35 ng/mL, 40 ng/mL, 45 ng/mL, 50 ng/mL, 55 ng/mL).

In some embodiments, the compound is a NMDA receptor modulator (e.g., positive modulator, negative modulator).

In some embodiments, the compound is a compound of Formula (I), (II-a), (II-b), (III), (IV), (V), (VI), (VII), (VIII), (IX-A), (IX-B), (X), (XI-A), or (XI-B) In some embodiments, the compound is a compound of Formula (I).

In some embodiments, the administration to the subject normalizes concentrations of oxysterols in circulation relative to a subject not administered with the compound or administered with a placebo.

In some embodiments, the administration to the subject elevates levels of cholesterol in tissues and plasma relative to a subject not administered with the compound or administered with a placebo.

In some embodiments, the subject is an infant. In some embodiments, the subject is less than 21 years old (e.g., less than 18, 15, 13, 12, 10, 8, 6, 4, 3, 2, or 1 year old).

In some embodiments, the method further comprises administration of an additional therapy. In some embodiments, the additional therapy is dietary cholesterol therapy (e.g., cholesterol supplementation, statin treatment (e.g., 3-hydroxy-3-methylglutaryl coenzyme A reductase inhibitors (e.g., HMG CoA reductase inhibitors), e.g., simvastatin), bile acid supplementation or downstream hormone supplementation, medical therapies, or surgical interventions; antioxidants; gene therapy.

In one aspect, described herein is a dosage form comprising a compound of Formula (I), (II-a), (II-b), (III), (IV), (V), (VI), (VII), (VIII), (IX-A), (IX-B), (X), (XI-A), or (XI-B) configured for administration in a subject, wherein the subject is a child. In some embodiments, the dosage form is a liquid suspension, sprinkle, meltaway, sublingual, or injectable. In some embodiments, the dosage form is a solid dosage form. In some embodiments, the solid dosage form is a tablet, capsule, or pill.

### DETAILED DESCRIPTION OF CERTAIN EMBODIMENTS OF THE INVENTION

### Diseases and Disorders

Described herein are methods of treating a sterol synthesis disorder. Exemplary disorders are described herein. The methods include administering to a subject, e.g., a subject suffering from a sterol synthesis disorder such as SLOS, a NMDA receptor modulating compound. Exemplary compounds are described herein. In some embodiments the compounds is 24(S) hydroxyl cholesterol. In some embodiments, the compound is a compound of a formula described herein such as a compound of Formula (I), (II-a), (II-b), (III), (IV), (V), (VI), (VII), (VIII), (IX-A), (IX-B), (X), (XI-A), or (XI-B).

### Sterol Synthesis Disorders

In one aspect, described herein are methods for treating a sterol synthesis disorder. Cholesterol has an essential rule in growth and development. It is a membrane lipid and a precursor to many molecules that play important roles in cellular growth and differentiation, protein glycosylation, and signaling pathways. Biosynthesis of cholesterol involves a number of enzymes and intermediates. Disorders resulting from a deficiency in any of the enzymes involved in cholesterol biosynthesis lead to the accumulation of intermediates and imbalance in biomolecules, resulting in disorders including congenital skeletal malformations, dysmorphic facial features, psychomotor retardation, and failure to thrive. In an embodiment, a sterol synthesis disorder or symptom of a sterol synthesis disorder can be treated by administering to a subject suffering from a sterol synthesis disorder a compound described herein, such as a NMDA receptor modulating compound as described herein. Additional disorders are described below.

### Smith-Lemli-Opitz Syndrome

In one aspect, described herein are methods for treating Smith-Lemli-Opitz Syndrome (or SLOS, or 7-dehydrocholesterol reductase deficiency). SLOS is an inborn error of cholesterol synthesis. In addition to microcephaly, moderate to severe intellectual disability, sensory hypersensitivity, stereotyped behaviors, dysmorphic facial features, and syndactyly of the second/third toes, a feature of the disease is reduced cerebrosterol (24(S)-hydroxycholesterol) levels. SLOS is an autosomal recessive genetic condition resulting from deficiency in the final enzyme of the cholesterol synthesis pathway, and causes low or low-normal plasma cholesterol levels and increased 7- and 8-dehydrocholesterol (DHC; 7DHC and 8DHC) levels. Common therapies currently used include dietary cholesterol supplementation, treatment with 3-hydroxy-3-methylglutaryl coenzyme A reductase inhibitors (HMG CoA reductase inhibitors, also known as statins), and treatment with agents that enhance cholesterol production and/or accretion; and to decrease the accumulation of 7DHC and 8DHC, the potentially toxic precursors of cholesterol.

### Desmosterolosis

Desmosterolosis is a deficiency in desmosterol reductase and has a similar phenotype to SLOS. In one aspect, described herein are methods for treating desmosterolosis.

### Sitosterolemia

Sitosterolemia is a rare autosomal recessive disorder caused by mutations in two ATP-binding cassette (ABC) transporter genes (ABCG5 and ABCG8). Sitosterolemia enhances the absorption of plant sterols and cholesterol from the intestines. Patients typically present with tendon and tuberous xanthomas and premature coronary artery disease. In one aspect, described herein are methods for treating sitosterolemia.

### Cerebrotendinous Xanthomatosis (CTX)

In one aspect, described herein are methods for treating cerebrotendinous xanthomatosis (also referred to as cerebral cholesterosis, or Van Bogaert-Scherer-Epstein syndrome). CTX can be caused by a mutation in the CYP27A1 gene, which produces the sterol 27-hydroxylase enzyme. Sterol 27-hydroxylase metabolizes cholesterol into bile acids (e.g., chenodeoxycholic acid) that are important in the absorption of fats in the intestine. Enzyme dysfunction can lead to cholesterol accumulation in tissues. CTX is characterized by childhood diarrhea, cataracts, tendon xanthomas, reduced mental capability and abnormal movements in adults.

### Mevalonate Kinase Deficiency Syndromes (MKD)

Mevalonate Kinase Deficiency (also referred to as mevalonic aciduria (a more severe form of MKD), or Hyper IgD Syndrome (HIDS, or hyperimmunoglobulinemia D) with period fever syndrome (a more benign form of MKD)) causes an accumulation of mevalonic acid in the urine as a result of insufficient activity of mevalonate kinase. MKD can result in developmental delay, hypotonia, anemia, hepatosplenomegaly, dysmorphic features, mental retardation, and overall failure to thrive. Mevalonic aciduria is characterized by delayed physical and mental development, failure to thrive, recurrent episodes of fever with vomiting and diarrhea, enlarged liver, spleen and lymph nodes, microcephaly (small head size), cataract, low muscle tone, short statute, distinctfacial features, ataxia, and anemia. HIDS is characterized by recurrent episodes of fever associated with swollen lymph nodes, joint pain, gastrointestinal issues and skin rash. In one aspect, described herein are methods for treating MKD.

### SC4MOL Gene Mutation (SMO Deficiency)

SC4MOL gene deficiency is a genetic disorder in the cholesterol biosynthesis pathway (e.g., mutations in the SC4MOL gene encoding a novel sterol oxidase). SC$MOL deficiency is characterized by the accumulation of dimethyl and monomethyl sterols that can be detected in blood, skin flakes or primary skin fibroblasts. In one aspect, described herein are methods for treating SMO deficiency.

### Niemann-Pick Disease

Niemann-Pick disease is a lysosomal storage disease resulting from a genetic mutation that affects metabolism. Niemann-Pick disease leads to abnormal accumulation of cholesterol and other fatty substances (lipids) due to an inability of the body to transport the substances. The accumulation damages the affected areas.

### Autism

In one aspect, described herein are methods for treating autism spectrum disorder or autism. Autism spectrum disorder (ASD) and autism refer to a group of complex disorders of brain development. Autism is typically characterized by difficulties in social interaction, for example in verbal and nonverbal communication. Repetitive behaviors are also often seen in individuals having autism. Autism can be associated with intellectual disability, difficulties in motor coordination and attention and physical health issues, e.g., sleep and gastrointestinal disturbances. Individuals having autism can also excel in visual skills, music, math and art. Autism can refer to autistic disorder, childhood disintegrative disorder, pervasive developmental disorder-not otherwise specified (PDD-NOS), and Asperger syndrome. Autism also refers to monogenetic causes of autism such as synaptophathy's, e.g., Rett syndrome, Fragile X syndrome, Angelman syndrome.

Disorders Associated with Phenylketonuria

In one aspect, described herein are methods for treating disorders associated with phenylketonuria (e.g., cognitive disorders). Phenylketonuria can lead to hypochesterolemia and lowered vitamin D status. Total and low-density cholesterols and 25-hydroxy vitamin D have been found to be decreased in subjects suffering from phenylketonuria as compared with subjects not suffering from phenylketonuria (Clin. Chim. Acta 2013, 416: 54-59). 24S-hydroxycholesterol and 27S-hydroxycholesterol and 7α-hydroxycholesterol (e.g., representing peripheral and hepatic cholesterol elimination, respectively) have been shown to be significantly decreased in subjects suffering from phenylketonuria, while 7β-hydroxycholesterol (e.g., reflecting oxidative stress) was increased significantly in subjects suffering from phenylketonuria. Changes in the levels of 24S—OHC and 7β-hydroxycholesterol correlate with phenylalanine level, and 27S-hydroxycholesterol levels may correlate with the 25-hydroxy vitamin D level in subjects suffering from phenylketonuria.

### Compounds

Described herein are methods of treating a sterol synthesis disorder such as SLOS, by administering to a subject an NMDA receptor modulating compound. Exemplary compounds are described herein below. In some embodiments, the compound is a negative modulator (e.g., a negative allosteric modulator). In some embodiments, the compound is a positive modulator (e.g., a positive allosteric modulator). In some embodiments, the compound is an allosteric modulator.

Exemplary compounds include a compound of Formula (I):

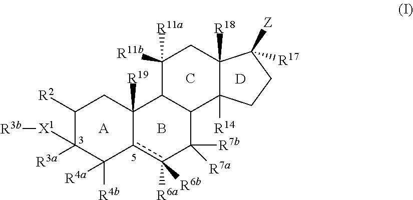

or a pharmaceutically acceptable salt, solvate, prodrug, stereoisomer, tautomer, isotopic variant, or N-oxide thereof, or a combination thereof;
wherein:

Z is a group of the formula (i), (ii), (iii), (iv), or (v):

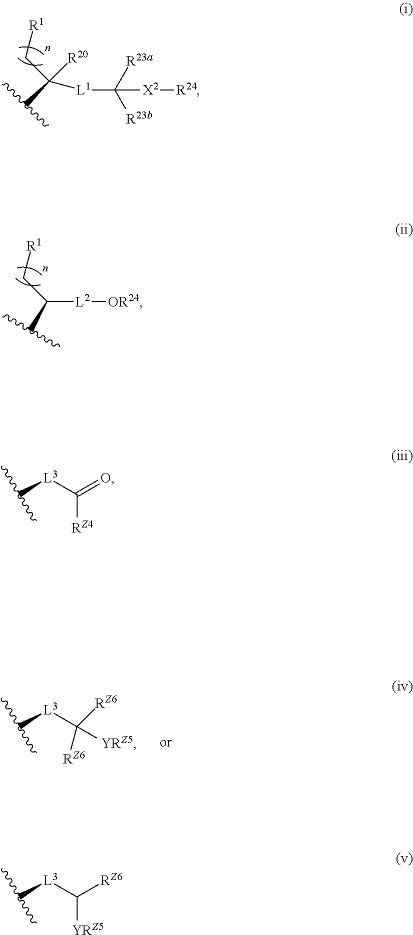

L¹ and L² are selected from a group consisting of a bond, a substituted or unsubstituted C₁-C₆ alkylene, a substituted or unsubstituted C₂-C₆ alkenylene, substituted or unsubstituted C₂-C₆ alkynylene, a substituted or unsubstituted hetero C₁-C₆ alkylene, a substituted or unsubstituted hetero C₂-C₆ alkenylene, and a substituted or unsubstituted hetero C₂-C₆ alkynylene;

L³ is a substituted or unsubstituted C₁-C₆ alkylene, a substituted or unsubstituted C₂-C₆ alkenylene, substituted or unsubstituted C₂-C₆ alkynylene, a substituted or unsubstituted hetero C₁-C₆ alkylene, a substituted or unsubstituted hetero C₂-C₆ alkenylene, or a substituted or unsubstituted hetero C₂-C₆ alkynylene;

each instance of X¹ and X² is independently —O—, —S—, —N(R^{X})—, wherein each instance of R^{X} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroalkyl, or an amino protecting group;

R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, halo, —N₃, —NO₂, —SCN, —CN, —OR^{A1}, —SR^{A1}, —N(R^{A1})₂, —N═NR^{A1}, —N═C(R^{A1})₂, —N(OR^{A1})(R^{A1}), —C(═O)R^{A1}, —C(═O)OR^{A1}, —C(═O)SR^{A1}, —C(═O)N(R^{A1})₂, —C(═O)N(OR^{A1})(R^{A1}), —OC(═O)R^{A1}, —OC(═O)OR^{A1}, —OC(═O)SR^{A1}, —OC(═O)N(R^{A1})₂, —NR^{A1}C(═O)R^{A1}, —NR^{A1}C(═O)OR^{A1}, —NR^{A1}C(═O)SR^{A1}, —NR^{A1}C(═O)N(R^{A1})₂, —SC(═O)R^{A2}, —SC(═O)OR^{A1}, —SC(═O)SR^{A1}, —SC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, —NR^{A1}SO₂R^{A2}, or —SO₂N(R^{A1})₂, wherein R^{A1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{A1} groups are joined to form an substituted or unsubstituted heterocyclic ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or an R^{A1} group and an R^{A2} group are joined to form an substituted or unsubstituted heterocyclic ring;

each instance of R², R^{4a}, R^{4b}, R^{7a}, R^{7b}, R^{11a}, and R^{11b} is independently hydrogen, —OH, halo, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —N₃, —NO₂, —SCN, —CN, —OR^{B1}, —SR^{B1}, —N(R^{B1})₂, —N═NR^{B1}, —N═C(R^{B1})₂, —N(OR^{B1})(R^{B1}), —C(═O)R^{B1}, —C(═O)OR^{B1}, —C(═O)SR^{B1}, —C(═O)N(R^{B1})₂, —C(═O)N(OR^{B1})(R^{B1}), —OC(═O)R^{B1}, —OC(═O)OR^{B1}, —OC(═O)SR^{B1}, —OC(═O)N(R^{B1})₂, —NR^{B1}C(═O)R^{B1}, —NR^{B1}C(═O)OR^{B1}, —NR^{B1}C(═O)SR^{B1}, —NR^{B1}C(═O)N(R^{B1})₂, —SC(═O)R^{B2}, —SC(═O)OR^{B1}, —SC(═O)SR^{B1}, —SC(═O)N(R^{B1})₂, —OS(═O)₂R^{B2}, —OS(═O)₂OR^{B1}, —S—S(═O)₂R^{B2}, —S—S(═O)₂OR^{B1}, —S(═O)R^{B2}, —SO₂R^{B2}, —NR^{B1}SO₂R^{B2}, or —SO₂N(R^{B1})₂, wherein R^{B1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{B1} groups are joined to form an substituted or unsubstituted heterocyclic ring; and R^{B2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or an R^{B1} group and an R^{B2} group are joined to form an substituted or unsubstituted heterocyclic ring; or optionally wherein each of R^{4a} and R^{4b}, and/or R^{7a} and R^{7b}, and/or R^{11a} and R^{11b} are joined to form an oxo (═O) group;

R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

R^{3b} is hydrogen, —C(═O)R^{C1}, —C(═O)OR^{C1}, —C(═O)SR^{C1}, —C(═O)N(R^{C1})₂, —S(═O)₂R^{C2}, —S(═O)₂OR^{C1}, —P(═O)₂R^{C2}, —P(═O)₂OR^{C1}, —P(═O)(OR^{C1})₂, —P(═O)(R^{C2})₂, or —P(═O)(R^{C2})(OR^{C1}), wherein R^{C1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{C1} groups are joined to form an substituted or unsubstituted heterocyclic ring; and R^{C2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

each of R^{6a} and R^{6b} is independently hydrogen, halo, substituted or unsubstituted alkyl, substituted or unsubstituted alkenyl, or substituted or unsubstituted alkynyl, and [symbol] represents a single or double bond, provided if a double bond is present in Ring B, then one of R^{6a} or R^{6b} is absent, and provided if a single bond is present in Ring B, then the hydrogen at C5 is in the alpha or beta position;

R¹⁴ is hydrogen or substituted or unsubstituted alkyl;

R¹⁷ is hydrogen, halo, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or —OR^{D1}, wherein R^{D1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an oxygen protecting group;

each instance of R¹⁸, R¹⁹, and R²⁰ is independently hydrogen or substituted or unsubstituted alkyl;

and each instance of R^{23a} and R^{23b} is independently hydrogen, halogen, or substituted or unsubstituted alkyl, or R^{23a} and R^{23b} are joined together to form substituted or unsubstituted C₃-C₆ cycloalkyl;

R²⁴ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{E1}, —C(═O)OR^{E1}, —C(═O)SR^{E1}, —C(═O)N(R^{E1})₂, —S(═O)₂R^{E2}, —S(═O)₂OR^{E1}, —P(═O)₂R^{E2}, —P(═O)₂OR^{E1}, —P(═O)(OR^{E1})₂, —P(═O)(R^{E2})₂, or —P(═O)(R^{E2})(OR^{E1}), wherein R^{E1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{E1} groups are joined to form an substituted or unsubstituted heterocyclic ring; and R^{E2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;

Y is —O—, —S—, or —NR^{Z5}—;

R^{Z4} is independently substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{Z5}, —SR^{Z5}, or —N(R^{Z5})₂;

each instance of R^{Z5} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, a sulfur protecting group when attached to a sulfur atom, a nitrogen protecting group when attached to a nitrogen atom, or two R^{Z5} groups are joined to form a substituted or unsubstituted heterocyclic ring; and

each instance of R^{Z6} is independently hydrogen or substituted or unsubstituted alkyl, or two R^{Z6} groups are joined to form a C₃₋₆ carbocyclic ring; and

the subscript n is 0, 1, 2, or 3.

In certain embodiments, when R^{3a} is H, n is 1, and R¹⁹ is Me; then R¹ is other than H, alkyl, alkenyl, or alkynyl. In certain embodiments, when R^{3a} is H, R^{3b} is —COMe, R¹⁹ is Me, and n is 0; then R¹ is OH. In certain embodiments, when R^{3a} is H, n is 0, and R²⁰ is alkyl; then R¹ is other than OH. In certain embodiments, when R¹⁹ is Me; then R¹ is other than H, alkyl, alkenyl, or alkynyl. In certain embodiments, R¹ is H; and R¹⁹ is other than Me. In certain embodiments, each R¹ and R^{3a} is H; and R¹⁹ is other than Me.

In certain embodiments, when R^{3a} is H, then R¹ is other than H, substituted or unsubstituted alkyl, substituted or unsubstituted alkenyl, or substituted or unsubstituted alkynyl. In certain embodiments, when R^{3a} is H, then R¹ is substituted or unsubstituted carbocyclyl, substituted or unsubstituted heterocyclyl, substituted or unsubstituted aryl, substituted or unsubstituted heteroaryl, halo, —N₃, —NO₂, —SCN, —CN, —OR^{A1}, —SR^{A1}, —N(R^{A1})₂, —N═NR^{A1}, —N═C(R^{A1})₂, —N(OR^{A1})(R^{A1}), —C(═O)R^{A1}, —C(═O)OR^{A1}, —C(═O)SR^{A1}, —C(═O)N(R^{A1})₂, —C(═O)N(OR^{A1})(R^{A1}), —OC(═O)R^{A1}, —OC(═O)OR^{A1}, —OC(═O)SR^{A1}, —OC(═O)N(R^{A1})₂, —NR^{A1}C(═O)R^{A1}, —NR^{A1}C(═O)SR^{A1}, —NR^{A1}C(═O)N(R^{A1})₂, —SC(═O)R^{A2}, —SC(═O)OR^{A1}, —SC(═O)SR^{A1}, —SC(═O)N(R^{A1})₂, —OS(═O)₂R^{A2}, —OS(═O)₂OR^{A1}, —S—(═O)₂R^{A2}, —S—S(═O)₂OR^{A1}, —S(═O)R^{A2}, —SO₂R^{A2}, —SO₂R^{A2}, —NR^{A1}SO₂R^{A2}, or —SO₂N(R^{A1})₂.

### Various Embodiments of R^{3a}

As generally defined above, R^{3a} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. It is generally understood that R^{3a} may be in the alpha (down) or beta (up) position. In certain embodiments, R^{3a} is alpha. In certain embodiments, R^{3a} is beta.

In certain embodiments, R^{3a} is hydrogen.

In certain embodiments, R^{3a} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{3a} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃). In certain embodiments, R^{3a} is substituted alkyl, e.g., R^{3a} is haloalkyl, alkoxyalkyl, or aminoalkyl. In certain embodiments, R^{3a} is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl. In certain embodiments, R^{3a} is Me, Et, n-Pr, n-Bu, or i-Bu. In certain embodiments, R^{3a} is methoxymethyl, ethoxymethyl, propoxymethyl, methoxyethyl, or ethoxyethyl. In certain embodiments, R^{3a} is trifluoromethoxymethyl. In certain embodiments, R^{3a} is fluoromethyl, chloromethyl, difluoromethyl, trifluoromethyl, difluoroethyl, trifluoroethyl, or 2,2,2-trifluoro-1,1-dimethyl-ethyl. In certain embodiments, R^{3a} is trifluoromethyl.

In certain embodiments, R^{3a} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl. In certain embodiments, R^{3a} is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R^{3a} is ethenyl, propenyl, or butenyl, unsubstituted or substituted with alkyl, halo, haloalkyl, alkoxyalkyl, or hydroxy. In certain embodiments, R^{3a} is ethenyl.

In certain embodiments, R^{3a} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl. Exemplary substituted or unsubstituted R^{3a} alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R^{3a} is selected from the group consisting of trifluoroethenyl, cyclopropylethynyl, cyclobutylethynyl, and propynyl, fluoropropynyl, and chloroethynyl. In certain embodiments, R^{3a} is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R^{3a} is ethynyl (C₂), propynyl (C₃), or butynyl (C₄) substituted with substituted phenyl. In certain embodiment, the phenyl substitutent is further substituted with one or more substituents selected from the group consisting of halo, alkyl, trifluoroalkyl, alkoxy, acyl, amino or amido. In certain embodiments, R^{3a} is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R^{3a} is ethynyl, propynyl, or butynyl, unsubstituted or substituted with alkyl, halo, haloalkyl, alkoxyalkyl, or hydroxyl. In certain embodiments, R^{3a} is ethynyl or propynyl, substituted with substituted or unsubstituted aryl. In certain embodiments, R^{3a} is ethynyl or propynyl, substituted with phenyl unsubstituted or substituted with halo, alkyl, alkoxy, haloalkyl, trihaloalkyl, or acyl. In certain embodiments, R^{3a} is ethynyl or propynyl, substituted with substituted or unsubstituted carbocyclyl. In certain embodiments, R^{3a} is ethynyl or propynyl, substituted with substituted or unsubstituted cyclopropyl, cyclobutyl, cyclopentyl, or cyclohexyl. In certain embodiments, R^{3a} is ethynyl or propynyl, substituted with substituted or unsubstituted heteroaryl. In certain embodiments, R^{3a} is ethynyl or propynyl, substituted with substituted or unsubstituted pyridinyl, or pyrimidinyl. In certain embodiments, R^{3a} is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R^{3a} is ethynyl or propynyl, substituted with substituted or unsubstituted heterocyclyl. In certain embodiments, R^{3a} is ethynyl or propynyl, substituted with substituted or unsubstituted pyrrolidinyl, piperidinyl, piperazinyl, or morpholinyl. In certain embodiments, R^{3a} is propynyl or butynyl, substituted with hydroxyl or alkoxy. In certain embodiments, R^{3a} is propynyl or butynyl, substituted with methoxy or ethoxy. In certain embodiments, R^{3a} is ethynyl or propynyl, substituted with Cl. In certain embodiments, R^{3a} is ethynyl or propynyl, substituted with trifluoromethyl.

In certain embodiments, R^{3a} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, R^{3a} is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, R^{3a} is substituted or unsubstituted aryl. In certain embodiments, R^{3a} is substituted or unsubstituted phenyl.

In certain embodiments, R^{3a} is substituted or unsubstituted heteroaryl, e.g., optionally substituted 5- to 6-membered heteroaryl.

Further embodiments of R^{3a}, as a substituted or unsubstituted alkyl, substituted or unsubstituted alkenyl, and substituted or unsubstituted alkynyl groups, are depicted below:

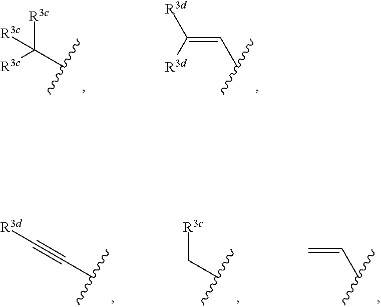

wherein each instance of R^{3c} is hydrogen, halo, or —OR^{F1}, wherein R^{F1} is substituted or unsubstituted alkyl; and each instance of R^{3d} is hydrogen, halo, or substituted or unsubstituted alkyl, substituted or unsubstituted carbocyclyl, or substituted or unsubstituted heterocyclyl.

In certain embodiments, at least one R^{3c} is hydrogen. In certain embodiments, at least two R^{3c} is hydrogen. In certain embodiments, each R^{3c} is hydrogen. In certain embodiments, at least one R^{3c} is halogen (e.g., fluoro, chloro, bromo, iodo). In certain embodiments, at least two R^{3c} are halogen (e.g., fluoro, chloro, bromo, iodo). In certain embodiments, each R^{3c} is halogen (e.g., fluoro, to provide the group —CF₃). In certain embodiments, at least one R^{3c} is —OR^{F1} (e.g., OMe or OEt). In certain embodiments, at least two R^{3c} is —OR^{F1} (e.g., OMe or OEt). In certain embodiments, at least one R^{3c} is hydrogen, F, —OMe, or —OEt. In certain embodiments, one of R^{3c} is F, —OMe, or OEt; and the rest are H.

In certain embodiments, at least one R^{3d} is hydrogen. In certain embodiments, each R^{2c} is hydrogen. In certain embodiments, at least one R^{3d} is halogen (e.g., fluoro, chloro, bromo, iodo). In certain embodiments, each R^{3d} is halogen (e.g., fluoro, chloro, bromo, iodo). In certain embodiments, each of R^{3d} is alkyl, e.g., each of R^{2c} is Me. In certain embodiments, one of R^{3d} is alkyl; and the other is hydrogen, e.g., one of R^{3d} is Me; and the other is hydrogen. In certain embodiments, one of R^{3d} is substituted or unsubstituted carbocyclyl, e.g., cyclopropyl or cyclobutyl, and the other is hydrogen. In certain embodiments, at least one R^{3d} is hydrogen, —F, —Br, —Cl, —I, —CH₃, —CF₃, cyclopropyl, or cyclobutyl. In certain embodiments, each instance of R^{3d} is H. In certain embodiments, each instance of R^{3d} is halogen (e.g., fluoro, chloro, bromo, iodo). In certain embodiments, each instance of R^{3d} is alkyl, e.g., —CH₃, —CF₃, —CH₂CH₂Cl. In certain embodiments, each instance of R^{3d} is substituted or unsubstituted carbocyclyl, e.g., cyclopropyl or cyclobutyl. In certain embodiments, R^{3d} is substituted or unsubstituted cyclopropyl. In certain embodiments, each instance of R^{3d} is hydrogen, —F, —Br, —Cl, —I, —CH₃, —CF₃, —CH₂CH₂Cl, cyclopropyl, or cyclobutyl. In certain embodiments, R^{3d} is Me or Cl. In certain embodiments, R^{3d} is substituted or unsubstituted heterocyclyl.

### Various Embodiments of —X¹—R^{3b}

As generally defined above, for group —X¹R^{3b}, X¹ is independently —O—, —S—, or —N(R^{X})—, wherein each instance of R^{X} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroalkyl, or an amino protecting group; and R^{3b} is hydrogen, —C(═O)R^{C1}, —C(═O)OR^{C1}, —C(═O)SR^{C1}, —C(═O)N(R^{C1})₂, —S(═O)₂R^{C1}, —S(═O)₂OR^{C1}, —P(═O)₂R^{C1}, —P(═O)₂OR^{C1}, —P(═O)(OR^{C1})₂, —P(═O)(R^{C1})₂, or —P(═O)(R^{C1})(OR^{C1}), wherein R^{C1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{C1} groups are joined to form an substituted or unsubstituted heterocyclic ring. It is generally understood that the group —X¹—R^{3b} may be in the alpha (down) or beta (up) position. In certain embodiments, the group —X¹—R^{3b} is alpha. In certain embodiments, the group —X¹—R^{3b} is beta.

In certain embodiments, X¹ is —O—. In certain embodiments, X¹ is —S—. In certain embodiments, X¹ is —N(R^{X})—. In certain embodiments, R^{X} is alkyl. In certain embodiments, R^{X} is Me, Et, or i-Pr. In certain embodiments, R^{X} is H, i.e., wherein X¹ is —NH—.

In certain embodiments, R^{3b} is hydrogen. For example, in certain embodiments, the group —X¹R^{3b} is —OH. In certain embodiments, the group —X¹R^{3b} is —SH. In certain embodiments, the group —X¹R^{3b} is —NH₂ or —NHR^{X}.

In certain embodiments, R^{3b} is —C(═O)R^{C1}, —C(═O)OR^{C1}, —C(═O)SR^{C1}, —C(═O)N(R^{C1})₂, —S(═O)₂R^{C1}, —S(═O)₂OR^{C1}, —P(═O)₂R^{C1}, —P(═O)₂OR^{C1}, —P(═O)(OR^{C1})₂, —P(═O)(R^{C1})₂, or —P(═O)(R^{C1})(OR^{C1}).

In certain embodiments, at least one instance of R^{C1} is hydrogen or a protecting group, i.e., an oxygen protecting group when attached to an oxygen atom, sulfur protecting group when attached to an sulfur atom, or a nitrogen protecting group when attached to a nitrogen atom. In certain embodiments, at least one instance of R^{C1} is hydrogen.

In certain embodiments, at least one instance of R^{C1} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{C1} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, at least one instance of R^{C1} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, at least one instance of R^{C1} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, at least one instance of R^{C1} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, at least one instance of R^{C1} is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, at least one instance of R^{C1} is substituted or unsubstituted aryl, e.g., substituted or unsubstituted phenyl.

In certain embodiments, at least one instance of R^{C1} is substituted or unsubstituted heteroaryl, e.g., optionally substituted 5- to 6-membered heteroaryl.

In certain embodiments, two R^{C1} groups are joined to form a substituted or unsubstituted heterocyclic ring, e.g., a substituted or unsubstituted piperidinyl, substituted or unsubstituted piperazinyl, or substituted or unsubstituted morpholinyl ring.

In certain embodiments, R^{3b} is —C(═O)R^{C1}, —C(═O)OR^{C1}, —C(═O)N(R^{C1})₂ or —C(═O)N(OR^{C1})(R^{C1}) wherein R^{C1} is as defined herein.

In certain embodiments, R^{3b} is —C(═O)R^{C1}, e.g., for example, wherein R^{C1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆). In certain embodiments, R^{3b} is —C(═O)CH₃. In certain embodiments, R^{3b} is —C(═O)(CH₂)ₘCO₂H, wherein m is an integer between 2 and 5, inclusive. In certain embodiments, m is 2. In certain embodiments, m is 3. In certain embodiments, m is 4. In certain embodiments, m is 5. In certain embodiments, R^{3b} is —C(═O)CH₂CH₂C(═O)OH.

In certain embodiments, R^{3b} is —C(═O)OR^{C1}, e.g., for example, wherein R^{C1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, R^{3b} is —C(═O)SR^{C1}, e.g., for example, wherein R^{C1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, R^{3b} is —C(═O)N(R^{C1})₂, e.g., —C(═O)NH₂ or —C(═O)NHR^{C1}, wherein R^{C1} is, for example, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or R′ is —C(═O)N(R^{C1})₂ wherein the two R^{C1} groups are joined to form a substituted or unsubstituted heterocyclic ring, e.g., substituted or unsubstituted piperidinyl, substituted or unsubstituted piperazinyl, or substituted or unsubstituted morpholinyl ring.

In certain embodiments, R^{3b} is —S(═O)₂R^{C1} or —S(═O)₂OR^{C1}, wherein R^{C1} is, for example, hydrogen, or substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In certain embodiments, R^{3b} is —S(═O)₂R^{C1}. In certain embodiments, R^{3b} is —S(═O)₂OR^{C1}, e.g., —SO₃H.

In certain embodiments, R^{3b} is —P(═O) R^{C1}, —P(═O)₂OR^{C1}, —P(═O)(OR^{C1})₂, —P(═O)(R^{C1})₂, or —P(═O)(R^{C1})(OR^{C1}), wherein each R^{C1} is, for example, independently hydrogen, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or substituted or unsubstituted phenyl. In certain embodiments, R^{3b} is —P(═O)₂R^{C1}. In certain embodiments, R¹ is —P(═O)₂OR^{C1}. In certain embodiments, R^{3b} is —P(═O)(OR^{C1})₂. In certain embodiments, R¹ is —P(═O)(R^{C1})₂. In certain embodiments, R^{3b} is —P(═O)(R^{C1})(OR^{C1}).

### Various Embodiments Wherein Z is a Group of Formula (i) or (ii)

In certain embodiments, Z is a group of formula (i):

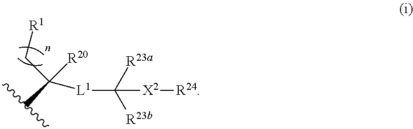

In other embodiments, Z is a group of formula (ii):

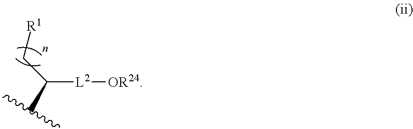

As generally defined above, L¹ and L² is a bond (i.e., in other words, is absent) or is a substituted or unsubstituted C₁-C₆ alkylene, a substituted or unsubstituted C₂-C₆ alkenylene, substituted or unsubstituted C₂-C₆ alkynylene, a substituted or unsubstituted hetero C₁-C₆ alkylene, a substituted or unsubstituted hetero C₂-C₆ alkenylene, or a substituted or unsubstituted hetero C₂-C₆ alkynylene.

In certain embodiments, L¹ or L² is a bond.

In certain embodiments, L¹ or L² is a substituted or unsubstituted C₁-C₆ alkylene. In certain embodiments, L¹ or L² is a substituted or unsubstituted C₁-C₄ alkylene. In certain embodiments, L¹ or L² is a substituted or unsubstituted C₁-C₃ alkylene. In certain embodiments, L¹ or L² is a substituted or unsubstituted C₁-C₂ alkylene. In certain embodiments, L¹ or L² is a substituted or unsubstituted C₁ alkylene. In certain embodiments, L¹ or L² is a substituted or unsubstituted C₂ alkylene. In certain embodiments, L¹ or L² is a substituted or unsubstituted C₃ alkylene. In certain embodiments, L¹ or L² is a substituted or unsubstituted C₄ alkylene. In certain embodiments, L¹ or L² is a substituted or unsubstituted C₅ alkylene. In certain embodiments, L¹ or L² is a substituted or unsubstituted C₆ alkylene. In certain embodiments, L¹ or L² is an alkylene group, as described above, substituted with one or more substituents selected from the group consisting of substituted or unsubstituted alkyl and halo. In certain embodiments, L¹ or L² is —CH₂—, —CHMe-, —CMe₂-, —CH₂—CH₂—, —CF₂—CH₂—, —CH₂—CMe₂-, —CH₂—CH₂—CH₂—, or —CH₂—CH₂—CMe₂-.

In certain embodiments, L¹ or L² is a substituted or unsubstituted C₂-C₆ alkenylene. In certain embodiments, L¹ or L² is a substituted or unsubstituted C₂-C₅ alkenylene. In certain embodiments, L¹ or L² is a substituted or unsubstituted C₂-C₄ alkenylene. In certain embodiments, L¹ or L² is a substituted or unsubstituted C₂-C₃ alkenylene. In certain embodiments, L¹ or L² is a substituted or unsubstituted C₂ alkenylene. In certain embodiments, L¹ or L² is a substituted or unsubstituted C₃ alkenylene. In certain embodiments, L¹ or L² is a substituted or unsubstituted C₄ alkenylene. In certain embodiments, L¹ or L² is a substituted or unsubstituted C₅ alkenylene. In certain embodiments, L¹ or L² is a substituted or unsubstituted C₆ alkenylene. In certain embodiments, L¹ or L² is an alkenylene group, as described above, substituted with one or more substituents selected from the group consisting of substituted or unsubstituted alkyl and halo.

In certain embodiments, L¹ or L² is a substituted or unsubstituted C₂-C₆ alkynylene. In certain embodiments, L¹ or L² is a substituted or unsubstituted C₂-C₅ alkynylene. In certain embodiments, L¹ or L² is a substituted or unsubstituted C₂-C₄ alkynylene. In certain embodiments, L¹ or L² is a substituted or unsubstituted C₂-C₃ alkynylene. In certain embodiments, L¹ or L² is a substituted or unsubstituted C₂ alkynylene. In certain embodiments, L¹ or L² is a substituted or unsubstituted C₃ alkynylene. In certain embodiments, L¹ or L² is a substituted or unsubstituted C₄ alkynylene. In certain embodiments, L′ is a substituted or unsubstituted C₅ alkynylene. In certain embodiments, L¹ or L² is a substituted or unsubstituted C₆ alkynylene. In certain embodiments, L¹ or L² is an alkynylene group, as described above, substituted with one or more substituents selected from the group consisting of substituted or unsubstituted alkyl and halo.

Furthermore, in certain embodiments, L¹ or L² is substituted or unsubstituted heteroC₁₋₆alkylene, e.g., substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. In certain embodiments, L¹ or L² is substituted or unsubstituted heteroC₂₋₆alkenylene, e.g., substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene. In certain embodiments, L¹ or L² is substituted or unsubstituted heteroC₂₋₆alkynylene, e.g., substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene. In any of the above instances, in certain embodiments, L¹ or L² is heteroalkylene, heteroalkenylene, or heteroalkynylene unsubstituted or substituted with halo (e.g., fluoro) or substituted or unsubstituted C₁₋₆ alkyl.

As generally defined above, R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, halo, —N₃, —NO₂, —SCN, —CN, —OR^{A1}, —SR^{A1}, —N(R^{A1})₂, —N═NR^{A1}, —N═C(R^{A1})₂, —N(OR^{A1})(R^{A1}), —C(═O)R^{A1}, —C(═O)OR^{A1}, —C(═O)SR^{A1}, —C(═O)N(R^{A1})₂, —C(═O)N(OR^{A1})(R^{A1}), —OC(═O)R^{A1}, —OC(═O)OR^{A1}, —OC(═O)SR^{A1}, —OC(═O)N(R^{A1})₂, —NR^{A1}C(═O)R^{A1}, —NR^{A1}C(═O)OR^{A1}, —NR^{A1}C(═O)SR^{A1}, —NR^{A1}C(═O)N(R^{A1})₂, —SC(═O)R^{A2}, —SC(═O)OR^{A1}, —SC(═O)SR^{A1}, —SC(═O)N(R^{A1})₂, —OS(═O)₂R^{A2}, —OS(═O)₂OR^{A1}, —S—S(═O)₂R^{A2}, —S—S(═O)₂OR^{A1}, —S(═O)R^{A2}, —SO₂R^{A2}, —NR^{A1}SO₂R^{A2}, or —SO₂N(R^{A1})₂, wherein R^{A1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{A1} groups are joined to form an substituted or unsubstituted heterocyclic ring; and R^{A2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or an R^{A1} group and an R^{A2} group are joined to form an substituted or unsubstituted heterocyclic ring.

In certain embodiments, R¹ is hydrogen.

In certain embodiments, R¹ is substituted or unsubstituted alkyl, substituted or unsubstituted alkenyl or substituted or unsubstituted alkynyl. In certain embodiments, R¹ is substituted or unsubstituted alkyl, e.g., Me, Et, or i-Pr. In certain embodiments, R¹ is substituted or unsubstituted alkenyl, e.g., substituted or unsubstituted ethenyl or substituted or unsubstituted propenyl. In certain embodiments, R¹ is substituted or unsubstituted alkynyl.

In certain embodiments, R¹ is selected from substituted or unsubstituted carbocyclyl or substituted or unsubstituted heterocyclyl.

In certain embodiments, R¹ is substituted or unsubstituted aryl, e.g., phenyl.

In certain embodiments, R¹ is substituted or unsubstituted heteroaryl, e.g., a substituted or unsubstituted heteroaryl selected from pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl, pyridinyl, pyrimidinyl, pyrazinyl, quinolinyl, isoquinolinyl, quinazonyl, quinoxilinyl, naphthyridinyl, indolyl, indazolyl, benzimidazloyl, pyrrolopyridinyl, pyrrolopyrimidinyl, pyridopyrimidinyl, or purinyl. In certain embodiments, the heteroaryl group is substituted with one or more groups selected from substituted or unsubstituted alkyl, haloalkyl, alkenyl, substituted or unsubstituted alkynyl, oxo, hydoxy, halo, alkoxy, —S-alkyl, substituted or unsubstituted aryl, substituted or unsubstituted heteroaryl, substituted or unsubstituted —SO-alkyl, substituted or unsubstituted —SO₂-alkyl, substituted or unsubstituted —SO-aryl, substituted or unsubstituted —SO₂-aryl, substituted or unsubstituted —SO-heteroaryl, substituted or unsubstituted —SO₂-heteroaryl, amino, cyano, and acyl. In certain embodiments, R¹ is imidazolyl, pyrazolyl, 1,2,3-triazolyl, 1,2,4-triazolyl, oxadiazolyl, thiadiazolyl, or tetrazolyl; each unsubstituted or substituted with one or two groups independently selected from oxo, Me, F, Cl, —CN, and —CF₃. In certain embodiments, R¹ is quinolinyl, isoquinolinyl or purinyl; each unsubstituted or substituted with one or two groups independently selected from oxo, Me, F, Cl, —CN, and —CF₃.

In certain embodiments, R¹ is —OR^{A1}. In certain embodiments, R¹ is —O-quinolinyl, isoquinolinyl, —O-purinyl, each unsubstituted or substituted with one or two groups independently selected from Me, F, Cl, —CN, and —CF₃. In certain embodiments, R¹ is —OH or —O—CO—CH₂—CH₂—CO₂H.

In certain embodiments, R¹ is —SR^{A1}. In certain embodiments, R¹ is S-quinolinyl, —S-isoquinolinyl, or —S-purinyl, each unsubstituted or substituted with one or two groups independently selected from Me, F, Cl, —CN, and —CF₃. In certain embodiments, R¹ is —SH.

In certain embodiments, R¹ is —OS(═O)₂R^{A2}. In certain embodiments, R¹ is —OS(═O)₂OR^{A1}; e.g., —O—SO₃H. In certain embodiments, R¹ is —S—S(═O)₂R^{A2}. In certain embodiments, R¹ is —S—S(═O)₂OR^{A1}; e.g., —S—SO₃H.

As generally defined above, R²⁰ is independently hydrogen or substituted or unsubstituted alkyl. In certain embodiments, R²⁰ is hydrogen. In certain embodiments, R²⁰ is substituted or unsubstituted alkyl (e.g., —CH₃).

As generally defined above each instance of R^{23a} and R^{23b} is independently hydrogen, halogen, or substituted or unsubstituted alkyl, or R^{23a} and R^{23b} are joined together to form substituted or unsubstituted C₃-C₆ cycloalkyl. In certain embodiments, each instance of R^{23a} and R^{23b} is hydrogen. In certain embodiments, one of R^{23a} and R^{23b} is halogen, e.g., fluoro, and the other of R^{23a} and R^{23b} is hydrogen, halogen, or substituted or unsubstituted alkyl. In certain embodiments, each instance of R^{23a} and R^{23b} is halogen, e.g., fluoro. In certain embodiments, each instance of R^{23a} and R^{23b} is independently substituted or unsubstituted alkyl. In certain embodiments, each of R^{23a} and R^{23b} is Me. In certain embodiments, one of R^{23a} and R^{23b} is H. In certain embodiments, one of R^{23a} and R^{23b} is H; and the other is substituted or unsubstituted alkyl. In certain embodiments, one of R^{23a} and R^{23b} is H; and the other is Me or Et. In certain embodiments, R^{23a} and R^{23b} are joined together to form substituted or unsubstituted C₃-C₆ cycloalkyl. In certain embodiments, R^{23a} and R^{23b} are joined together to form a substituted or unsubstituted cyclopropyl.

In certain embodiments, the group

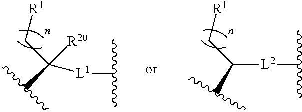

is of the formula:

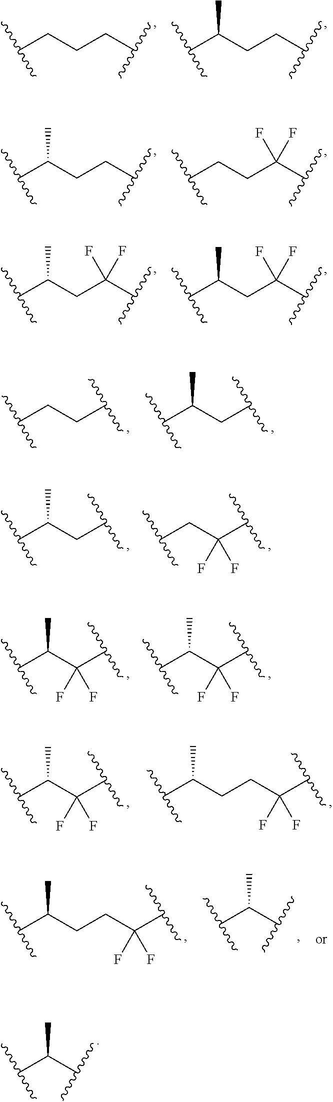

As generally defined above, X² is independently —O—, —S—, or —N(R^{X})—, wherein each instance of R^{X} is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroalkyl, or an amino protecting group.

In certain embodiments, X² is —O—. In certain embodiments, X² is —S—. In certain embodiments, X² is —N(R^{X})—. In certain embodiments, R^{X} is alkyl. In certain embodiments, R^{X} is Me, Et, or i-Pr. In certain embodiments, R^{X} is hydrogen.

In certain embodiments, X¹ is —O— and X² is —O—. In certain embodiments, X¹ is —O— and X² is —S—. In certain embodiments, X¹ is —O— and X² is —N(R^{X})—. In certain embodiments, X¹ is —S— and X² is —O—. In certain embodiments, X¹ is —S— and X² is —S—. In certain embodiments, X¹ is —S— and X² is —N(R^{X})—. In certain embodiments, X¹ is —N(R^{X})— and X² is —O—. In certain embodiments, X¹ is —N(R^{X})— and X² is —S—. In certain embodiments, X¹ is —N(R^{X})— and X² is —N(R^{X})—.

As generally defined above, R²⁴ is H, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{E1}, —C(═O)OR^{E1}, —C(═O)SR^{E1}, —C(═O)N(R^{E1})₂, —S(═O)₂R^{E2}, —S(═O)₂OR^{E1}, —P(═O)₂R^{E2}, —P(═O)₂OR^{E1}, —P(═O)(OR^{E1})₂, —P(═O)(R^{E2})₂, or —P(═O)(R^{E2})(OR^{E1}).

In certain embodiments, R²⁴ is hydrogen.

In certain embodiments, R²⁴ is substituted or unsubstituted alkyl. In certain embodiments, R²⁴ is alkyl unsubstituted or substituted with one or more substituents selected from the group consisting of halo or and hydroxyl. In certain embodiments, R²⁴ is substituted or unsubstituted alkenyl. In certain embodiments, R²⁴ is substituted or unsubstituted alkynyl. In certain embodiments, R²⁴ is substituted or unsubstituted carbocyclyl. In certain embodiments, R²⁴ is substituted or unsubstituted heterocyclyl. In certain embodiments, R²⁴ is substituted or unsubstituted aryl. In certain embodiments, R²⁴ is substituted or unsubstituted heteroaryl.

In certain embodiments, R²⁴ is —C(═O)R^{E1}, e.g., R²⁴ is —C(═O)(CH₂)ₚCO₂H, wherein p is an integer between 2 and 5, inclusive. In certain embodiments, p is 2. In certain embodiments, p is 3. In certain embodiments, p is 4. In certain embodiments, p is 5. In certain embodiments, R²⁴ is —C(═O)OR^{E1}. In certain embodiments, R²⁴ is —C(═O)SR^{E1}. In certain embodiments, R²⁴ is —C(═O)N(R^{E1})₂. In certain embodiments, R²⁴ is —S(═O)₂R^{E2}. In certain embodiments, R²⁴ is —S(═O)₂OR^{E1}; e.g., —SO₃H. In certain embodiments, R²⁴ is —P(═O)₂R^{E2}. In certain embodiments, R²⁴ is —P(═O)₂OR^{E1}. In certain embodiments, R²⁴ is —P(═O)(R^{E1})₂. In certain embodiments, R²⁴ is —P(═O)(R^{E2})₂. In certain embodiments, R²⁴ is —P(═O)(R^{E2})(OR^{E1}).

As generally defined above, the subscript n is 0, 1, 2, or 3. In certain embodiments, n is 0. In certain embodiments, n is 1. In certain embodiments, n is 2. In certain embodiments, n is 3.

### Various Embodiments Wherein Z is a Group of Formula (iii), (iv), or (v)

In certain embodiments, Z is a group of formula (iii), (iv), or (v):

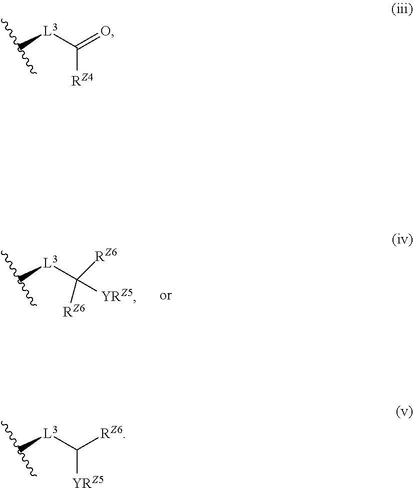

In certain embodiments, L³ is substituted or unsubstituted C₁₋₆alkylene, e.g., substituted or unsubstituted C₁₋₂alkylene, substituted or unsubstituted C₂₋₃alkylene, substituted or unsubstituted C₃₋₄alkylene, substituted or unsubstituted C₄₋₅alkylene, or substituted or unsubstituted C₅₋₆alkylene. In certain embodiments, L³ is substituted or unsubstituted C₂₋₆alkenylene, e.g., substituted or unsubstituted C₂₋₃alkenylene, substituted or unsubstituted C₃₋₄alkenylene, substituted or unsubstituted C₄₋₅alkenylene, or substituted or unsubstituted C₅₋₆alkenylene. In certain embodiments, L³ is substituted or unsubstituted C₂₋₆alkynylene, e.g., substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene. In any of the above instances, in certain embodiments, L³ is alkylene, alkenylene, or alkynylene unsubstituted or substituted with halo (e.g., fluoro), substituted or unsubstituted C₁₋₆ alkyl, and/or —OR^{Z5}.

Furthermore, in certain embodiments, L³ is substituted or unsubstituted heteroC₁₋₆alkylene, e.g., substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅alkylene, or substituted or unsubstituted heteroC₅₋₆alkylene. In certain embodiments, L³ is substituted or unsubstituted heteroC₂₋₆alkenylene, e.g., substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene. In certain embodiments, L³ is substituted or unsubstituted heteroC₂₋₆alkynylene, e.g., substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene. In any of the above instances, in certain embodiments, L³ is heteroalkylene, heteroalkenylene, or heteroalkynylene unsubstituted or substituted with halo (e.g., fluoro), substituted or unsubstituted C₁₋₆ alkyl, and/or —OR^{Z5}.

In any of the above or below instances, in certain embodiments, at least one R^{Z5} is hydrogen.

In any of the above or below instances, in certain embodiments, at least one instance of R^{Z5} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z5} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In any of the above or below instances, in certain embodiments, at least one instance of R^{Z5} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In any of the above or below instances, in certain embodiments, at least one instance of R^{Z5} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In any of the above or below instances, in certain embodiments, at least one instance of R^{Z5} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In any of the above or below instances, in certain embodiments, at least one instance of R^{Z5} is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In any of the above or below instances, in certain embodiments, at least one instance of R^{Z5} is substituted or unsubstituted aryl, e.g., substituted or unsubstituted phenyl.

In any of the above or below instances, in certain embodiments, at least one instance of R^{Z5} is substituted or unsubstituted heteroaryl, e.g., optionally substituted 5-6 membered heteroaryl.

In any of the above or below instances, in certain embodiments, R^{Z5} is a protecting group, e.g., an oxygen protecting group when attached to an oxygen atom, a sulfur protecting group when attached to a sulfur atom, a nitrogen protecting group when attached to a nitrogen atom.

In certain embodiments, wherein two R^{Z5} are attached to a nitrogen atom, the two R^{Z5} groups are joined to form a substituted or unsubstituted heterocyclic ring, e.g., a substituted or unsubstituted piperidinyl, substituted or unsubstituted piperazinyl, or substituted or unsubstituted morpholinyl ring.

Furthermore, in any of the above or below instances, in certain embodiments, each instance of R^{Z6} is independently hydrogen, substituted or unsubstituted alkyl, or two R^{Z6} groups are joined to form a C₃₋₆ carbocyclic ring.

In certain embodiments, at least one instance of R^{Z6} is hydrogen.

In certain embodiments, at least one instance of R^{Z6} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z4} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, two R^{Z6} groups are joined to form a C₃₋₆ carbocyclic ring, e.g., for example, a substituted or unsubstituted cyclopropyl, substituted or unsubstituted cyclobutyl, substituted or unsubstituted cyclopentyl, or substituted or unsubstituted cyclohexyl ring.

In certain embodiments, R^{Z4} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z4} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃).

In certain embodiments, R^{Z4} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R^{Z4} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R^{Z4} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, R^{Z4} is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, R^{Z4} is substituted or unsubstituted aryl, e.g., substituted or unsubstituted phenyl.

In certain embodiments, R^{Z4} is substituted or unsubstituted heteroaryl, e.g., optionally substituted 5- to 6-membered heteroaryl.

In certain embodiments, R^{Z4} is —OR^{Z5}, wherein R^{Z5} is as defined herein, e.g., for example, R^{Z5} is hydrogen, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, R^{Z4} is —SR^{Z5}, wherein R^{Z5} is as defined herein, e.g., for example, R^{Z5} is hydrogen, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆).

In certain embodiments, R^{Z4} is —N(R^{Z5})₂, e.g., R^{Z4} is —NH₂, or —NHR^{Z5}, wherein R^{Z5} is as defined herein, e.g., for example, R^{Z5} is hydrogen, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), or n-hexyl (C₆), or R^{Z4} is —N(R^{Z5})₂ wherein the two R^{Z5} groups are joined to form a substituted or unsubstituted heterocyclic ring, e.g., a substituted or unsubstituted piperidinyl, substituted or unsubstituted piperazinyl, or substituted or unsubstituted morpholinyl ring.

Specific L³ alkylene groups are contemplated herein. For example, in certain embodiments, L³ is an alkylene group of the formula:

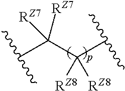

wherein p is 1, 2, or 3; and each instance of R^{Z7} and R^{Z8} is, independently, hydrogen, halo, substituted or unsubstituted C₁₋₆ alkyl, or —OR^{Z5}. In certain embodiments, p is 1. In certain embodiments, p is 2. In certain embodiments, p is 3.

Specific L³ alkenylene groups are also contemplated herein. For example, in certain embodiments, L³ is an alkenylene group of the formula:

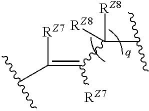

wherein q is 0, 1, or 2; and each instance of R^{Z7} and R^{Z8} is, independently, hydrogen, halo, substituted or unsubstituted C₁₋₆ alkyl, or —OR^{Z5}. In certain embodiments, q is 0. In certain embodiments, q is 1. In certain embodiments, q is 2.

Specific L³ heteroalkylene groups are also contemplated herein, e.g., for example, in certain embodiments, L³ is a heteroalkylene group of the formula:

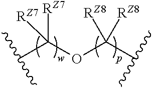

wherein w is 0 or 1 and p is 1, 2, or 3, or w is 1 and p is 0, 1, 2, or 3; and each instance of R^{Z7} and R^{Z8} is independently hydrogen, halo, substituted or unsubstituted C₁₋₆ alkyl, or —OR^{Z5}.

In certain embodiments, p is 0. In certain embodiments, p is 1. In certain embodiments, p is 2. In certain embodiments, p is 3. In certain embodiments, w is 0. In certain embodiments, w is 1. In certain embodiments, w is 0, and p is 1. In certain embodiments, w is 0, and p is 2. In certain embodiments, w is 0, and p is 3. In certain embodiments, w is 1, and p is 1. In certain embodiments, w is 1, and p is 2. In certain embodiments, w is 1, and p is 3.

For example, in certain embodiments wherein w is 0, provided is an L³ heteroalkylene group of the formula:

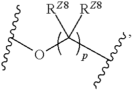

wherein p and R^{Z8} are as defined herein.

In certain embodiments wherein w is 1, provided is an L³ heteroalkylene group of the formula:

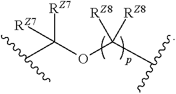

wherein p, R²⁷, and R^{Z8} are as defined herein.

In certain embodiments, at least one instance of R^{Z7} is hydrogen. In any of the above instances, in certain embodiments, at least one instance of R^{Z7} is halo, e.g., fluoro. In any of the above instances, in certain embodiments, at least one instance of R^{Z7} is substituted or unsubstituted C₁₋₆ alkyl, e.g., substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z7} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃). In any of the above instances, in certain embodiments, at least one instance of R^{Z7} is —CH₃, —CF₃, —CH₂CH₃ (Et), or —CH(CH₃)₂ (iPr). In any of the above instances, in certain embodiments, at least one instance of R^{Z7} is —OR^{Z5}, e.g., —OH.

In certain embodiments, at least one instance of R^{Z8} is hydrogen. In any of the above instances, in certain embodiments, at least one instance of R^{Z8} is halo, e.g., fluoro. In any of the above instances, in certain embodiments, at least one instance of R^{Z8} is substituted or unsubstituted C₁₋₆ alkyl, e.g., substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{Z8} C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃ and —CH₂OCH₂CH₃). In any of the above instances, in certain embodiments, at least one instance of R^{Z8} is —CH₃, —CF₃, —CH₂CH₃ (Et), or —CH(CH₃)₂ (iPr). In any of the above instances, in certain embodiments, at least one instance of R^{Z8} is —OR^{Z5}, e.g., —OH.

Exemplary L³ alkylene groups include, but are not limited to:

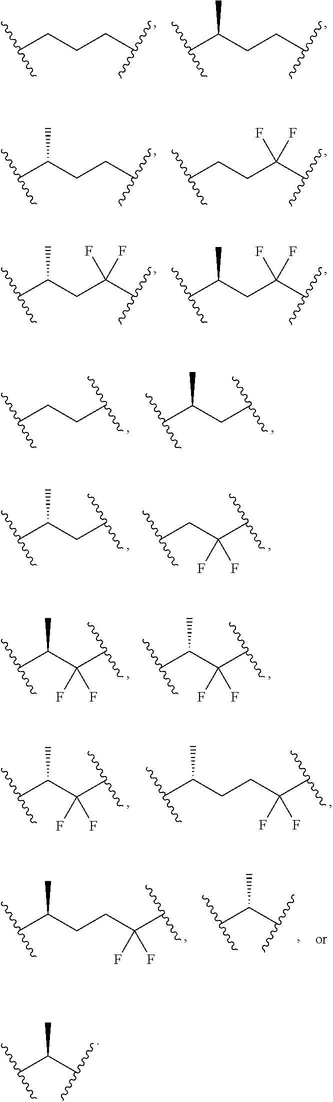

Exemplary L³ alkenylene groups include, but are not limited to:

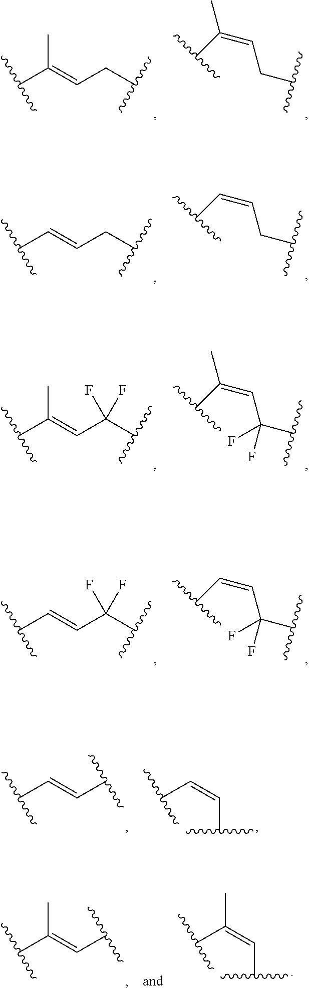

Exemplary L³ heteroalkylene groups include, but are not limited to:

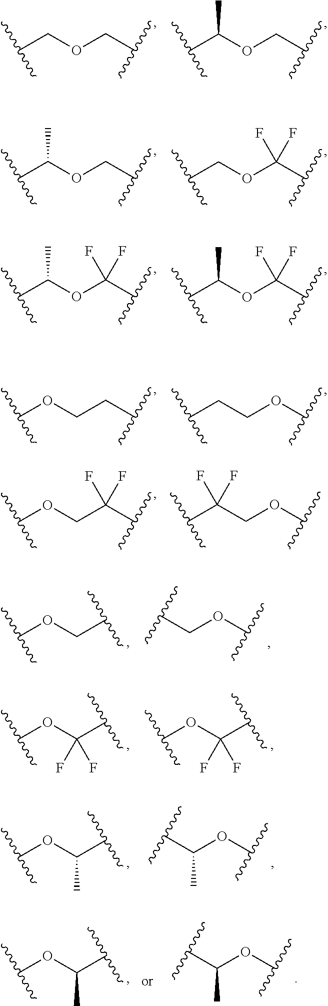

In certain embodiments, the group

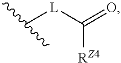

wherein L³ is an alkylene or heteroalkylene group, is of the formula:

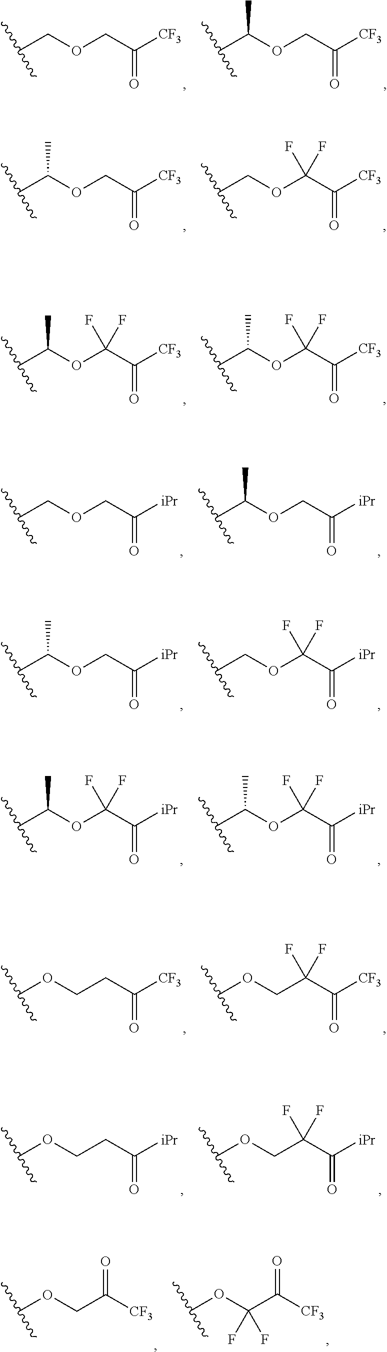

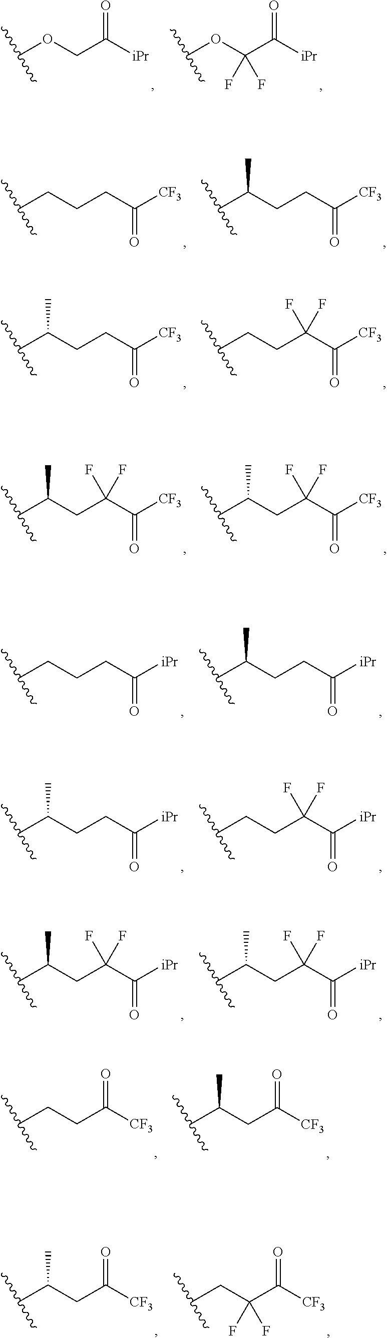

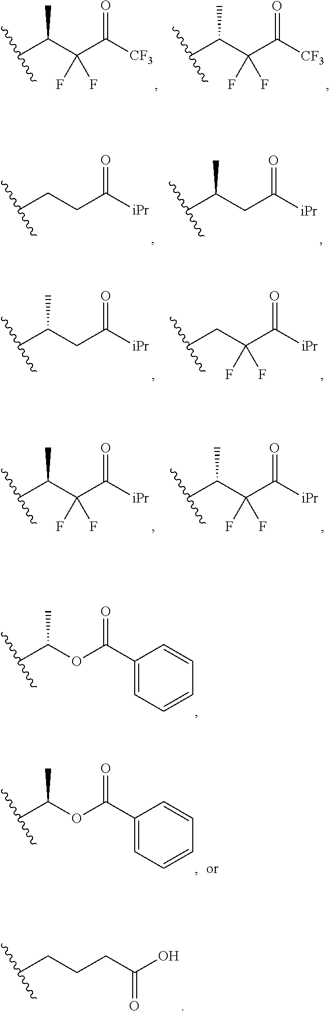

In certain embodiments, the group

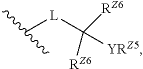

wherein Y is —O— and L³ is an alkylene or heteroalkylene group, is of the formula:

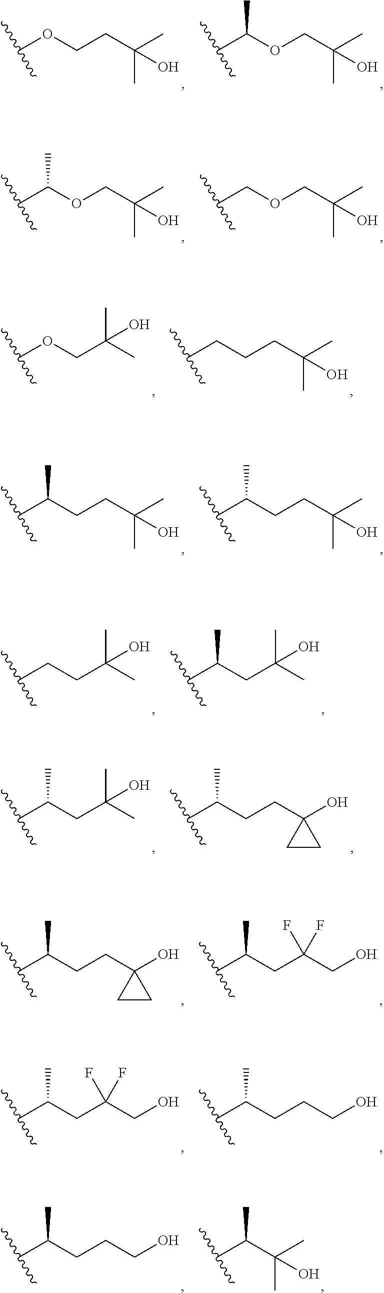

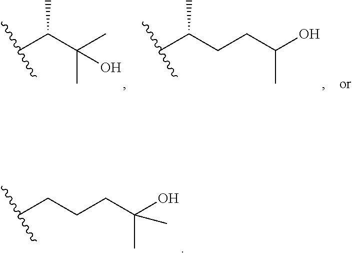

In certain embodiments, the group

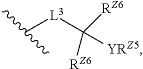

wherein Y is —NH— and L³ is an alkylene or heteroalkylene group, is of the formula

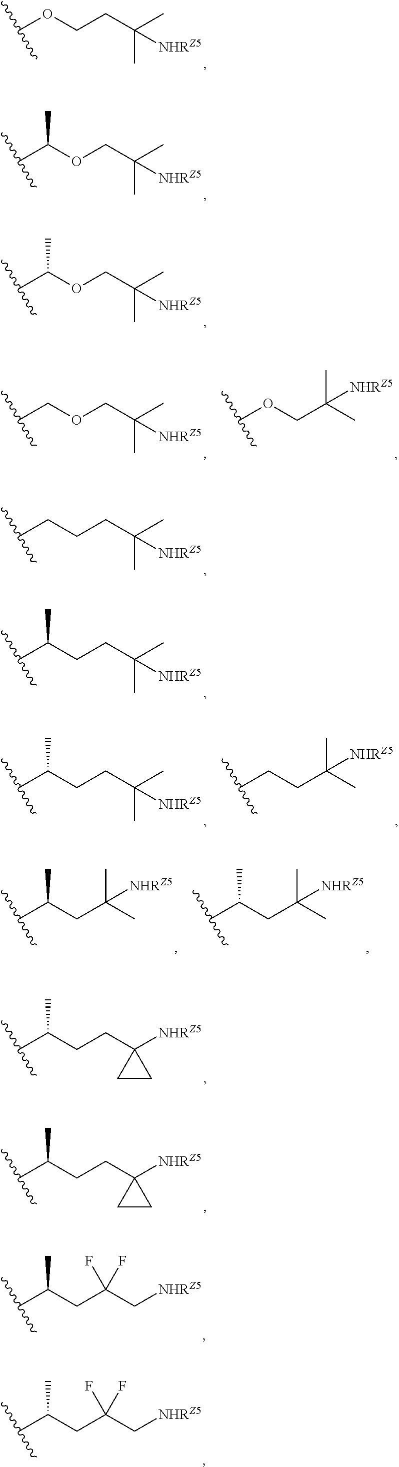

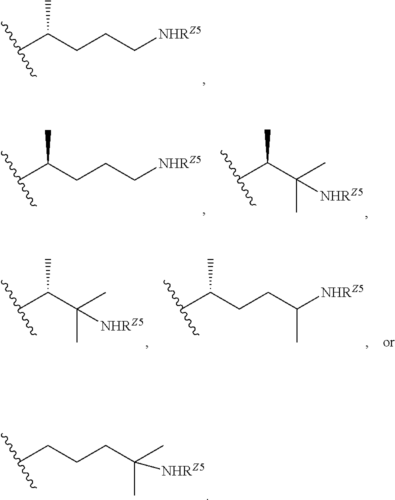

In certain embodiments, the group

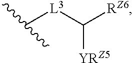

wherein Y is —O— and L³ is an alkylene or heteroalkylene group, is of the formula:

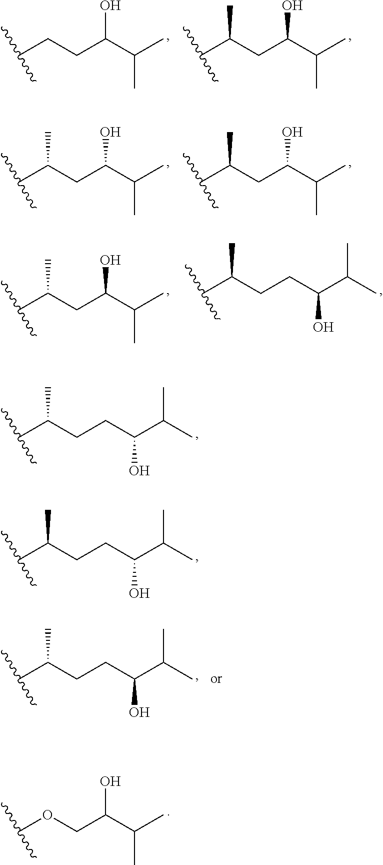

In certain embodiments, the group

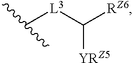

wherein Y is —NH— and L³ is an alkylene or heteroalkylene group, is of the formula:

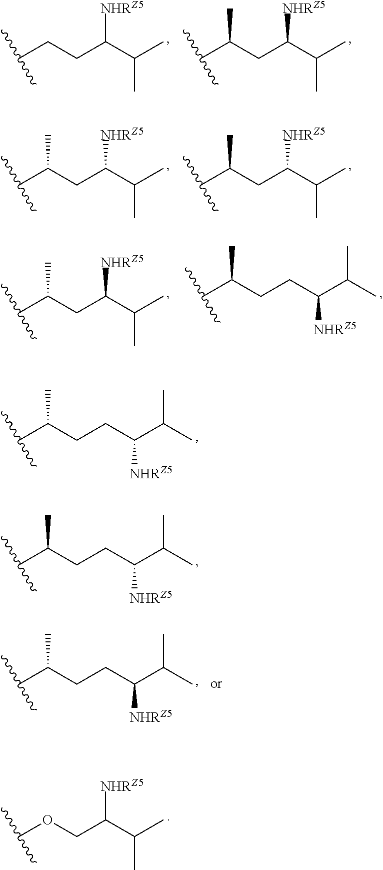

### Various Embodiments of R², R^{11a}, and R^{11b}

As generally defined above, each instance of R², R^{11a}, and R^{11b} is independently H, —OH, halo, substituted or unsubstituted alkyl, substituted or unsubstituted alkenyl, or substituted or unsubstituted alkynyl, substituted or unsubstituted carbocyclyl, substituted or unsubstituted heterocyclyl, substituted or unsubstituted aryl, substituted or unsubstituted heteroaryl, —N₃, —NO₂, —SCN, —CN, —OR^{B1}, —SR^{B1}, —N(R^{B1})₂, —N═NR^{B1}, —N═C(R^{B1})₂, —N(OR^{B1})(R^{B1}), —C(═O)R^{B1}, —C(═O)OR^{B1}, —C(═O)SR^{B1}, —C(═O)N(R^{b1})₂, —C(═O)N(OR^{B1})(R^{B1}), —OC(═O)R^{B1}, —OC(═O)OR^{B1}, —OC(═O)SR^{B1}, —OC(═O)N(R^{B1})₂, —NR^{B1}C(═O)R^{B1}, —NR^{B1}C(═O)OR^{B1}, —NR^{B1}C(═O)SR^{B1}, —NR^{B1}C(═O)N(R^{B1})₂, —SC(═O)R^{B2}, —SC(═O)OR^{B1}, —SC(═O)SR^{B1}, —SC(═O)N(R^{B1})₂, —OS(═O)₂R^{B2}, —OS(═O)₂OR^{B1}, —S—S(═O)₂R^{B2}, —S—S(═O)₂OR^{B1}, —S(═O)R^{B2}, —SO₂R^{B2}, —NR^{B1}SO₂R^{B2}, or —SO₂N(R^{B1})₂, and/or R^{11a} and R^{11b} are joined to form an oxo (═O) group

In certain embodiments, R² is H. In certain embodiments, R² is substituted or unsubstituted alkyl. In certain embodiments, R² is substituted or unsubstituted alkenyl. In certain embodiments, R² is substituted or unsubstituted alkynyl. In certain embodiments, R² is —OR^{B1}. In certain embodiments, R² is —SR^{B1}. In certain embodiments, R² is —N(R^{B1})₂. In certain embodiments, R² is H, halo, substituted or unsubstituted alkyl, substituted or unsubstituted alkenyl, substituted or unsubstituted alkynyl, —OR^{B1}, —SR^{B1}, or —N(R^{B1})₂. In certain embodiments, R² is F, Cl, Me, Et, n-Pr, methoxy, ethoxy, propoxy, butoxy, ethynyl, hydroxybutynyl, methoxypropynyl, chloroethynyl, or cyclopropynyl. In certain embodiments, R² is CF₃, amino, or dimethylamino. In certain embodiments, R² is a non-hydrogen group in the alpha position. In certain embodiments, R² is a non-hydrogen group in the beta position.

In certain embodiments, each instance of R^{11a} and R^{11b} is hydrogen. In certain embodiments, one of R^{11a} and R^{11b} is hydrogen. In certain embodiments, one of R^{11a} and R^{11b} is hydrogen; and the other is —OR^{B1}, —SR^{B1}, or —N(R^{B1})₂. In certain embodiments, one of R^{11a} and R^{11b} is H; and the other is —OH, —OMe, amino, or dialkylamino. In certain embodiments, R^{11b} is a non-hydrogen group, and R^{11a} is hydrogen. In certain embodiments, R^{11a} is a non-hydrogen group, and R^{11b} is hydrogen.

In certain embodiments, R^{11a} and R^{11b} together form an oxo group.

### Various Embodiments of R^{4a}, R^{4b}, R⁶, R^{7a}, R^{7b}, R¹⁴, R¹⁷, R¹⁸, and R¹⁹

As generally defined above, each instance of R^{4a}, R^{4b}, R^{7a}, and R^{7b} is independently hydrogen, —OH, halo, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —N₃, —NO₂, —SCN, —CN, —OR^{B1}, —SR^{B1}, —N(R^{B1})₂, —N═NR^{B1}, —N═C(R^{B1})₂, —N(OR^{B1})(R^{B1}), —C(═O)R^{B1}, —C(═O)OR^{B1}, —C(═O)SR^{B1}, —C(═O)N(R^{b1})₂, —C(═O)N(OR^{B1})(R^{B1}), —OC(═O)R^{B1}, —OC(═O)OR^{B1}, —OC(═O)SR^{B1}, —OC(═O)N(R^{B1})₂, —NR^{B1}C(═O)R^{B1}, —NR^{B1}C(═O)OR^{B1}, —NR^{B1}C(═O)SR^{B1}, —NR^{B1}C(═O)N(R^{B1})₂, —SC(═O)R^{B2}, —SC(═O)OR^{B1}, —SC(═O)SR^{B1}, —SC(═O)N(R^{B1})₂, —OS(═O)₂R^{B2}, —OS(═O)₂OR^{B1}, —S—S(═O)₂R^{B2}, —S—S(═O)₂OR^{B1}, —S(═O)R^{B2}, —SO₂R^{B2}, —NR^{B1}SO₂R^{B2}, or —SO₂N(R^{B1})₂, wherein R^{B1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, an oxygen protecting group when attached to an oxygen atom, a sulfur protecting group when attached to a sulfur atom, a nitrogen protecting group when attached to a nitrogen atom, or two R^{B1} groups are joined to form an substituted or unsubstituted heterocyclic ring; and R^{B2} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, or an R^{B1} group and an R^{B2} group are joined to form an substituted or unsubstituted heterocyclic ring; or optionally wherein each of R^{4a} and R^{4b}, and/or R^{7a} and R^{7b} are joined to form an oxo (═O) group.

In certain embodiments, each instance of R^{4a} and R^{4b} is hydrogen. In certain embodiments, one of R^{4a} and R^{4b} is hydrogen. In certain embodiments, one of R^{4a} and R^{4b} is hydrogen; and the other is substituted or unsubstituted alkyl, substituted or unsubstituted alkenyl, or substituted or unsubstituted alkynyl. In certain embodiments, one of R^{4a} and R^{4b} is hydrogen; and the other is Me, Et, ethenyl, ethynyl, propenyl, or propynyl. In certain embodiments, each of R^{4a} and R^{4b} is independently substituted or unsubstituted alkyl. In certain embodiments, each of R^{4a} and R^{4b} is Me.

In certain embodiments, each instance of R^{7a} and R^{7b} is hydrogen.

As generally defined above, each of R^{7a} and R^{6b} is independently hydrogen, halo, substituted or unsubstituted alkyl, substituted or unsubstituted alkenyl, or substituted or unsubstituted alkynyl, and [symbol] represents a single or double bond, provided if a double bond is present in Ring B, then one of R^{6a} or R^{6b} is absent, and provided if a single bond is present in Ring B, then the hydrogen at C5 is in the alpha or beta position.

In certain embodiments, wherein [symbol] represents a single bond, each instance of R^{6a} and R^{6b} is hydrogen. In certain embodiments, each instance of R^{6a} and R^{6b} is halo, e.g., fluoro.

In certain embodiments, wherein [symbol] represents a single bond, R^{6a} is hydrogen, and R^{6b} is halo, substituted or unsubstituted alkyl, substituted or unsubstituted alkenyl, or substituted or unsubstituted alkynyl. In certain embodiments, R^{6a} is hydrogen, and R^{6b} is halo (e.g., fluoro). In certain embodiments, R^{6a} is hydrogen, and R^{6b} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl, e.g., methyl, ethyl, propyl, or isopropyl. In certain embodiments, R^{6a} is hydrogen, and R^{6b} is substituted or unsubstituted alkenyl. In certain embodiments, R^{6a} is hydrogen, and R^{6b} is substituted or unsubstituted alkynyl.

In certain embodiments, wherein [symbol] represents a single bond, R^{6b} is hydrogen, and R^{6a} is halo, substituted or unsubstituted alkyl, substituted or unsubstituted alkenyl, or substituted or unsubstituted alkynyl. In certain embodiments, R^{6b} is hydrogen, and R^{6a} is halo (e.g., fluoro). In certain embodiments, R^{6b} is hydrogen, and R^{6a} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl, e.g., methyl, ethyl, propyl, or isopropyl. In certain embodiments, R^{6b} is hydrogen, and R^{6a} is substituted or unsubstituted alkenyl. In certain embodiments, R^{6b} is hydrogen, and R^{6a} is substituted or unsubstituted alkynyl.

In certain embodiments, wherein [symbol] represents a double bond, R^{6a} is hydrogen. In certain embodiments, wherein [symbol] represents a double bond, R^{6a} is halo, e.g., fluoro. In certain embodiments, wherein [symbol] represents a double bond, R^{6a} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl, e.g., methyl, ethyl, propyl, or isopropyl. In certain embodiments, wherein [symbol] represents a double bond, R^{6a} is substituted or unsubstituted alkenyl. In certain embodiments, wherein [symbol] represents a double bond, R^{6a} is substituted or unsubstituted alkynyl.

As generally defined above, R¹⁷ is hydrogen, halo, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or —OR^{D1}. In certain embodiments, R¹⁷ is hydrogen. In certain embodiments, R¹⁷ is halo. In certain embodiments, R¹⁷ is substituted or unsubstituted alkyl. In certain embodiments, R¹⁷ is substituted or unsubstituted alkenyl. In certain embodiments, R¹⁷ is substituted or unsubstituted alkynyl. In certain embodiments, R¹⁷ is substituted or unsubstituted carbocyclyl. In certain embodiments, R¹⁷ is substituted or unsubstituted heterocyclyl. In certain embodiments, R¹⁷ is substituted or unsubstituted aryl. In certain embodiments, R¹⁷ is substituted or unsubstituted heteroaryl. In certain embodiments, R¹⁷ is —OR^{D1} (e.g., —OH).

As generally defined above, R¹⁴ is H or substituted or unsubstituted alkyl. In certain embodiments, R¹⁴ is H. In certain embodiments, R¹⁴ is substituted or unsubstituted alkyl (e.g., —CH₃).

As generally defined above, R¹⁸ is independently hydrogen or substituted or unsubstituted alkyl. In certain embodiments, R¹⁸ is hydrogen. In certain embodiments, R¹⁸ is substituted or unsubstituted alkyl (e.g., —CH₃).

As generally defined above, R¹⁹ is independently hydrogen or substituted or unsubstituted alkyl. In certain embodiments, R¹⁹ is hydrogen. In certain embodiments, R¹⁹ is substituted or unsubstituted alkyl (e.g., —CH₃).

In certain embodiments, R¹⁴ is hydrogen, R¹⁸ is —CH₃ and R¹⁹ is —CH₃.

In certain embodiments, R¹⁴ is hydrogen, R¹⁸ is —CH₃ and R¹⁹ is hydrogen.

### Additional Embodiments of Formula (I)

Various combinations of the above embodiments are further contemplated herein. For example, in certain embodiments, the compound of Formula (I) is of Formula (I-w):

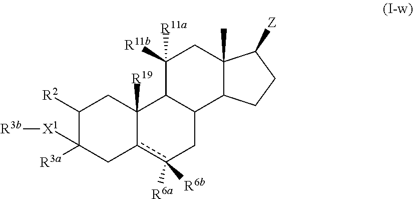

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, the group —X¹R^{3b} at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl.

In certain embodiments, the compound of Formula (I) is of Formula (I-x):

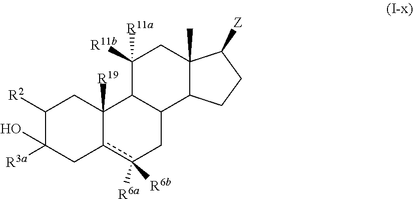

or a pharmaceutically acceptable salt, solvate, prodrug, stereoisomer, tautomer, isotopic variant, or N-oxide thereof, or a combination thereof. In certain embodiments, the group —OH at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl.

In certain embodiments, the compound of Formula (I) is of Formula (I-y):

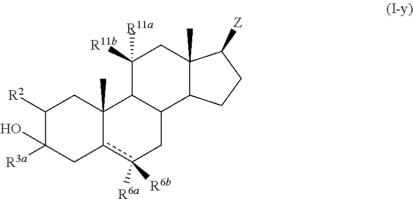

or a pharmaceutically acceptable salt, solvate, prodrug, stereoisomer, tautomer, isotopic variant, or N-oxide thereof, or a combination thereof. In certain embodiments, the group —OH at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro.

In certain embodiments, the compound of Formula (I) is of Formula (I-z):

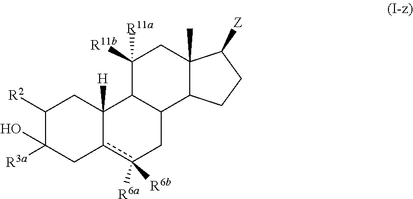

or a pharmaceutically acceptable salt, solvate, prodrug, stereoisomer, tautomer, isotopic variant, or N-oxide thereof, or a combination thereof. In certain embodiments, the group —OH at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro.

In certain embodiments, the compound of Formula (I) is of Formula (I-a1), (I-a2), or (I-a3):

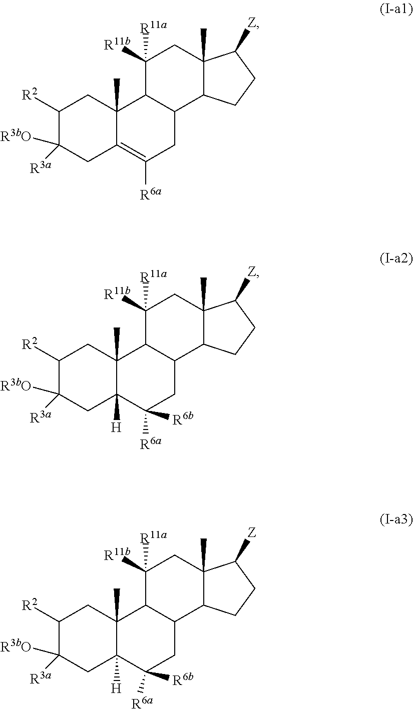

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, the group —OR^{3b} at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro.

In certain embodiments, the compound of Formula (I) is of Formula (I-b1), (I-b2), or (I-b3):

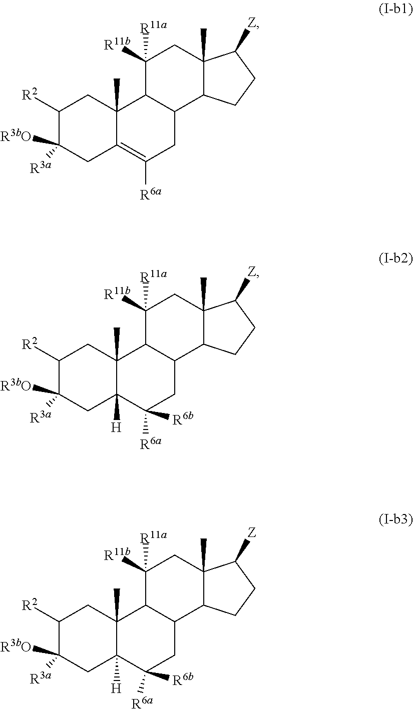

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro.

In certain embodiments, the compound of Formula (I) is of Formula (I-c1), (I-c2), or (I-c3):

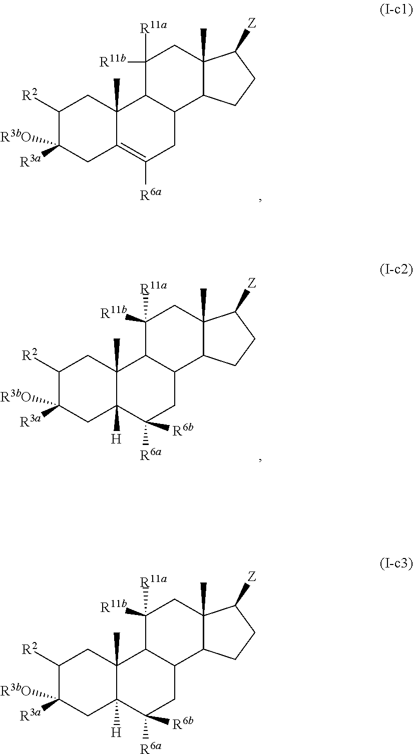

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro.

In certain embodiments, the compound is of Formula (I-d):

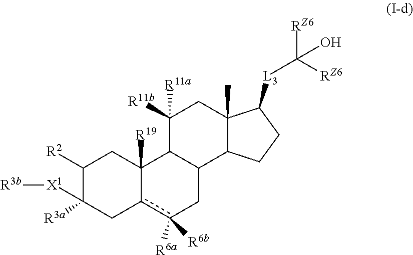

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, the group —X¹R^{3b} at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl. In certain embodiments, each R^{Z6} is independently hydrogen or methyl.

In certain embodiments, the compound is of Formula (I-e):

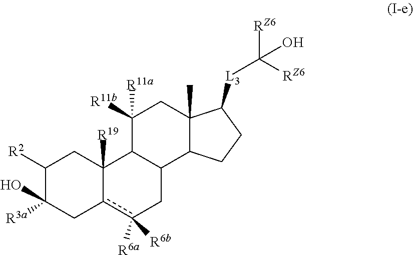

or a pharmaceutically acceptable salt, solvate, prodrug, stereoisomer, tautomer, isotopic variant, or N-oxide thereof, or a combination thereof. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl. In certain embodiments, each R^{Z6} is independently hydrogen or methyl.

In certain embodiments, the compound is of Formula (I-f):

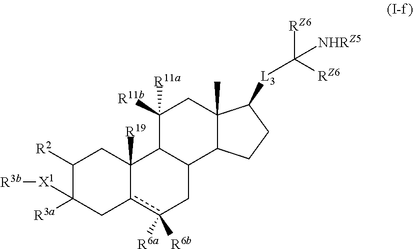

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, the group —X¹R^{3b} at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl. In certain embodiments, each R^{Z6} is independently hydrogen or methyl. In certain embodiments, R^{Z5} is hydrogen or methyl.

In certain embodiments, the compound is of Formula (I-g):

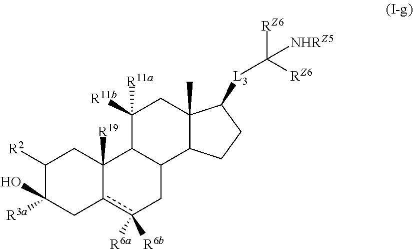

or a pharmaceutically acceptable salt, solvate, prodrug, stereoisomer, tautomer, isotopic variant, or N-oxide thereof, or a combination thereof. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl. In certain embodiments, each R^{Z6} is independently hydrogen or methyl. In certain embodiments, R^{Z5} is hydrogen or methyl.

In certain embodiments, the compound is of Formula (I-h):

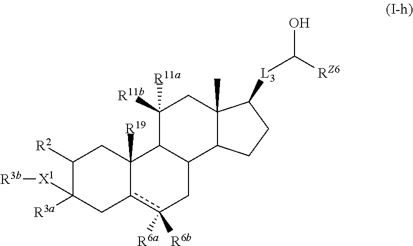

or a pharmaceutically acceptable salt, solvate, prodrug, stereoisomer, tautomer, isotopic variant, or N-oxide thereof, or a combination thereof. In certain embodiments, R^{3b} is hydrogen. In certain embodiments, the group —X¹R^{3b} at the C3 position is beta. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl. In certain embodiments, R^{Z6} is isopropyl.

In certain embodiments, the compound is of Formula (I-i):

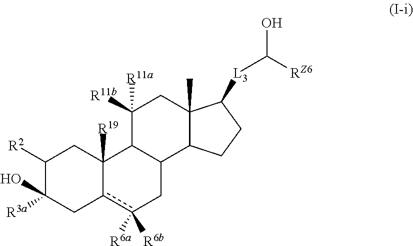

or a pharmaceutically acceptable salt, solvate, prodrug, stereoisomer, tautomer, isotopic variant, or N-oxide thereof, or a combination thereof. In certain embodiments, R^{3a} is hydrogen or substituted or unsubstituted alkyl. In certain embodiments, R² is hydrogen or —OR^{B1}. In certain embodiments, R^{11a} is hydrogen and R^{11b} is hydrogen or —OR^{B1}. In certain embodiments, [symbol] represents a single bond, R⁵ is alpha (down) and R^{6a} is hydrogen. In certain embodiments, [symbol] represents a double bond. In certain embodiments, R^{6a} and R^{6b} are both hydrogen. In certain embodiments, R^{6a} is halo, e.g., fluoro, or alkyl. In certain embodiments, R^{6b} is halo, e.g., fluoro, or alkyl, and R^{6a} is hydrogen. In certain embodiments, R^{6a} and R^{6b} are both halo, e.g., fluoro. In certain embodiments, R¹⁹ is methyl. In certain embodiments, R^{z6} is isopropyl.

Additional embodiments of Formula (I) include compounds of the following formula:

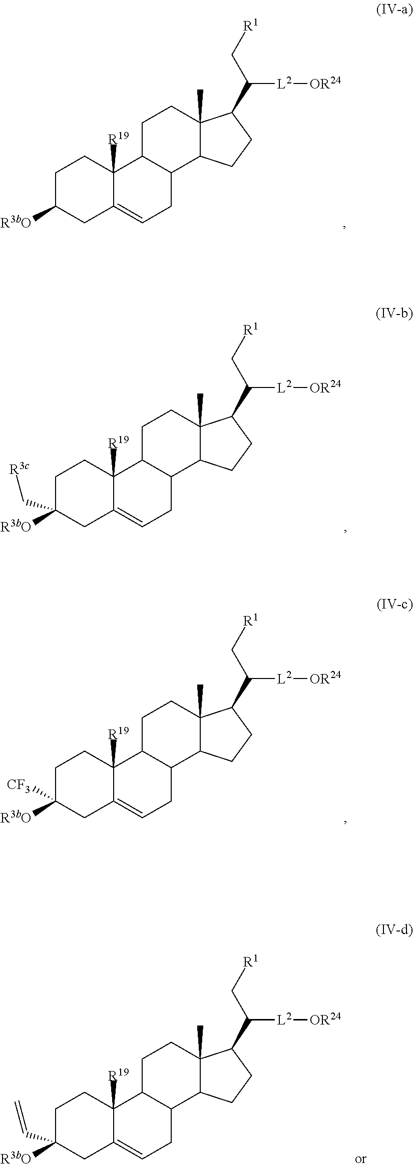

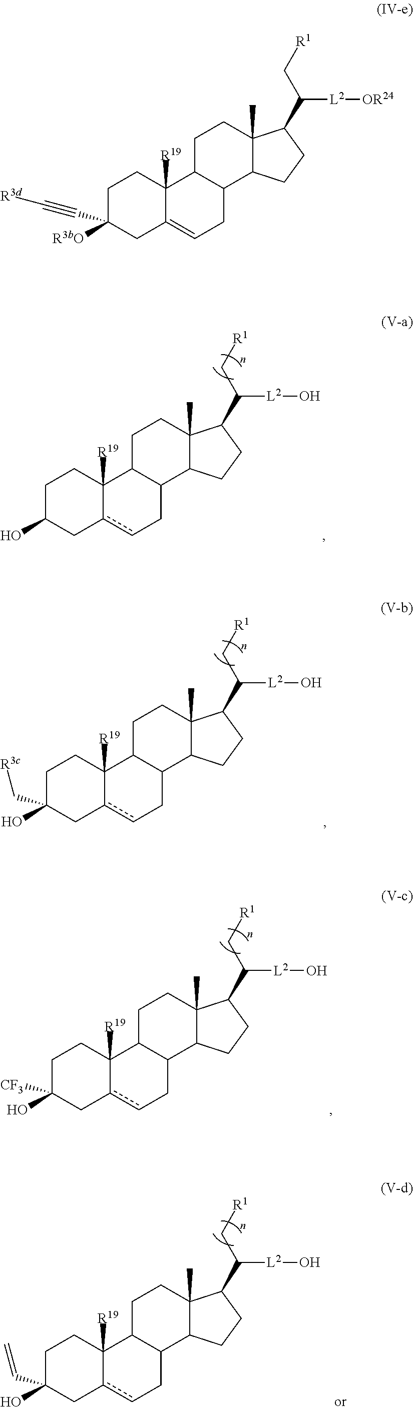

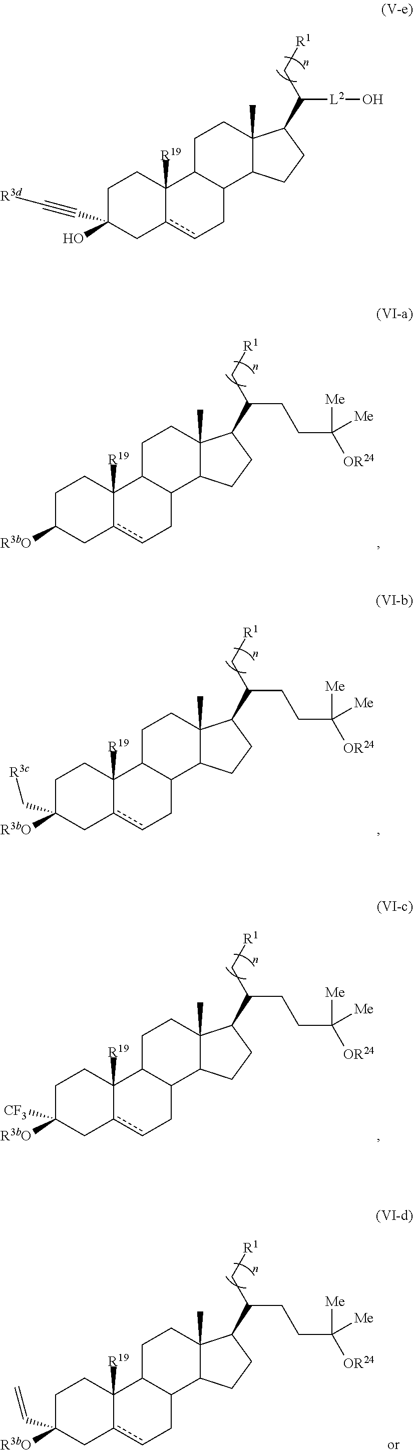

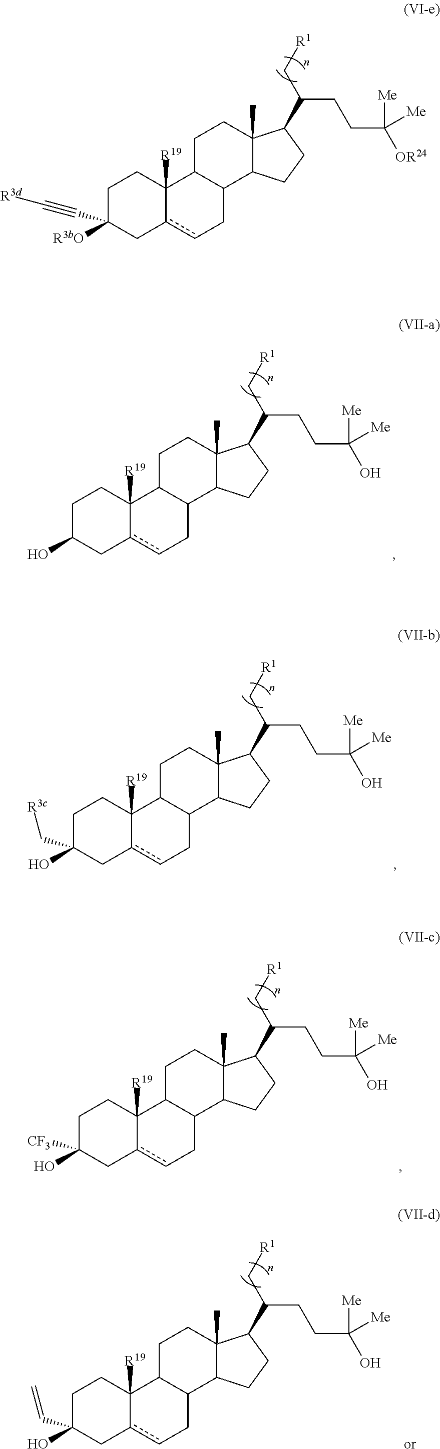

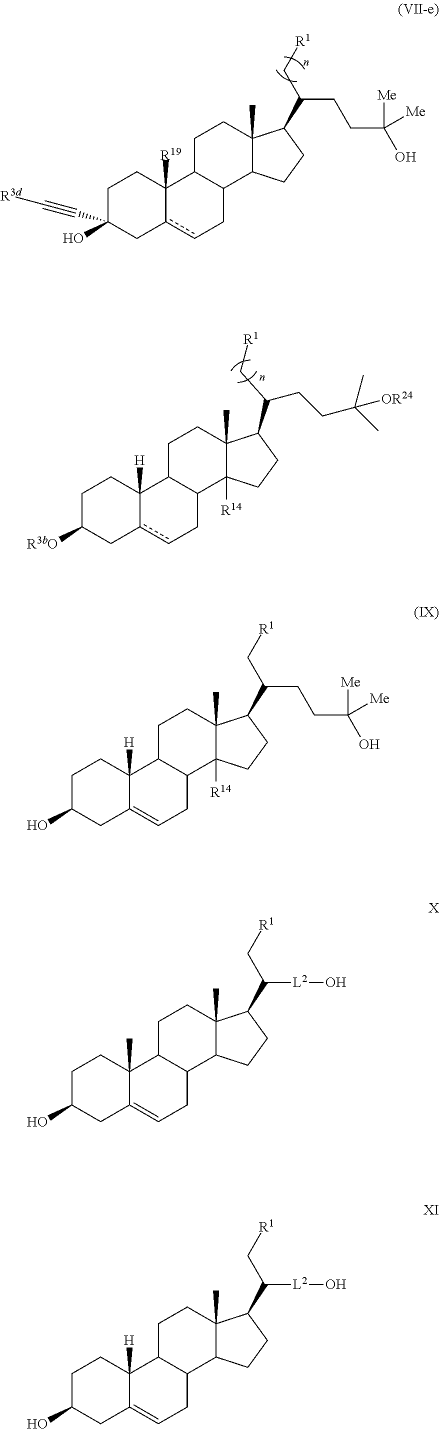

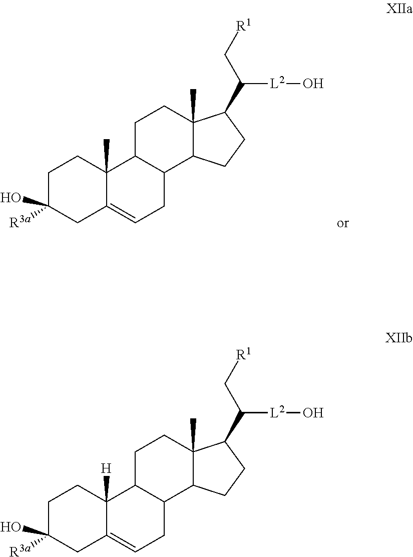

In certain embodiments the compound is any one of the following compounds:

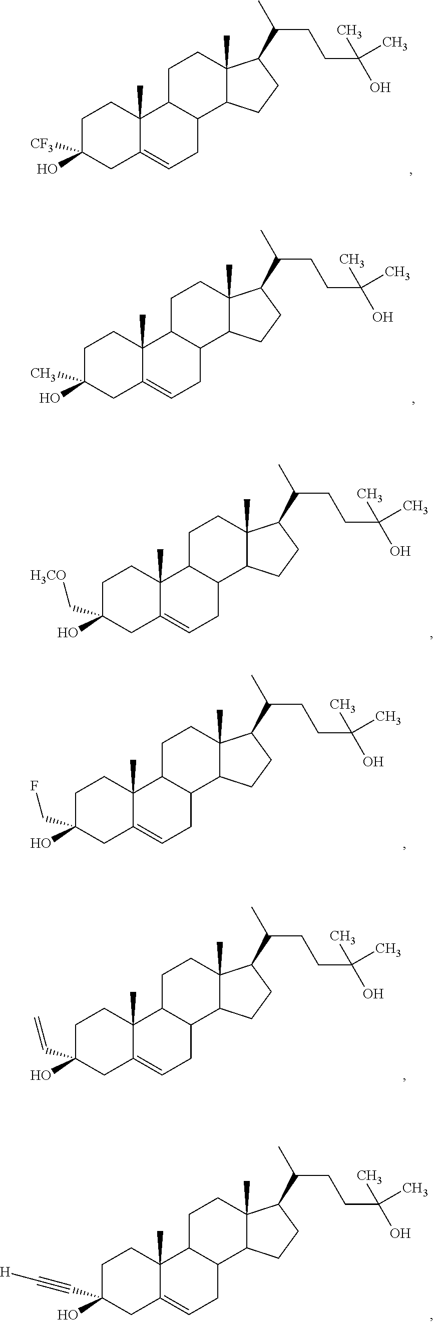

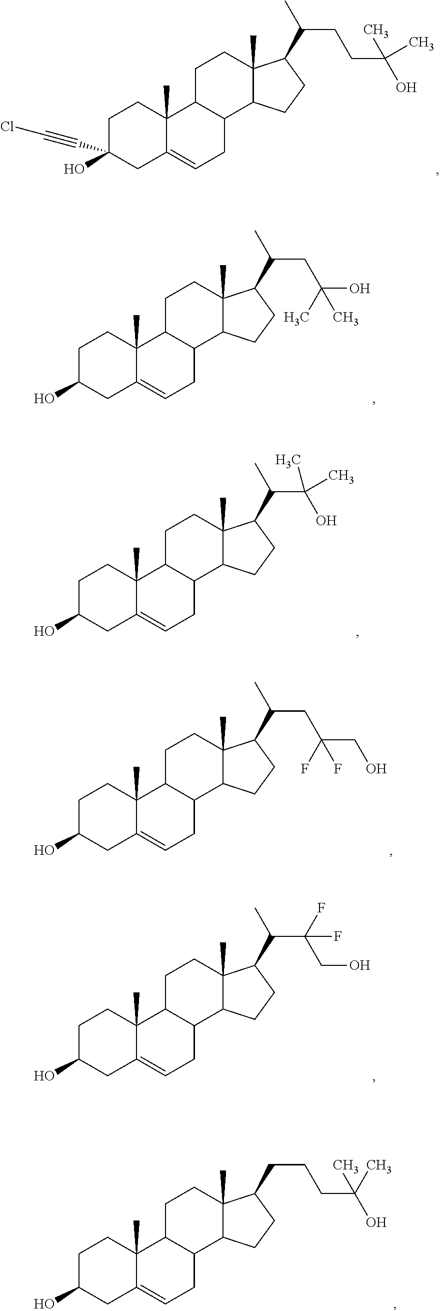 [SMILES: CC(CCC(C)(C)O)[C@H]1CCC2C3CC=C4C[C@](O)(C#CCl)CC[C@]4(C)C3CC[C@@]21C ; CC(CC(C)(C)O)[C@H]1CCC2C3CC=C4C[C@@H](O)CC[C@]4(C)C3CC[C@@]21C ; CC([C@H]1CCC2C3CC=C4C[C@@H](O)CC[C@]4(C)C3CC[C@@]21C)C(C)(C)O ; CC(CC(F)(F)CO)[C@H]1CCC2C3CC=C4C[C@@H](O)CC[C@]4(C)C3CC[C@@]21C ; CC([C@H]1CCC2C3CC=C4C[C@@H](O)CC[C@]4(C)C3CC[C@@]21C)C(F)(F)CO ; CC(C)(O)CCC[C@H]1CCC2C3CC=C4C[C@@H](O)CC[C@]4(C)C3CC[C@@]21C]

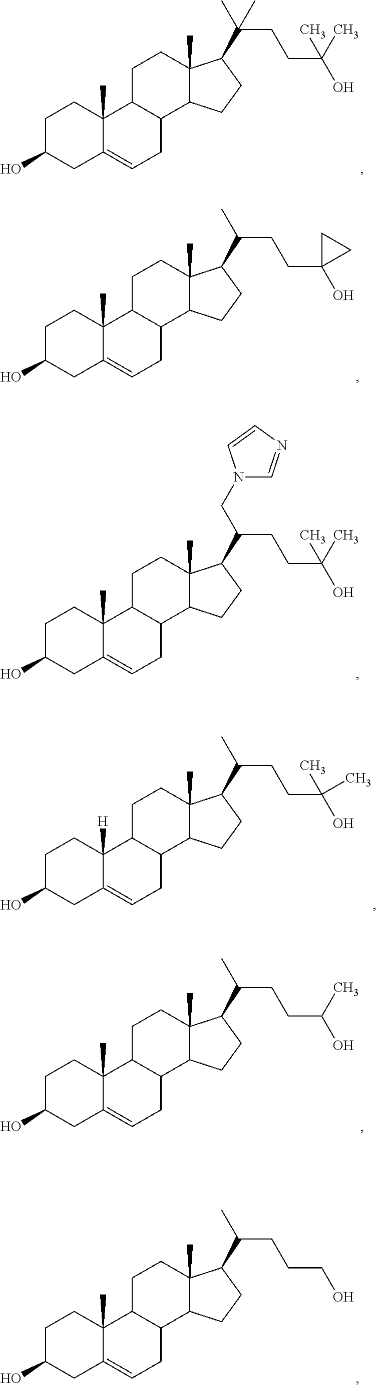 [SMILES: CC(C)(O)CCC(C)(C)[C@H]1CCC2C3CC=C4C[C@@H](O)CC[C@]4(C)C3CC[C@@]21C ; CC(CCC1(O)CC1)[C@H]1CCC2C3CC=C4C[C@@H](O)CC[C@]4(C)C3CC[C@@]21C ; CC(C)(O)CCC(Cn1ccnc1)[C@H]1CCC2C3CC=C4C[C@@H](O)CC[C@]4(C)C3CC[C@@]21C ; CC(CCC(C)(C)O)[C@H]1CCC2C3CC=C4C[C@@H](O)CC[C@@H]4C3CC[C@@]21C ; CC(O)CCC(C)[C@H]1CCC2C3CC=C4C[C@@H](O)CC[C@]4(C)C3CC[C@@]21C ; CC(CCCO)[C@H]1CCC2C3CC=C4C[C@@H](O)CC[C@]4(C)C3CC[C@@]21C]

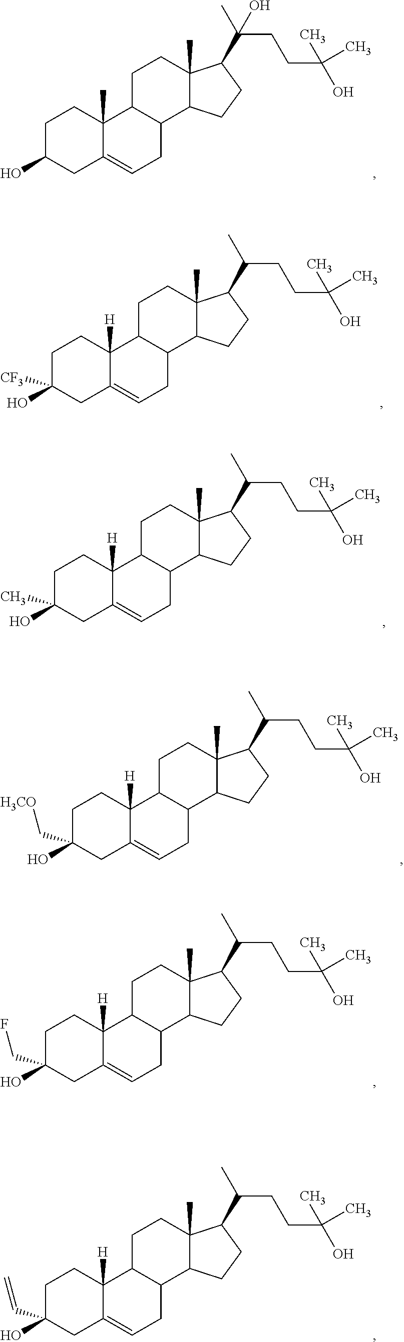

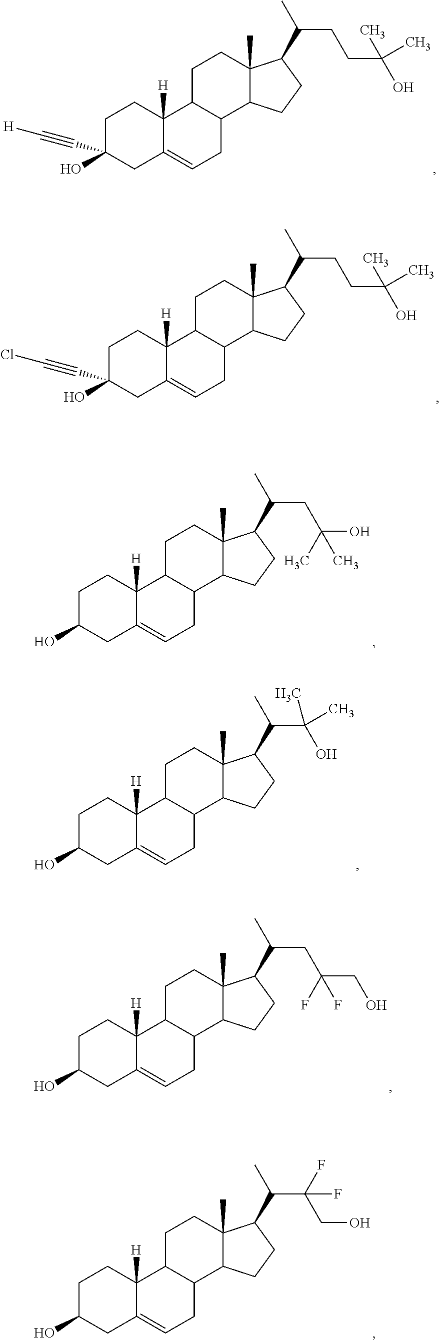 [SMILES: C#C[C@]1(O)CC[C@H]2C(=CCC3C2CC[C@@]2(C)C3CC[C@@H]2C(C)CCC(C)(C)O)C1 ; CC(CCC(C)(C)O)[C@H]1CCC2C3CC=C4C[C@](O)(C#CCl)CC[C@@H]4C3CC[C@@]21C ; CC(CC(C)(C)O)[C@H]1CCC2C3CC=C4C[C@@H](O)CC[C@@H]4C3CC[C@@]21C ; CC([C@H]1CCC2C3CC=C4C[C@@H](O)CC[C@@H]4C3CC[C@@]21C)C(C)(C)O ; CC(CC(F)(F)CO)[C@H]1CCC2C3CC=C4C[C@@H](O)CC[C@@H]4C3CC[C@@]21C ; CC([C@H]1CCC2C3CC=C4C[C@@H](O)CC[C@@H]4C3CC[C@@]21C)C(F)(F)CO]

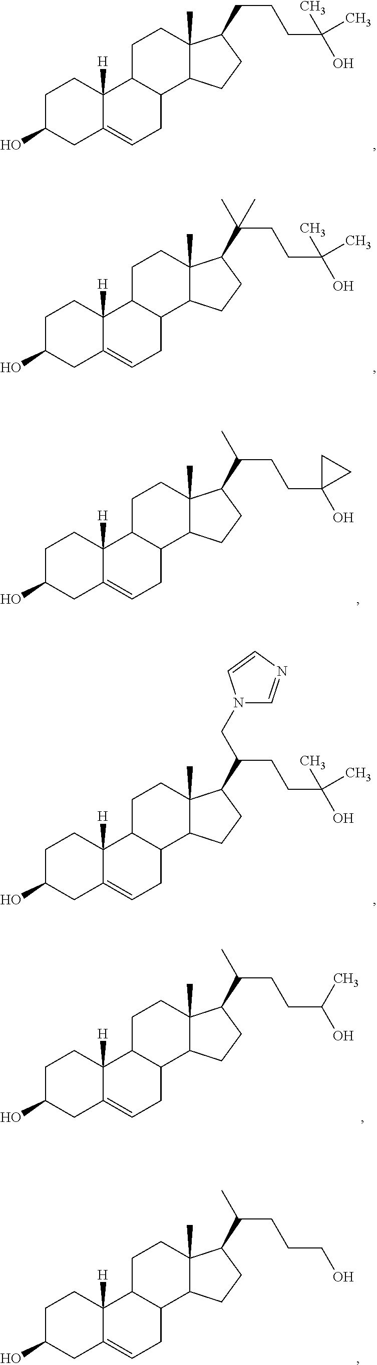 [SMILES: CC(C)(O)CCC[C@H]1CCC2C3CC=C4C[C@@H](O)CC[C@@H]4C3CC[C@@]21C ; CC(C)(O)CCC(C)(C)[C@H]1CCC2C3CC=C4C[C@@H](O)CC[C@@H]4C3CC[C@@]21C ; CC(CCC1(O)CC1)[C@H]1CCC2C3CC=C4C[C@@H](O)CC[C@@H]4C3CC[C@@]21C ; CC(C)(O)CCC(Cn1ccnc1)[C@H]1CCC2C3CC=C4C[C@@H](O)CC[C@@H]4C3CC[C@@]21C ; CC(O)CCC(C)[C@H]1CCC2C3CC=C4C[C@@H](O)CC[C@@H]4C3CC[C@@]21C ; CC(CCCO)[C@H]1CCC2C3CC=C4C[C@@H](O)CC[C@@H]4C3CC[C@@]21C]

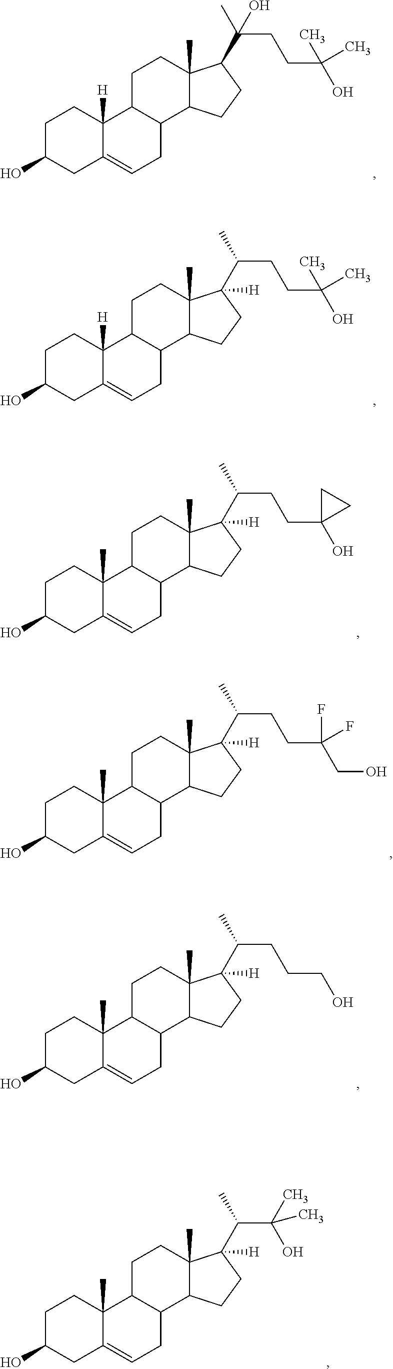 [SMILES: CC(C)(O)CCC(C)(O)[C@H]1CCC2C3CC=C4C[C@@H](O)CC[C@@H]4C3CC[C@@]21C ; C[C@H](CCC(C)(C)O)[C@H]1CCC2C3CC=C4C[C@@H](O)CC[C@@H]4C3CC[C@@]21C ; C[C@H](CCC1(O)CC1)[C@H]1CCC2C3CC=C4C[C@@H](O)CC[C@]4(C)C3CC[C@@]21C ; C[C@H](CCC(F)(F)CO)[C@H]1CCC2C3CC=C4C[C@@H](O)CC[C@]4(C)C3CC[C@@]21C ; C[C@H](CCCO)[C@H]1CCC2C3CC=C4C[C@@H](O)CC[C@]4(C)C3CC[C@@]21C ; C[C@@H]([C@H]1CCC2C3CC=C4C[C@@H](O)CC[C@]4(C)C3CC[C@@]21C)C(C)(C)O]

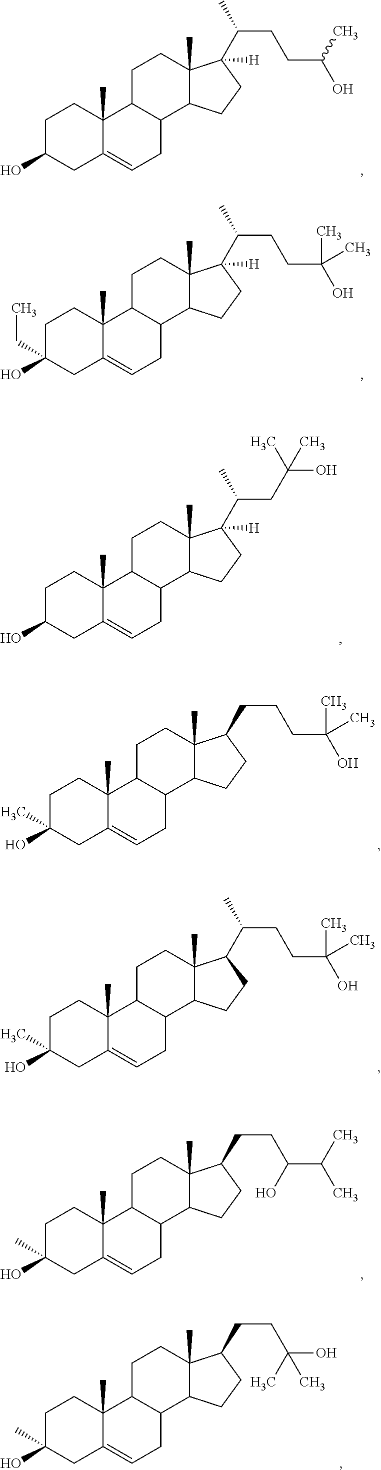

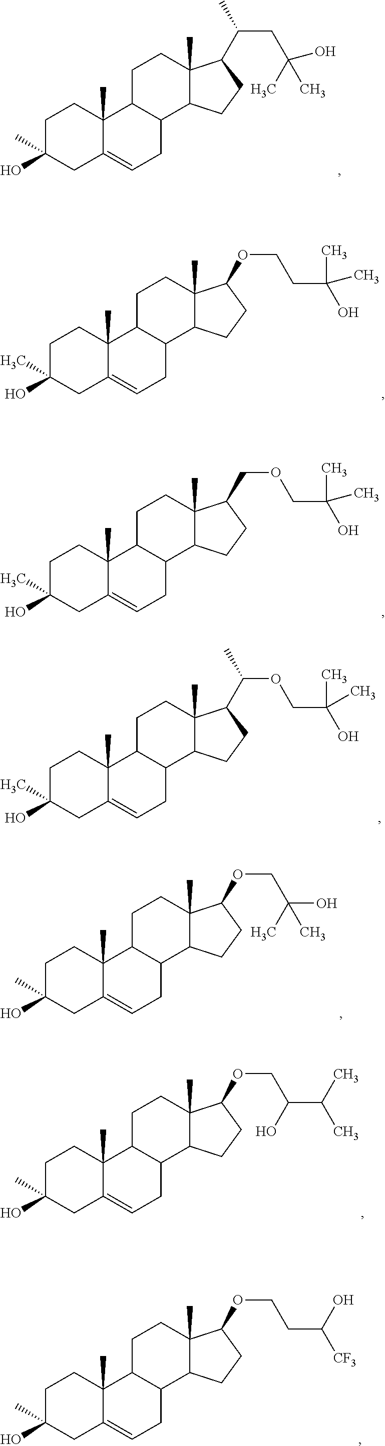

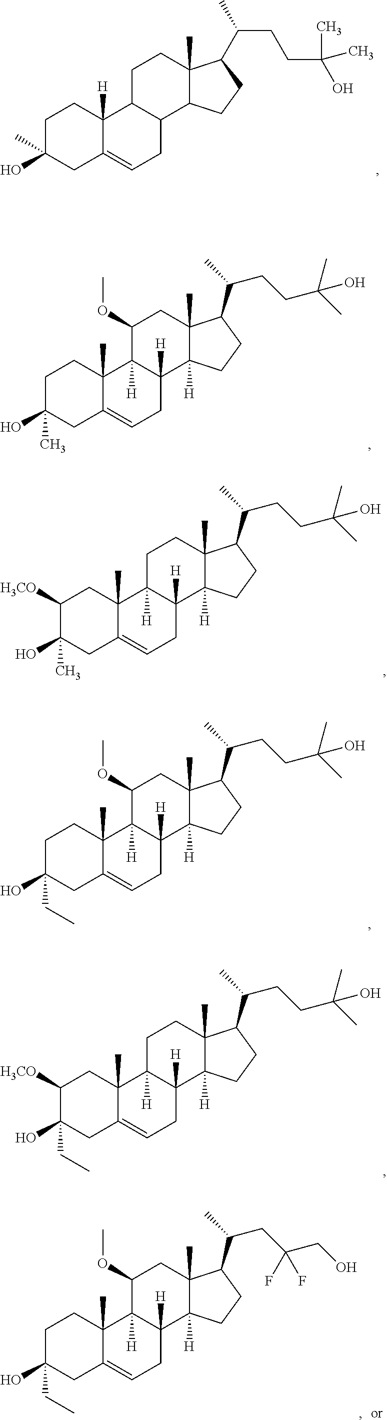

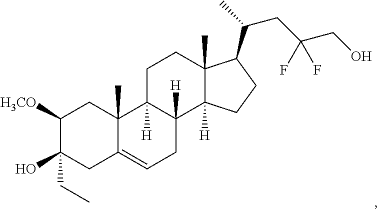

or a pharmaceutically acceptable salt, solvate, prodrug, stereoisomer, tautomer, isotopic variant, or N-oxide thereof, or a combination thereof.

In certain embodiments the compound is any one of the following compounds:

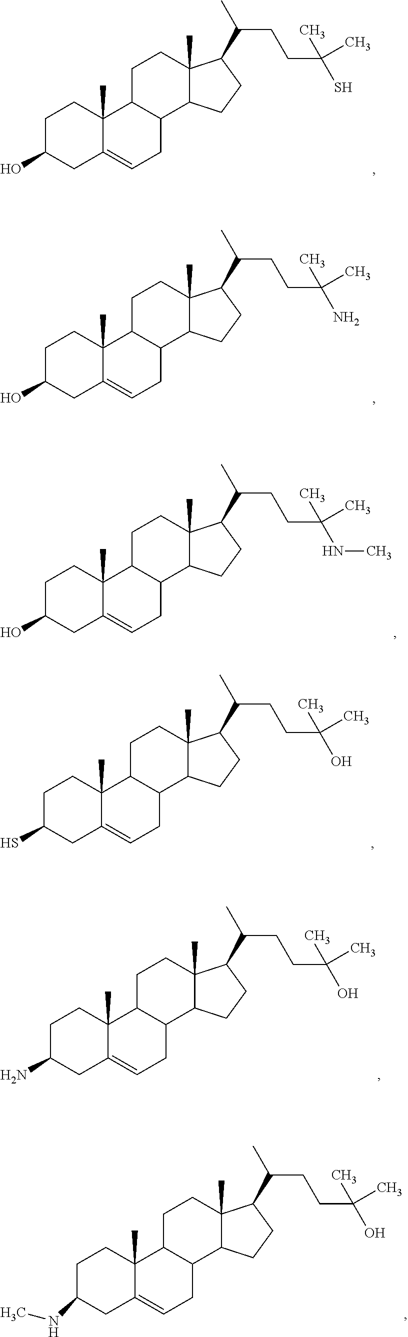

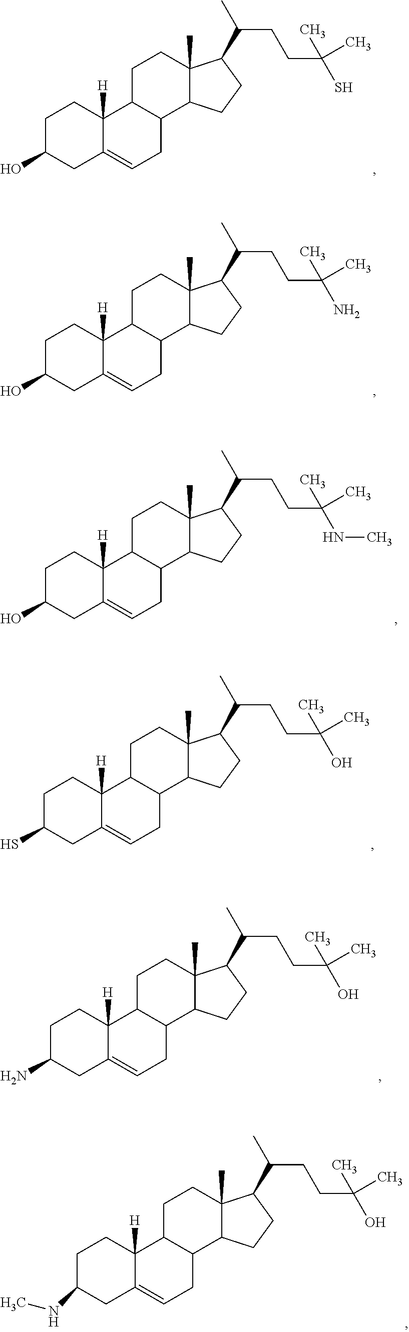

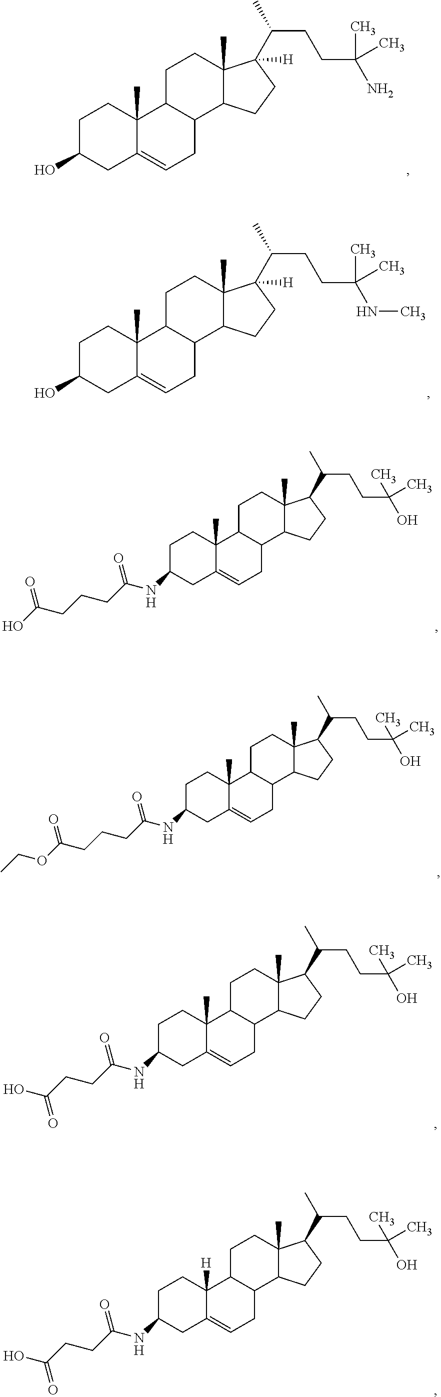

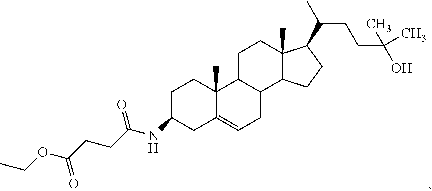 [SMILES: CCOC(=O)CCC(=O)N[C@H]1CC[C@@]2(C)C(=CCC3C2CC[C@@]2(C)C3CC[C@@H]2C(C)CCC(C)(C)O)C1]

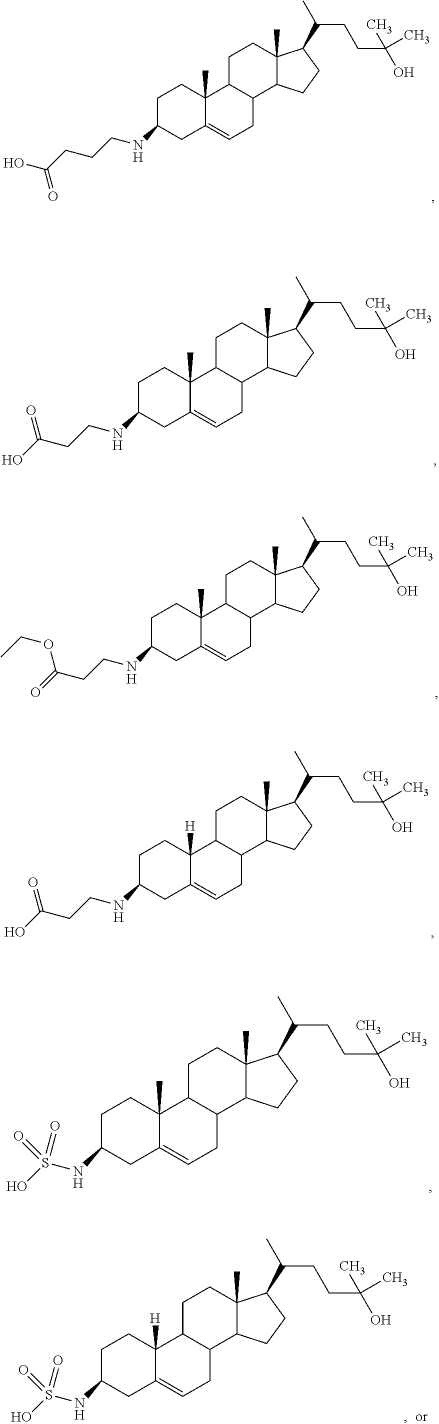

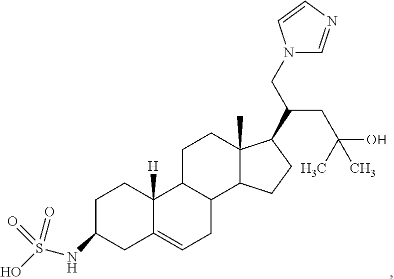 [SMILES: CC(C)(O)CC(Cn1ccnc1)[C@H]1CCC2C3CC=C4C[C@@H](NS(=O)(=O)O)CC[C@@H]4C3CC[C@@]21C]

or a pharmaceutically acceptable salt, solvate, prodrug, stereoisomer, tautomer, isotopic variant, or N-oxide thereof, or a combination thereof.

In certain embodiments the compound is any one of the following compounds:

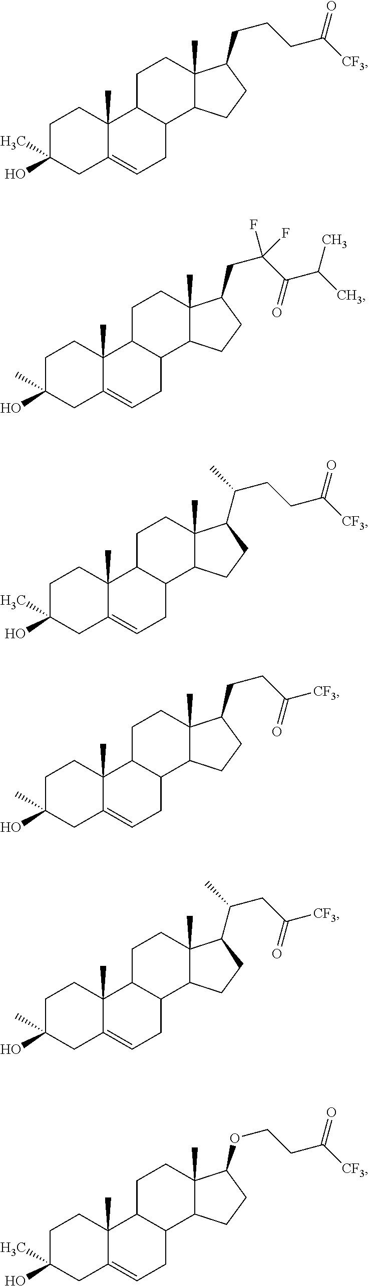

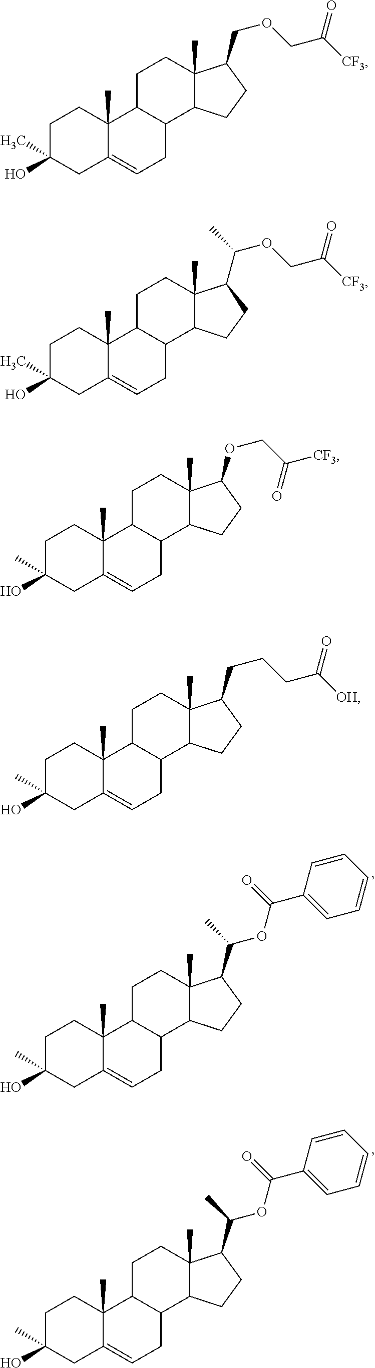

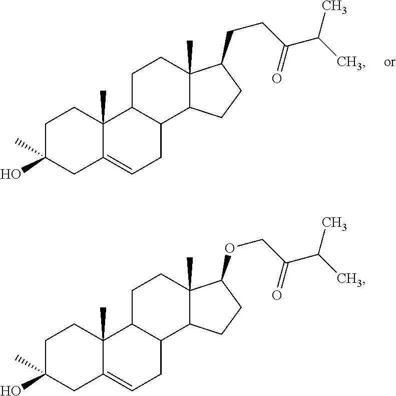 [SMILES: CC(C)C(=O)CC[C@H]1CCC2C3CC=C4C[C@@](C)(O)CC[C@]4(C)C3CC[C@@]21C ; "or" ; CC(C)C(=O)CO[C@H]1CCC2C3CC=C4C[C@@](C)(O)CC[C@]4(C)C3CC[C@@]21C]

or a pharmaceutically acceptable salt, solvate, prodrug, stereoisomer, tautomer, isotopic variant, or N-oxide thereof, or a combination thereof.

In certain embodiments the compound is any one of the following compounds:

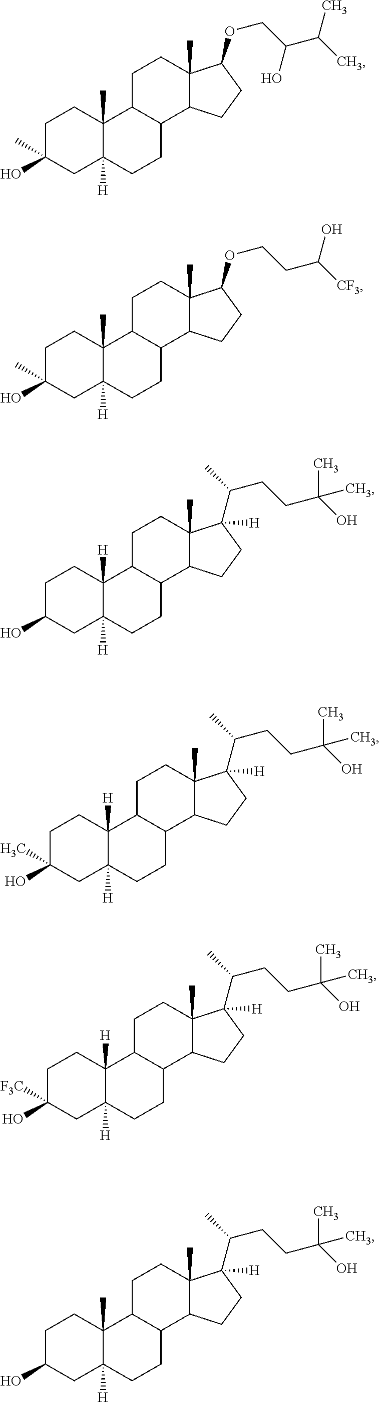 [SMILES: CC(C)C(O)CO[C@H]1CCC2C3CC[C@H]4C[C@@](C)(O)CC[C@]4(C)C3CC[C@@]21C ; C[C@]1(O)CC[C@]2(C)C3CC[C@@]4(C)C(CC[C@@H]4OCCC(O)C(F)(F)F)C3CC[C@H]2C1 ; C[C@H](CCC(C)(C)O)[C@H]1CCC2C3CC[C@H]4C[C@@H](O)CC[C@@H]4C3CC[C@@]21C ; C[C@H](CCC(C)(C)O)[C@H]1CCC2C3CC[C@H]4C[C@@](C)(O)CC[C@@H]4C3CC[C@@]21C ; C[C@H](CCC(C)(C)O)[C@H]1CCC2C3CC[C@H]4C[C@](O)(C(F)(F)F)CC[C@@H]4C3CC[C@@]21C ; C[C@H](CCC(C)(C)O)[C@H]1CCC2C3CC[C@H]4C[C@@H](O)CC[C@]4(C)C3CC[C@@]21C]

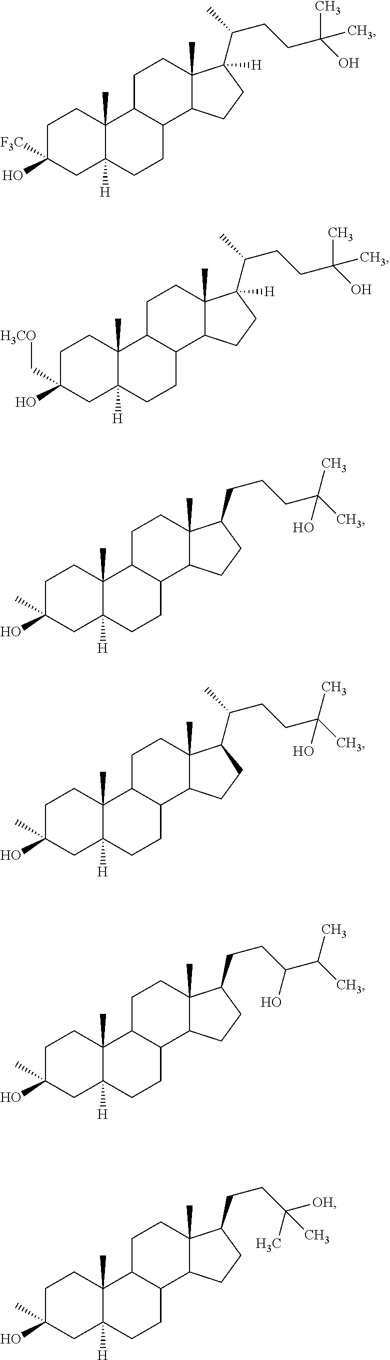

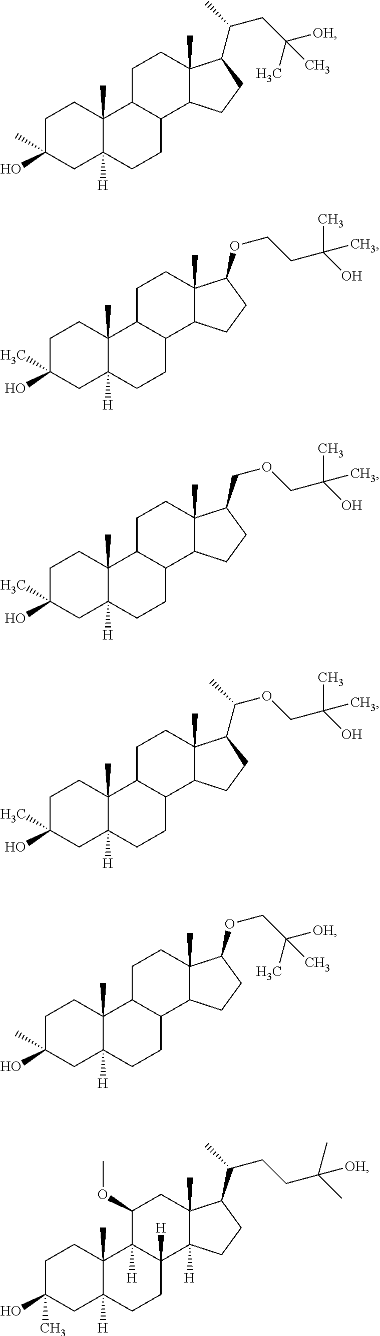

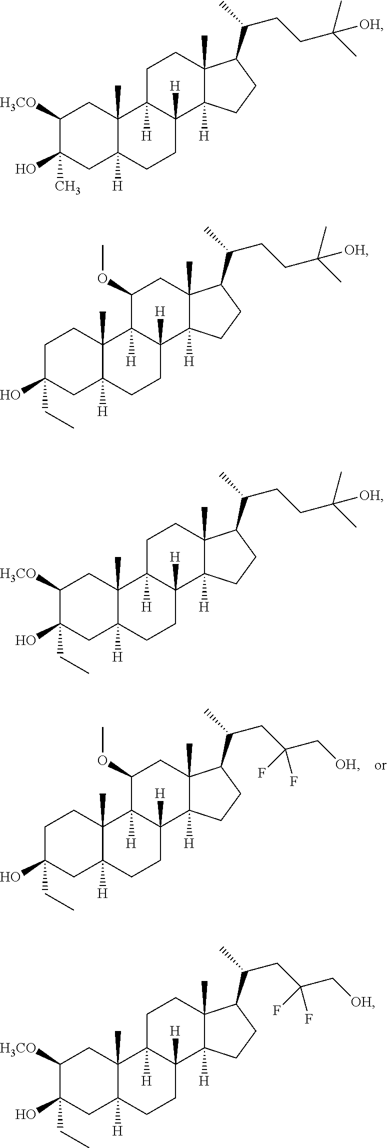

or a pharmaceutically acceptable salt, solvate, prodrug, stereoisomer, tautomer, isotopic variant, or N-oxide thereof, or a combination thereof.

In certain embodiments the compound is any one of the following compounds:

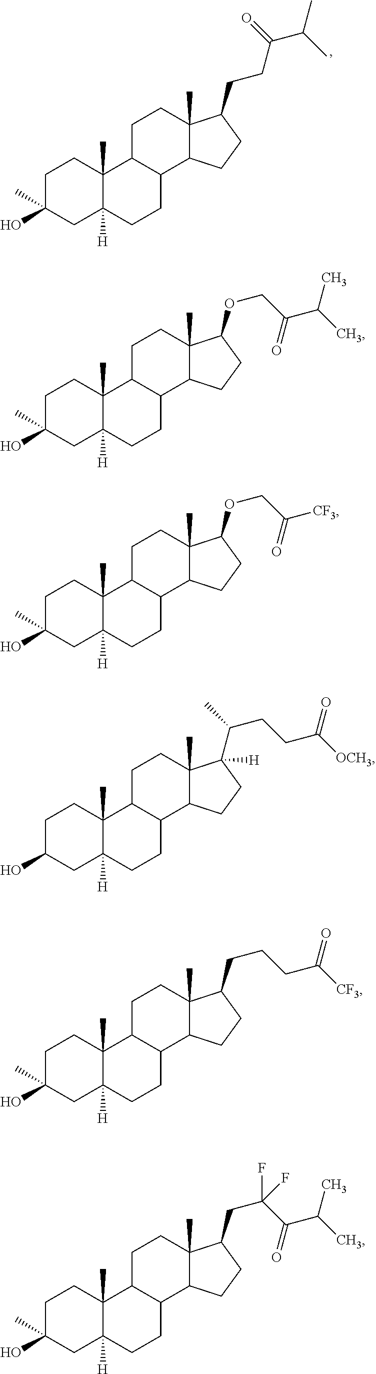 [SMILES: CC(C)C(=O)CC[C@H]1CCC2C3CC[C@H]4C[C@@](C)(O)CC[C@]4(C)C3CC[C@@]21C ; CC(C)C(=O)CO[C@H]1CCC2C3CC[C@H]4C[C@@](C)(O)CC[C@]4(C)C3CC[C@@]21C ; C[C@]1(O)CC[C@]2(C)C3CC[C@@]4(C)C(CC[C@@H]4OCC(=O)C(F)(F)F)C3CC[C@H]2C1 ; COC(=O)CC[C@@H](C)[C@H]1CCC2C3CC[C@H]4C[C@@H](O)CC[C@]4(C)C3CC[C@@]21C ; C[C@]1(O)CC[C@]2(C)C3CC[C@@]4(C)C(CC[C@@H]4CCCC(=O)C(F)(F)F)C3CC[C@H]2C1 ; CC(C)C(=O)C(F)(F)C[C@H]1CCC2C3CC[C@H]4C[C@@](C)(O)CC[C@]4(C)C3CC[C@@]21C]

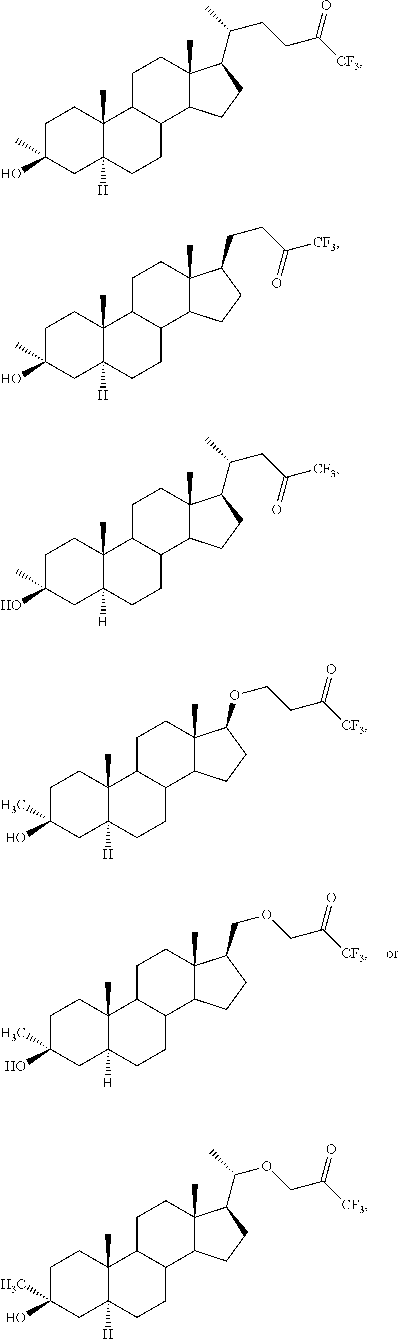

or a pharmaceutically acceptable salt, solvate, prodrug, stereoisomer, tautomer, isotopic variant, or N-oxide thereof, or a combination thereof.

In certain embodiments the compound is any one of the following compounds:

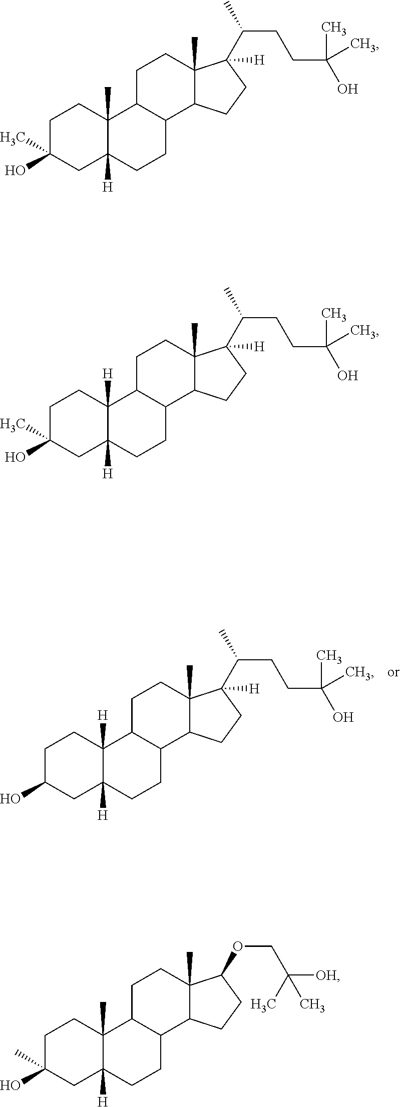 [SMILES: C[C@H](CCC(C)(C)O)[C@H]1CCC2C3CC[C@@H]4C[C@@](C)(O)CC[C@]4(C)C3CC[C@@]21C ; C[C@H](CCC(C)(C)O)[C@H]1CCC2C3CC[C@@H]4C[C@@](C)(O)CC[C@@H]4C3CC[C@@]21C ; C[C@H](CCC(C)(C)O)[C@H]1CCC2C3CC[C@@H]4C[C@@H](O)CC[C@@H]4C3CC[C@@]21C ; CC(C)(O)CO[C@H]1CCC2C3CC[C@@H]4C[C@@](C)(O)CC[C@]4(C)C3CC[C@@]21C]

or a pharmaceutically acceptable salt, solvate, prodrug, stereoisomer, tautomer, isotopic variant, or N-oxide thereof, or a combination thereof.

In certain embodiments the compound is any one of the following compounds:

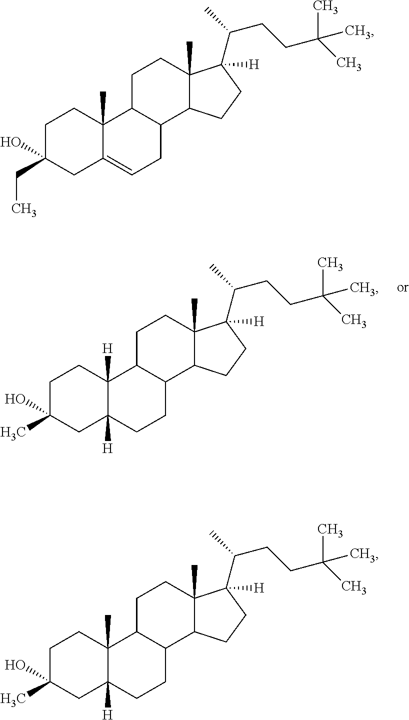 [SMILES: CC[C@@]1(O)CC[C@@]2(C)C(=CCC3C2CC[C@@]2(C)C3CC[C@@H]2[C@H](C)CCC(C)(C)C)C1 ; C[C@H](CCC(C)(C)C)[C@H]1CCC2C3CC[C@@H]4C[C@](C)(O)CC[C@@H]4C3CC[C@@]21C ; "or" ; C[C@H](CCC(C)(C)C)[C@H]1CCC2C3CC[C@@H]4C[C@](C)(O)CC[C@]4(C)C3CC[C@@]21C]

or a pharmaceutically acceptable salt, solvate, prodrug, stereoisomer, tautomer, isotopic variant, or N-oxide thereof, or a combination thereof.

In certain embodiments, the compound is a compound of Formula (I) is a compound of Formula (I-q),

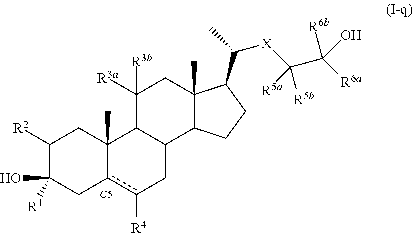

and pharmaceutically acceptable salts thereof;
wherein:
R¹ is substituted or unsubstituted aliphatic;
R² is hydrogen, halogen, substituted or unsubstituted C₁₋₆alkyl, substituted or unsubstituted cyclopropyl, or —OR^{A2}, wherein R^{A2} is hydrogen or substituted or unsubstituted alkyl;
R^{3a} is hydrogen or —OR^{A1}, wherein R^{A3} is hydrogen or substituted or unsubstituted alkyl, and R^{3b} is hydrogen; or R^{3a} and R^{3b} are joined to form an oxo (═O) group;
R⁴ is hydrogen, substituted or unsubstituted alkyl, or halogen;
X is —C(R^{X})₂— or —O—, wherein R^{X} is hydrogen or fluorine, or one R^{X} group and R^{5b} are joined to form a double bond;
each instance of R^{5a} and R^{5b} is independently hydrogen or fluorine;
R^{6a} is a non-hydrogen group selected from the group consisting of substituted and unsubstituted alkyl, substituted and unsubstituted alkenyl, substituted and unsubstituted alkynyl, substituted and unsubstituted carbocyclyl, substituted and unsubstituted heterocyclyl, substituted and unsubstituted aryl, and substituted and unsubstituted heteroaryl group, wherein the non-hydrogen group is optionally substituted with fluorine; and
R^{6b} is hydrogen or a substituted or unsubstituted alkyl group optionally substituted with fluorine;
[symbol] represents a single or double bond, provided if a single bond is present, then the hydrogen at C5 is in the alpha configuration;
   and further provided that:
(1) at least one of R^{X}, R^{5a}, and R^{5b} is fluorine; or
(2) at least one of R^{6a} and R^{6b} is a non-hydrogen group substituted with a fluorine; or
(3) R^{6a} is a non-hydrogen group comprising between two and ten carbon atoms.

In certain embodiments, the compound of the present invention is a pharmaceutically acceptable salt.

As generally described herein, compounds of formula (I-q) wherein the hydrogen at C₅ is provided in the beta configuration demonstrate loss of NMDA potentiation compared to compounds wherein the hydrogen at C₅ is alpha, or wherein a double bond is present at C₅-C₆. Thus, the compound of Formula (I-q) encompasses only compounds of Formula (I-qA) and (I-qB):

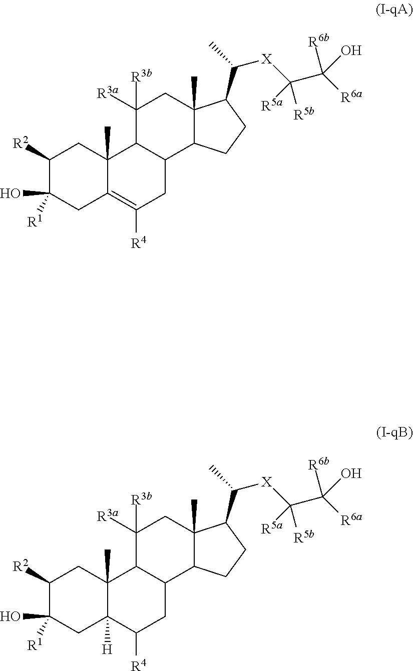

and pharmaceutically acceptable salts thereof.

### Group R¹ of Compounds of Formula (I-q)

As generally defined herein, R¹ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R¹ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R¹ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆ alkyl substituted with 1, 2, 3, 4, 5, 6, 7, 8, 9, 10, or more chloro groups (e.g., —CH₂Cl, —CHCl₂), and C₁₋₆ alkyl substituted with alkoxy groups (e.g., —CH₂OCH₃, —CH₂OCH₂CH₃, —CH₂O-cyclopropyl). In certain embodiments, R¹ is substituted alkyl, e.g., R¹ is haloalkyl, alkoxyalkyl, or aminoalkyl. In certain embodiments, R¹ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R¹ is unsubstituted C₁₋₃ alkyl, e.g., R¹ is —CH₃, —CH₂CH₃, —CH₂CH₂CH₃ or —CH₂CH₂CH₂CH₃.

In certain embodiments, R¹ is alkyl substituted with one or more fluorine atoms; e.g., le is —CH₂F, —CHF₂, or —CF₃.

In certain embodiments, R¹ is alkyl substituted with one or more —OR^{A1} groups, wherein R^{A1} is hydrogen or substituted or unsubstituted alkyl. In certain embodiments, R¹ is —CH₂OR^{A1}, e.g., wherein R^{A1} is hydrogen, —CH₃, —CH₂CH₃, or —CH₂CH₂CH₃.

In certain embodiments, R¹ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl. In certain embodiments, R¹ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R¹ is ethenyl, propenyl, or butenyl, unsubstituted or substituted with alkyl, halo, haloalkyl, alkoxyalkyl, or hydroxy. In certain embodiments, R¹ is ethenyl.

In certain embodiments, R¹ is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl. Exemplary substituted or unsubstituted R¹ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R¹ is selected from the group consisting of trifluoroethenyl, cyclopropylethynyl, cyclobutylethynyl, and propynyl, fluoropropynyl, and chloroethynyl. In certain embodiments, R¹ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R¹ is ethynyl (C₂), propynyl (C₃), or butynyl (C₄) substituted with substituted phenyl. In certain embodiments, the phenyl substitutent is further substituted with one or more substituents selected from the group consisting of halo, alkyl, trifluoroalkyl, alkoxy, acyl, amino or amido. In certain embodiments, R¹ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R¹ is ethynyl, propynyl, or butynyl, unsubstituted or substituted with alkyl, halo, haloalkyl, alkoxyalkyl, or hydroxyl. In certain embodiments, R¹ is ethynyl or propynyl, substituted with substituted or unsubstituted aryl. In certain embodiments, R¹ is ethynyl or propynyl, substituted with phenyl unsubstituted or substituted with halo, alkyl, alkoxy, haloalkyl, trihaloalkyl, or acyl. In certain embodiments, R¹ is ethynyl or propynyl, substituted with substituted or unsubstituted carbocyclyl. In certain embodiments, R^{3a} is ethynyl or propynyl, substituted with substituted or unsubstituted cyclopropyl, cyclobutyl, cyclopentyl, or cyclohexyl. In certain embodiments, R¹ is ethynyl or propynyl, substituted with substituted or unsubstituted heteroaryl. In certain embodiments, R¹ is ethynyl or propynyl, substituted with substituted or unsubstituted pyridinyl, or pyrimidinyl. In certain embodiments, R¹ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R¹ is ethynyl or propynyl, substituted with substituted or unsubstituted heterocyclyl. In certain embodiments, R¹ is ethynyl or propynyl, substituted with substituted or unsubstituted pyrrolidinyl, piperidinyl, piperazinyl, or morpholinyl. In certain embodiments, R¹ is propynyl or butynyl, substituted with hydroxyl or alkoxy. In certain embodiments, R¹ is propynyl or butynyl, substituted with methoxy or ethoxy. In certain embodiments, R¹ is ethynyl or propynyl, substituted with chloro. In certain embodiments, R¹ is ethynyl or propynyl, substituted with trifluoromethyl.

In certain embodiments, R¹ is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl. In certain embodiments, R¹ is substituted or unsubstituted cyclopropyl or substituted or unsubstituted cyclobutyl.

Groups R², R^{3a}, R^{3b}, and R⁴ of Compounds of Formula (I-q)

As generally defined herein, R² is hydrogen, halogen, substituted or unsubstituted C₁₋₆alkyl, substituted or unsubstituted cyclopropyl, or —OR^{A2}, wherein R^{A2} is hydrogen or substituted or unsubstituted alkyl. In certain embodiments, R² is hydrogen. In certain embodiments, R² is halogen, e.g., fluoro, chloro, bromo, or iodo. In certain embodiments, R² is fluoro or chloro. In certain embodiments, R² is substituted or unsubstituted C₁₋₆alkyl, e.g., substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R² is —CH₃, —CH₂CH₃, —CH₂CH₂CH₃, or cyclopropyl. In certain embodiments, R² is —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R^{A2} is hydrogen, —CH₃, —CH₂CH₃, or —CH₂CH₂CH₃, i.e., to provide a group R² of formula —OH, —OCH₃, —OCH₂CH₃, or —OCH₂CH₂CH₃. In certain embodiments, R² is a non-hydrogen substitutent in the alpha configuration. In certain embodiments, R² is a non-hydrogen substituent in the beta configuration.

As generally defined herein, R^{3a} is hydrogen or —OR^{A3}, wherein R^{A3} is hydrogen or substituted or unsubstituted alkyl, and R^{3b} is hydrogen; or R^{3a} and R^{3b} are joined to form an oxo (═O) group.

In certain embodiments, both R^{3a} and R^{3b} are both hydrogen.

In certain embodiments, R^{3a} and R^{3b} are joined to form an oxo (═O) group.

In certain embodiments, R^{3a} is —OR^{A3} and R^{3b} is hydrogen. In certain embodiments, wherein R^{3a} is —OR^{A3}, R^{3a} is in the alpha or beta configuration. In certain embodiments, wherein R^{3a} is —OR^{A3}, R^{3a} is in the alpha configuration. In certain embodiments, wherein R^{3a} is —OR^{A3}, R^{3a} is in the beta configuration. In certain embodiments, R^{A3} is hydrogen. In certain embodiments, R^{A3} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R^{A3} is hydrogen, —CH₃, —CH₂CH₃, or —CH₂CH₂CH₃, i.e., to provide a group R^{3a} of formula —OH, —OCH₃, —OCH₂CH₃, or —OCH₂CH₂CH₃.

As generally defined herein, R⁴ is hydrogen, substituted or unsubstituted alkyl, or halogen. In certain embodiments, R⁴ is hydrogen. In certain embodiments, R⁴ is halogen, e.g., fluoro. In certain embodiments, R⁴ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R⁴ is C₁ alkyl, e.g., —CH₃ or —CF₃. In certain embodiments, R⁴ is hydrogen, —CH₃, or —F. In certain embodiments, wherein [symbol] represents a single bond, R⁴ is a non-hydrogen substitutent in the alpha configuration. In certain embodiments, wherein [symbol] represents a single bond, R⁴ is a non-hydrogen substituent in the beta configuration.

Group X, R^{5a}, R^{5b}, R^{6a}, and R^{6b} of Compounds of Formula (I-q)

As generally defined herein, X is —C(R^{X})₂— or —O—, wherein R^{X} is hydrogen or fluorine, or one R^{X} group and R^{5b} are joined to form a double bond; each of R^{5a} and R^{5b} is independently hydrogen or fluorine; R^{6a} is a non-hydrogen group selected from the group consisting of substituted and unsubstituted alkyl, substituted and unsubstituted alkenyl, substituted and unsubstituted alkynyl, substituted and unsubstituted carbocyclyl, substituted and unsubstituted heterocyclyl, substituted and unsubstituted aryl, and substituted and unsubstituted heteroaryl group, wherein the non-hydrogen group is optionally substituted with fluorine; and R^{6b} is hydrogen or a substituted or unsubstituted alkyl group optionally substituted with fluorine; provided: (1) at least one of R^{X}, R^{5a}, and R^{5b} is fluorine; or (2) at least one of R^{6a} and R^{6b} is a non-hydrogen group substituted with fluorine; or (3) R^{6a} is a non-hydrogen group comprising between two and ten carbon atoms.

In certain embodiments, X is —O—. In certain embodiments, X is —CH₂—. In certain embodiments, X is —CF₂—.

In certain embodiments, at least one of R^{5a} and R^{5b} is hydrogen. In certain embodiments, at least one of R^{5a} and R^{5b} is fluorine. In certain embodiments, R^{5a} and R^{5b} are both hydrogen. In certain embodiments, R^{5a} and R^{5b} are both fluorine. In certain embodiments, R^{X} and R^{5b} are joined to form a double bond, e.g., cis or trans double bond.

In certain embodiments, R^{6a} is a non-hydrogen group, as described herein, which is not substituted with fluorine. In certain embodiments, R^{6a} is substituted or unsubstituted alkyl (e.g., —CH₃, —CH₂CH₃, —CH(CH₃)₂), substituted or unsubstituted alkenyl, substituted or unsubstituted alkynyl, or substituted or unsubstituted carbocyclyl (e.g., isopropanol). In certain embodiments, R^{6a} is a non-hydrogen group, as described herein, which is substituted with fluorine.

In certain embodiments, R^{6a} is a non-hydrogen group, as described herein, and R^{6b} is hydrogen. In certain embodiments, R^{6a} is a non-hydrogen group, as described herein, and R^{6b} is a substituted or unsubstituted alkyl group optionally substituted by fluorine. In certain embodiments, R^{6b} is an alkyl group which is not substituted with fluorine. In certain embodiments, R^{6a} is an alkyl group which is substituted with fluorine.

In certain embodiments, R^{6b} is hydrogen. In certain embodiments, R^{6b} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl, optionally substituted by fluorine. In certain embodiments, R^{6b} is C₁ alkyl optionally substituted by fluorine, e.g., —CH₃ or —CF₃.

In certain embodiments, R^{6a} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R^{6a} C₁₋₆alkyl groups include, but are not limited to, substituted or unsubstituted methyl (C₁), substituted or unsubstituted ethyl (C₂), substituted or unsubstituted n-propyl (C₃), substituted or unsubstituted isopropyl (C₃), substituted or unsubstituted n-butyl (C₄), substituted or unsubstituted tert-butyl (C₄), substituted or unsubstituted sec-butyl (C₄), substituted or unsubstituted iso-butyl (C₄), substituted or unsubstituted n-pentyl (C₅), substituted or unsubstituted 3-pentanyl (C₅), substituted or unsubstituted amyl (C₅), substituted or unsubstituted neopentyl (C₅), substituted or unsubstituted 3-methyl-2-butanyl (C₅), substituted or unsubstituted tertiary amyl (C₅), substituted or unsubstituted n-hexyl (C₆). In certain embodiments, R^{6a} is alkyl, as described above, substituted with one or more fluorines, e.g., 1, 2, 3, 4, or more fluorines. In certain embodiments, R^{6a} is —CF₃, —CH₂F, —CHF₂, difluoroethyl, or 2,2,2-trifluoro-1,1-dimethyl-ethyl). In certain embodiments, R^{6a} is alkyl, as described above, substituted with one or more —OR^{A6} groups, wherein R^{A6} is hydrogen or substituted or unsubstituted alkyl. In certain embodiments, R^{6a} is —CH₂OR^{A6}, —CH₂CH₂OR^{A6}, or —CH₂CH₂CH₂OR^{A6}, e.g., —CH₂OCH₃, —CH₂CH₂OCH₃, or —CH₂CH₂CH₂OCH₃.

In certain embodiments, R^{6a} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl, optionally substituted with fluorine. In certain embodiments, R^{6a} is substituted or unsubstituted vinyl (C₂) or substituted or unsubstituted allyl (C₃).

In certain embodiments, R^{6a} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl, optionally substituted with fluorine. In certain embodiments, R^{6a} is substituted or unsubstituted ethynyl (C₂) or substituted or unsubstituted propargyl (C₃).

In certain embodiments, R^{6a} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl, optionally substituted with fluorine. In certain embodiments, R^{6a} is substituted or unsubstituted cyclopropyl.

In certain embodiments, R^{6a} is substituted or unsubstituted heterocyclyl, e.g., substituted or unsubstituted C₃₋₆ heterocyclyl, substituted or unsubstituted C₃₋₄ heterocyclyl, substituted or unsubstituted C₄₋₅ heterocyclyl, or substituted or unsubstituted C₅₋₆ heterocyclyl, optionally substituted with fluorine.

In certain embodiments, R^{6a} is substituted or unsubstituted aryl, e.g., substituted or unsubstituted phenyl, optionally substituted with fluorine.

In certain embodiments, R^{6a} is substituted or unsubstituted heteroaryl, e.g., optionally substituted 5- to 6-membered heteroaryl, optionally substituted with fluorine.

In certain embodiments, R^{6a} is a non-hydrogen group comprising between two and ten carbon atoms, e.g., between two and nine, two and eight, two and seven, two and six, two and five, two and four, or two and three carbon atoms, inclusive. For example, in certain embodiments, R^{6a} is substituted or unsubstituted C₂₋₃ alkyl, substituted or unsubstituted C₂₋₃ alkenyl, substituted or unsubstituted C₂₋₃ alkynyl, or substituted or unsubstituted C₃ carbocyclyl.

In certain embodiments, wherein at least one of R^{X}, R^{5a}, and R^{5b} is fluorine; or at least one of R^{6a} and R^{6b} is a non-hydrogen group substituted with fluorine; R^{6a} is substituted or unsubstituted C₁₋₃ alkyl, substituted or unsubstituted C₁₋₃ alkenyl, substituted or unsubstituted C₁₋₃ alkynyl, or substituted or unsubstituted C₃ carbocyclyl.

In certain embodiments, R^{6a} and R^{6b} are the same group. In certain embodiments, R^{6a} and R^{6b} are different groups, and the carbon to R^{6a} is attached is in the (S) or (R) configuration. In certain embodiments, the carbon to which R^{6a} is attached is in the (S) configuration. In certain embodiments, the carbon to which R^{6a} is attached is in the (R) configuration. In certain embodiments, R^{6a} is —CF₃ and R^{6b} is hydrogen or C₁₋₄ alkyl. In certain embodiments, R^{6a} is a non-hydrogen group substituted with fluorine, and R^{6b} is —CH₃. In certain embodiments, R^{6a} is substituted with one or more —OR^{A6} groups, wherein R^{A6} is hydrogen or substituted or unsubstituted alkyl. In certain embodiments, R^{6a} is a substituted or unsubstituted C₂₋₄ alkyl, substituted or unsubstituted C₂₋₃ alkenyl, substituted or unsubstituted C₂₋₃ alkynyl, or substituted or unsubstituted C₃ carbocyclyl, and R^{6b} is —CH₃. In certain embodiments, R^{6a} is a unsubstituted C₂₋₄ alkyl, unsubstituted C₂₋₃ alkenyl, or unsubstituted C₂₋₃ alkynyl, or unsubstituted C₃ carbocyclyl, and R^{6b} is —CH₃. In certain embodiments, R^{6a} is a non-hydrogen group substituted with fluorine, and R^{6b} is —CH₃.

### Various Combinations of Certain Embodiments

Various combinations of certain embodiments are further contemplated herein.

For example, in certain embodiments, wherein X is —CH₂— and R^{5a} and R^{5b} are both hydrogen, provided is a compound of Formula (I-qa):

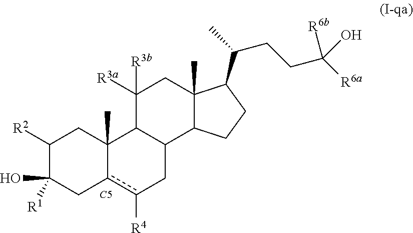

or a pharmaceutically acceptable salt thereof. In certain embodiments, R^{6a} is a non-hydrogen group comprising between two and ten carbon atoms. In certain embodiments, at least one of R^{6a} and R^{6b} is a non-hydrogen group substituted with fluorine. In certain embodiments, the carbon to which R^{6a} is attached is in the (S) configuration. In certain embodiments, the carbon to which R^{6a} is attached is in the (R) configuration. In certain embodiments, R^{6a} is methyl (C₁) optionally substituted with one or more fluorines, e.g., —CH₃ or —CF₃. In certain embodiments, R^{6a} is substituted or unsubstituted ethyl (C₂), substituted or unsubstituted n-propyl (C₃), or substituted or unsubstituted isopropyl (C₃). In certain embodiments, R^{6a} is —CH₂OR^{A6}, —CH₂CH₂OR^{A6}, or —CH₂CH₂CH₂OR^{A6}. In certain embodiments, R^{6a} is substituted or unsubstituted vinyl (C₂) or substituted or unsubstituted allyl (C₃). In certain embodiments, R^{6a} is substituted or unsubstituted ethynyl (C₂) or substituted or unsubstituted propargyl (C₃). In certain embodiments, R^{6a} is substituted or unsubstituted cyclopropyl. In certain embodiments, R^{6b} is hydrogen. In certain embodiments, R^{6b} is —CH₃ or —CF₃. In certain embodiments, [symbol] represents a single bond, and the hydrogen at C5 is alpha. In certain embodiments, [symbol] represents a double bond. In certain embodiments, R¹ is —CH₃ or —CH₂CH₃. In certain embodiments, R² is hydrogen, —OH, —OCH₃, —OCH₂CH₃, —OCH₂CH₂CH₃, —CH₃, —CH₂CH₃, —CH₂CH₂CH₃, cyclopropyl, fluoro, or chloro. In certain embodiments, R² is a non-hydrogen substitutent in the alpha configuration. In certain embodiments, R² is a non-hydrogen substituent in the beta configuration. In certain embodiments, R^{3a} and R^{3b} are both hydrogen. In certain embodiments, R^{3a} and R^{3b} are joined to form ═O (oxo). In certain embodiments, R⁴ is hydrogen.

In certain embodiments, wherein X is —CH₂— and R^{5a} and R^{5b} are both fluorine, provided is a compound of Formula (I-qb):

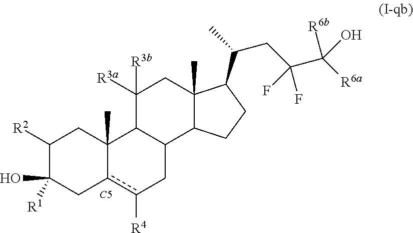

or a pharmaceutically acceptable salt thereof. In certain embodiments, R^{6a} is a non-hydrogen group comprising between two and ten carbon atoms. In certain embodiments, at least one of R^{6a} and R^{6b} is a non-hydrogen group substituted with fluorine. In certain embodiments, the carbon to which R^{6a} is attached is in the (S) configuration. In certain embodiments, the carbon to which R^{6a} is attached is in the (R) configuration. In certain embodiments, R^{6a} is methyl (C₁), optionally substituted with one or more fluorines, e.g., —CH₃ or —CF₃. In certain embodiments, R^{6a} is substituted or unsubstituted ethyl (C₂), substituted or unsubstituted n-propyl (C₃), or substituted or unsubstituted isopropyl (C₃). In certain embodiments, R^{6a} is —CH₂OR^{A6}, —CH₂CH₂OR^{A6}, or —CH₂CH₂CH₂OR^{A6}. In certain embodiments, R^{6a} is substituted or unsubstituted vinyl (C₂) or substituted or unsubstituted allyl (C₃). In certain embodiments, R^{6a} is substituted or unsubstituted ethynyl (C₂) or substituted or unsubstituted propargyl (C₃). In certain embodiments, R^{6a} is substituted or unsubstituted cyclopropyl. In certain embodiments, R^{6b} is hydrogen. In certain embodiments, R^{6b} is —CH₃ or —CF₃. In certain embodiments, [symbol] represents a single bond, and the hydrogen at C5 is alpha. In certain embodiments, [symbol] represents a double bond. In certain embodiments, R¹ is —CH₃ or —CH₂CH₃ In certain embodiments, R² is hydrogen, —OH, —OCH₃, —OCH₂CH₃, —OCH₂CH₂CH₃, —CH₃, —CH₂CH₃, —CH₂CH₂CH₃, cyclopropyl, fluoro, or chloro. In certain embodiments, R² is a non-hydrogen substitutent in the alpha configuration. In certain embodiments, R² is a non-hydrogen substituent in the beta configuration. In certain embodiments, R^{3a} and R^{3b} are both hydrogen. In certain embodiments, R^{3a} and R^{3b} are joined to form ═O (oxo). In certain embodiments, R⁴ is hydrogen.

In certain embodiments, wherein X is —C(R^{X})₂— and one R^{X} group and R^{5b} are joined to form a trans double bond, provided is a compound of Formula (I-qc):

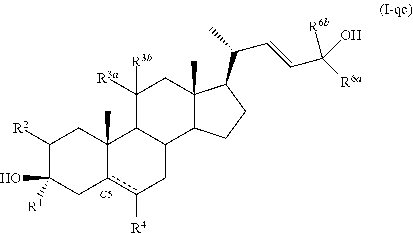

or a pharmaceutically acceptable salt thereof. In certain embodiments, R^{6a} is a non-hydrogen group comprising between two and ten carbon atoms. In certain embodiments, at least one of R^{6a} and R^{6b} is a non-hydrogen group substituted with fluorine. In certain embodiments, the carbon to which R^{6a} is attached is in the (S) configuration. In certain embodiments, the carbon to which R^{6a} is attached is in the (R) configuration. In certain embodiments, R^{6a} is methyl (C₁) optionally substituted with one or more fluorines, e.g., —CH₃ or —CF₃. In certain embodiments, R^{6a} is substituted or unsubstituted ethyl (C₂), substituted or unsubstituted n-propyl (C₃), or substituted or unsubstituted isopropyl (C₃). In certain embodiments, R^{6a} is —CH₂OR^{A6}, —CH₂CH₂OR^{A6}, —CH₂CH₂OR^{A6}, or —CH₂CH₂CH₂OR^{A6}. In certain embodiments, R^{6a} is substituted or unsubstituted vinyl (C₂) or substituted or unsubstituted allyl (C₃). In certain embodiments, R^{6a} is substituted or unsubstituted ethynyl (C₂) or substituted or unsubstituted propargyl (C₃). In certain embodiments, R^{6a} is substituted or unsubstituted cyclopropyl. In certain embodiments, R^{6b} is hydrogen. In certain embodiments, R^{6b} is —CH₃ or —CF₃. In certain embodiments, [symbol] represents a single bond, and the hydrogen at C5 is alpha. In certain embodiments, [symbol] represents a double bond. In certain embodiments, R¹ is —CH₃ or —CH₂CH₃. In certain embodiments, R² is hydrogen, —OH, —OCH₃, —OCH₂CH₃, —OCH₂CH₂CH₃, —CH₃, —CH₂CH₃, —CH₂CH₂CH₃, cyclopropyl, fluoro, or chloro. In certain embodiments, R² is a non-hydrogen substitutent in the alpha configuration. In certain embodiments, R² is a non-hydrogen substituent in the beta configuration. In certain embodiments, R^{3a} and R^{3b} are both hydrogen. In certain embodiments, R^{3a} and R^{3b} are joined to form ═O (oxo). In certain embodiments, R⁴ is hydrogen.

In certain embodiments, the compound of Formula (I-q) is selected from a compound of Formula (I-qd):

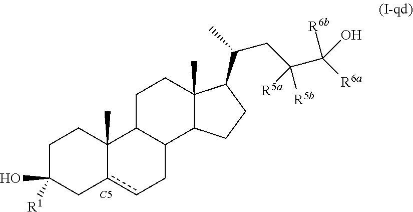

or a pharmaceutically acceptable salt thereof. In certain embodiments, R^{6a} is a non-hydrogen group comprising between two and ten carbon atoms. In certain embodiments, at least one of R^{6a} and R^{6b} is a non-hydrogen group substituted with fluorine. In certain embodiments, the carbon to which R^{6a} is attached is in the (S) configuration. In certain embodiments, the carbon to which R^{6a} is attached is in the (R) configuration. In certain embodiments, R^{6a} is methyl (C₁) optionally substituted with one or more fluorines, e.g., —CH₃ or —CF₃. In certain embodiments, R^{6a} is substituted or unsubstituted ethyl (C₂), substituted or unsubstituted n-propyl (C₃), or substituted or unsubstituted isopropyl (C₃). In certain embodiments, R^{6a} is —CH₂OR^{A6}, —CH₂CH₂OR^{A6}, or —CH₂CH₂CH₂OR^{A6}. In certain embodiments, R^{6a} is substituted or unsubstituted vinyl (C₂) or substituted or unsubstituted allyl (C₃). In certain embodiments, R^{6a} is substituted or unsubstituted ethynyl (C₂) or substituted or unsubstituted propargyl (C₃). In certain embodiments, R^{6a} is substituted or unsubstituted cyclopropyl. In certain embodiments, R^{6b} is hydrogen. In certain embodiments, R^{6b} is —CH₃ or —CF₃. In certain embodiments, [symbol] represents a single bond, and the hydrogen at C5 is alpha. In certain embodiments, [symbol] represents a double bond. In certain embodiments, R¹ is —CH₃ or —CH₂CH₃.

In certain embodiments, the compound of Formula (I-q) is selected from a compound of Formula (I-qe):

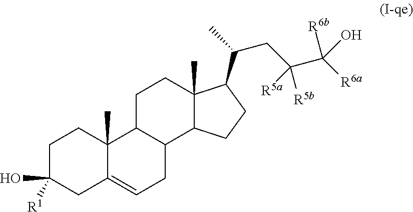

or a pharmaceutically acceptable salt thereof. In certain embodiments, R^{6a} is a non-hydrogen group comprising between two and ten carbon atoms. In certain embodiments, at least one of R^{6a} and R^{6b} is a non-hydrogen group substituted with fluorine. In certain embodiments, the carbon to which R^{6a} is attached is in the (S) configuration. In certain embodiments, the carbon to which R^{6a} is attached is in the (R) configuration. In certain embodiments, R^{6a} is methyl (C₁) optionally substituted with one or more fluorines, e.g., —CH₃ or —CF₃. In certain embodiments, R^{6a} is substituted or unsubstituted ethyl (C₂), substituted or unsubstituted n-propyl (C₃), or substituted or unsubstituted isopropyl (C₃). In certain embodiments, R^{6a} is —CH₂OR^{A6}, —CH₂CH₂OR^{A6}, CH₂CH₂OR^{A6}, or —CH₂CH₂CH₂OR^{A6}. In certain embodiments, R^{6a} is substituted or unsubstituted vinyl (C₂) or substituted or unsubstituted allyl (C₃). In certain embodiments, R^{6a} is substituted or unsubstituted ethynyl (C₂) or substituted or unsubstituted propargyl (C₃). In certain embodiments, R^{6a} is substituted or unsubstituted cyclopropyl. In certain embodiments, R^{6b} is hydrogen. In certain embodiments, R^{6b} is —CH₃ or —CF₃. In certain embodiments, R¹ is —CH₃ or —CH₂CH₃.

In certain embodiments, the compound of Formula (I-q) is selected from a compound of Formula (I-qf):

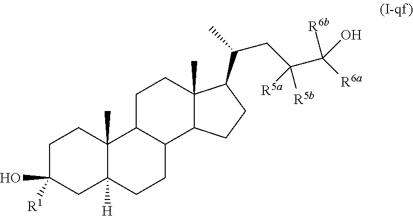

or a pharmaceutically acceptable salt thereof. In certain embodiments, R^{6a} is a non-hydrogen group comprising between two and ten carbon atoms. In certain embodiments, at least one of R^{6a} and R^{6b} is a non-hydrogen group substituted with fluorine. In certain embodiments, the carbon to which R^{6a} is attached is in the (S) configuration. In certain embodiments, the carbon to which R^{6a} is attached is in the (R) configuration. In certain embodiments, R^{6a} is methyl (C₁) optionally substituted with one or more fluorines, e.g., —CH₃ or —CF₃. In certain embodiments, R^{6a} is substituted or unsubstituted ethyl (C₂), substituted or unsubstituted n-propyl (C₃), or substituted or unsubstituted isopropyl (C₃). In certain embodiments, R^{6a} is —CH₂OR^{A6}, —CH₂CH₂OR^{A6}, CH₂CH₂OR^{A6}, or —CH₂CH₂CH₂OR^{A6}. In certain embodiments, R^{6a} is substituted or unsubstituted vinyl (C₂) or substituted or unsubstituted allyl (C₃). In certain embodiments, R^{6a} is substituted or unsubstituted ethynyl (C₂) or substituted or unsubstituted propargyl (C₃). In certain embodiments, R^{6a} is substituted or unsubstituted cyclopropyl. In certain embodiments, R^{6b} is hydrogen. In certain embodiments, R^{6b} is —CH₃ or —CF₃. In certain embodiments, R¹ is —CH₃ or —CH₂CH₃.

In certain embodiments, a compound of Formula (I-q) is selected from the group consisting of:

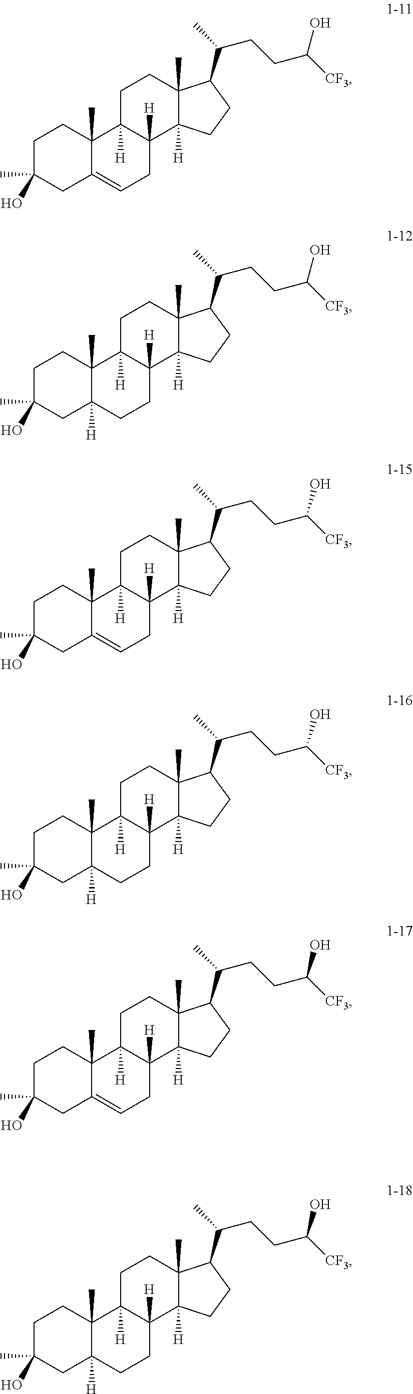

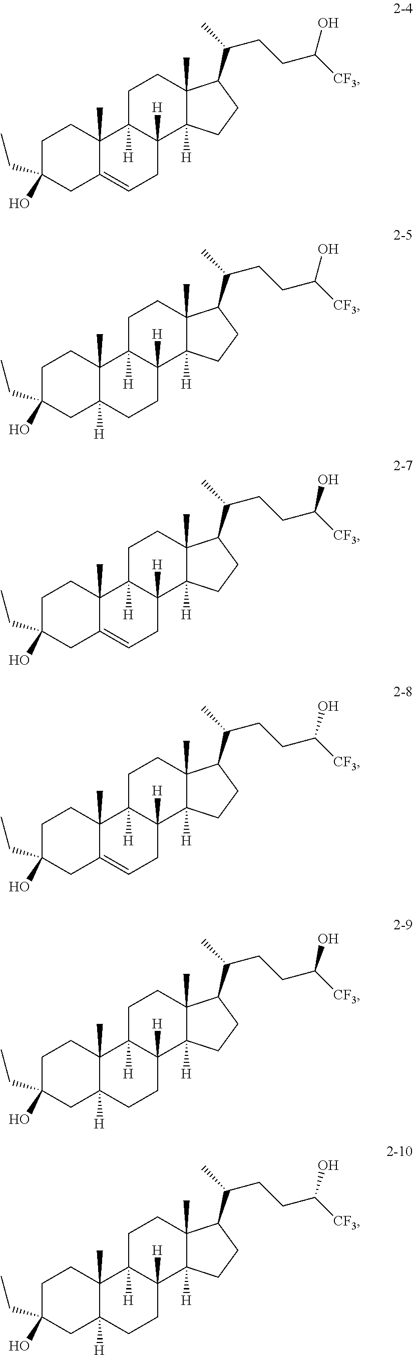

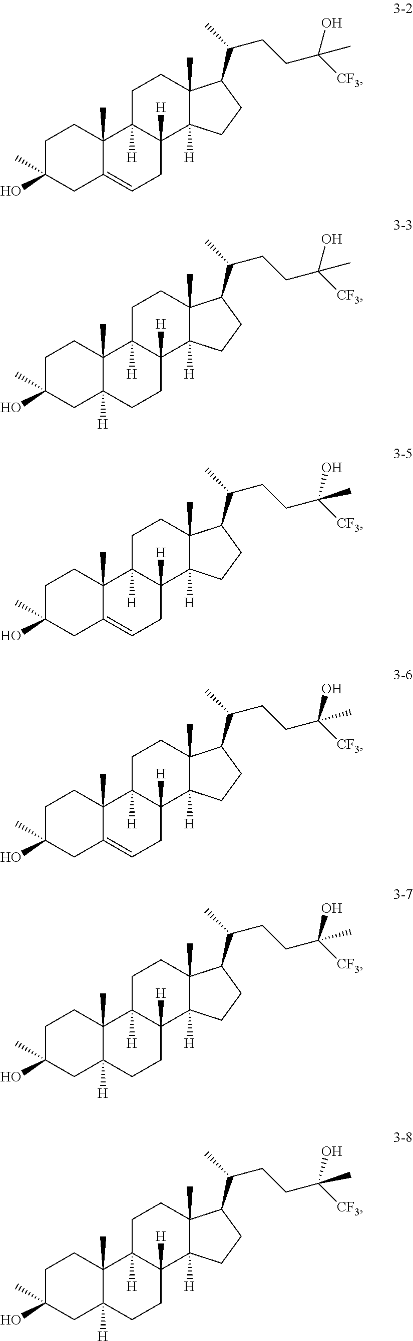

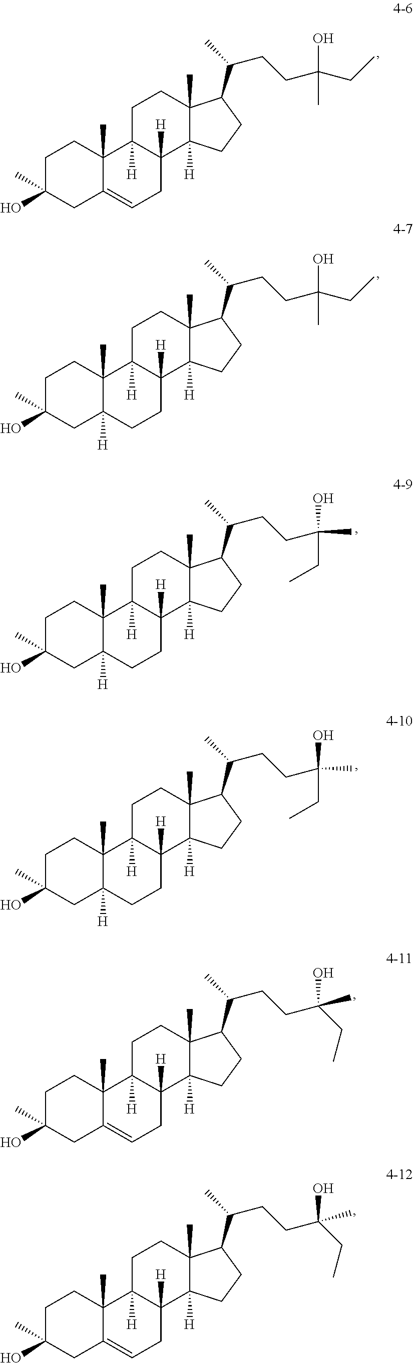

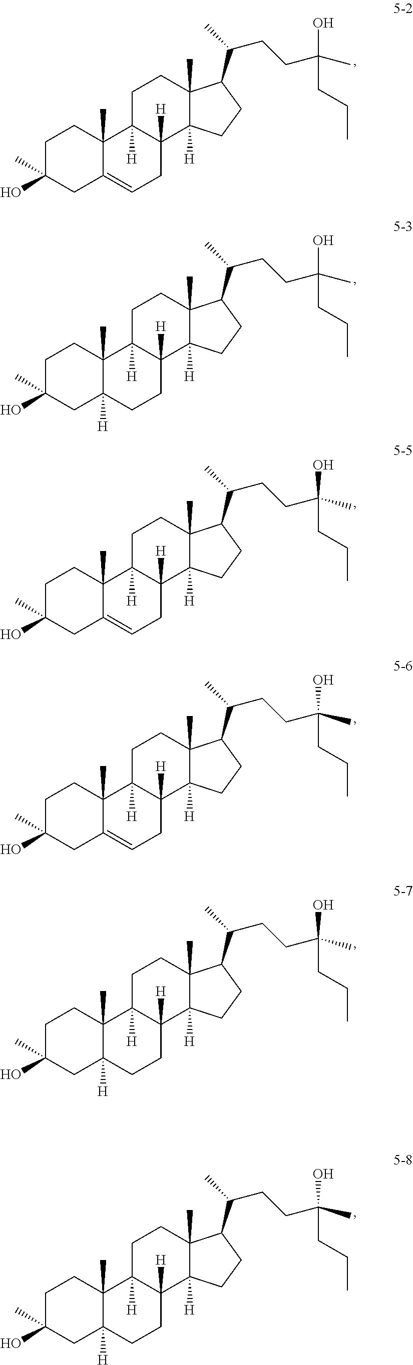

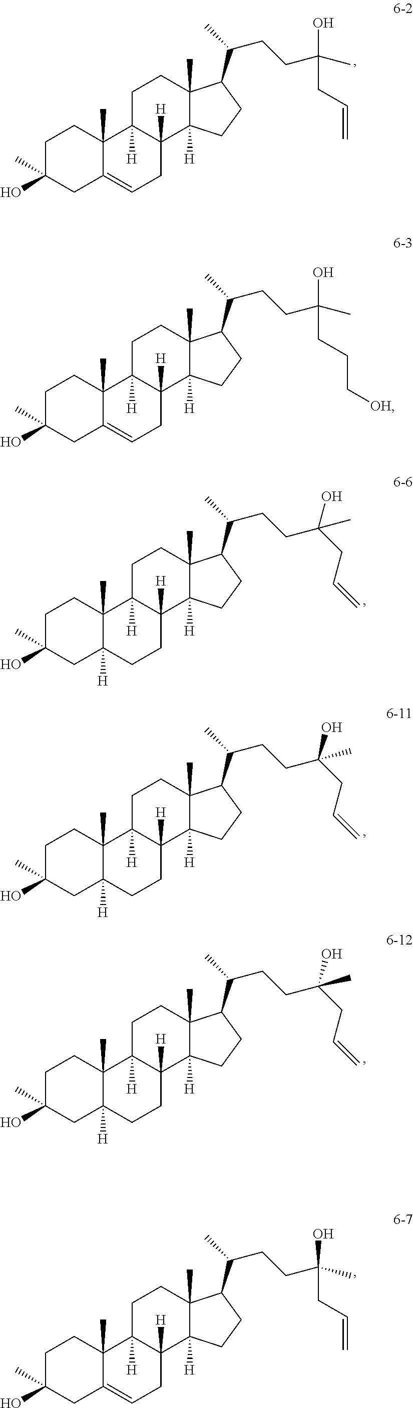

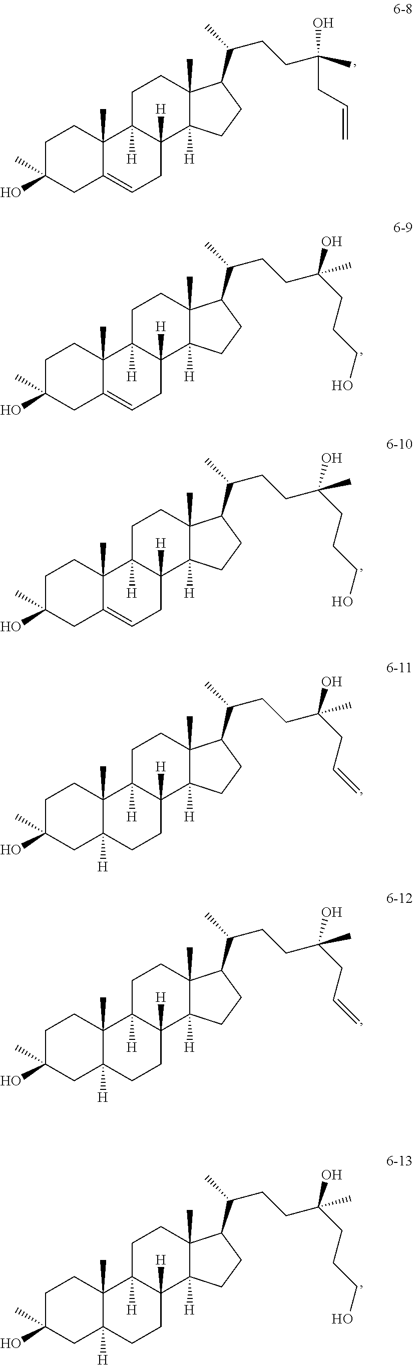

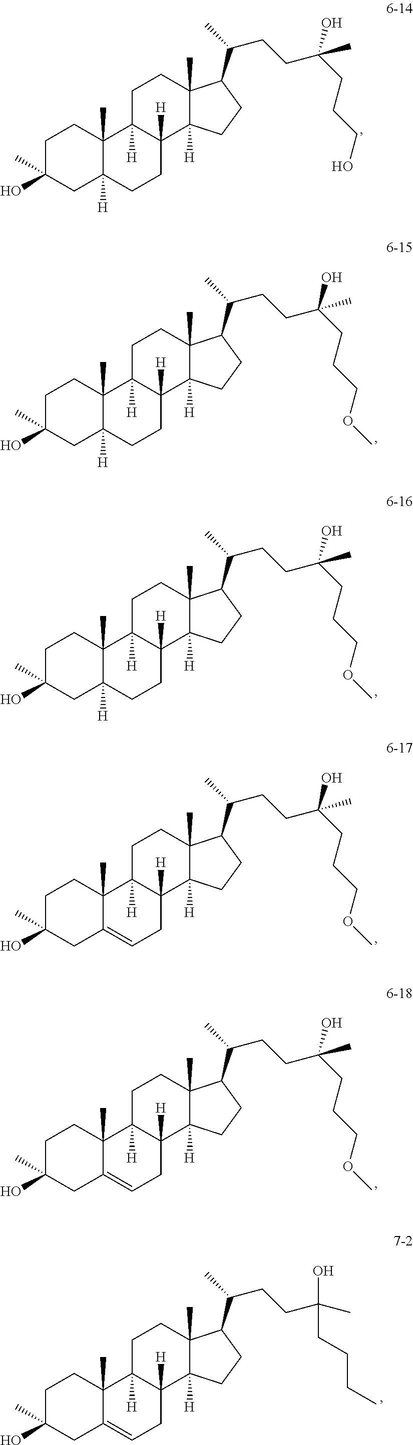

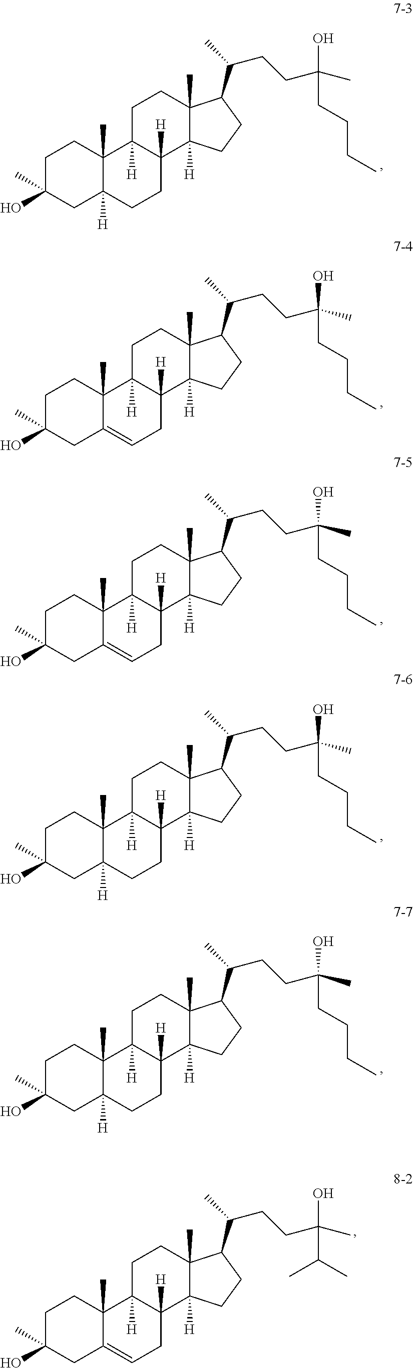

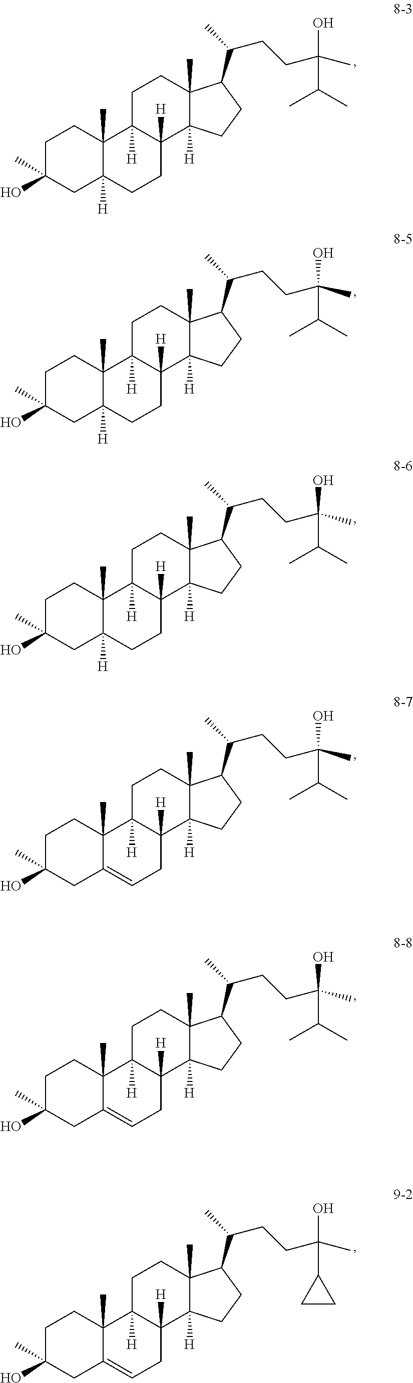

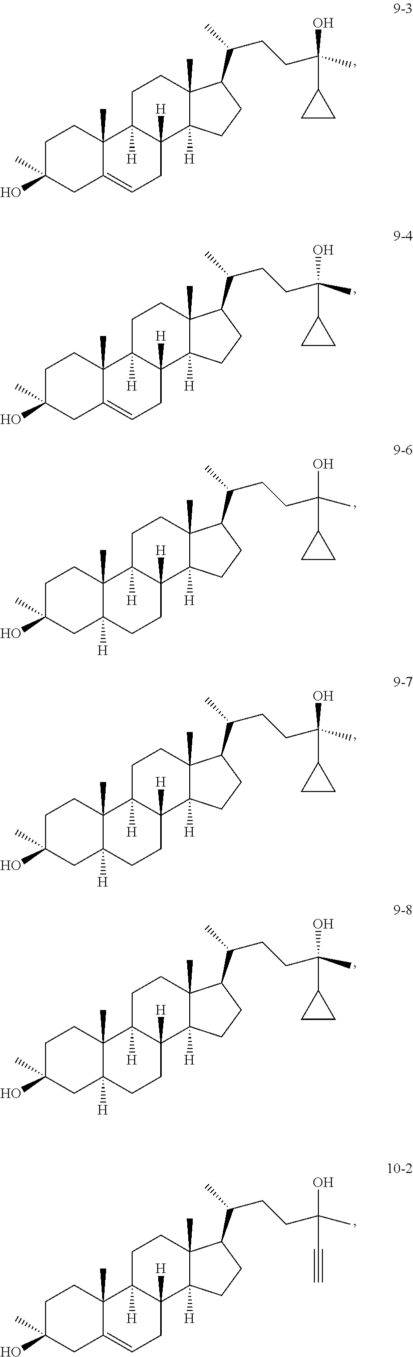

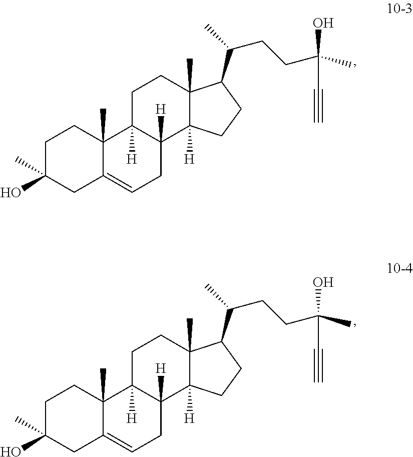

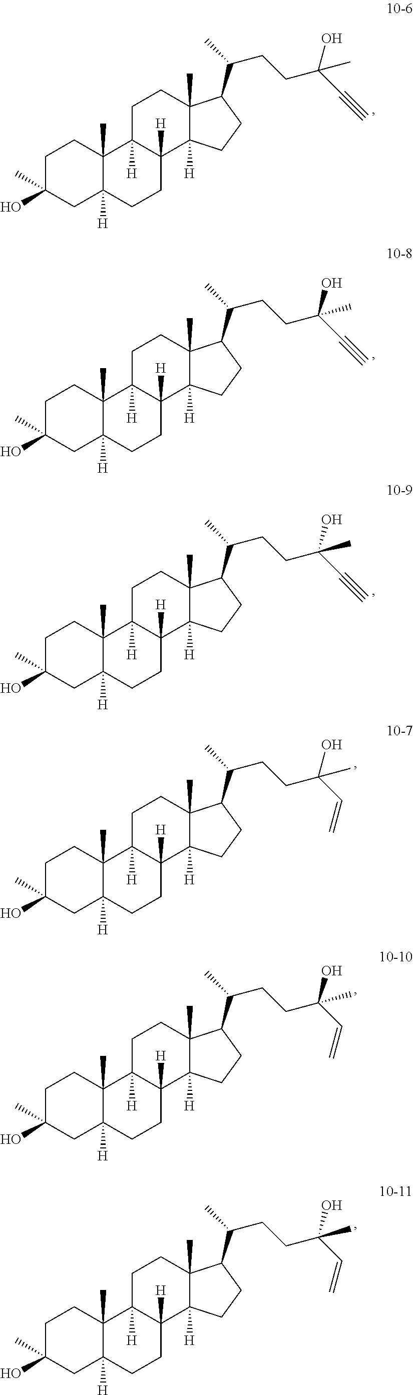

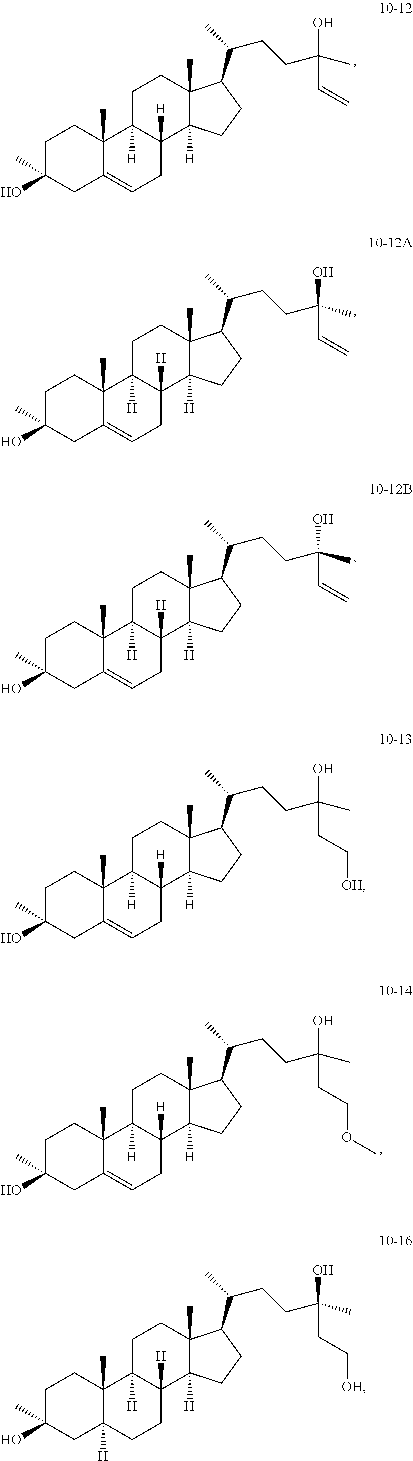

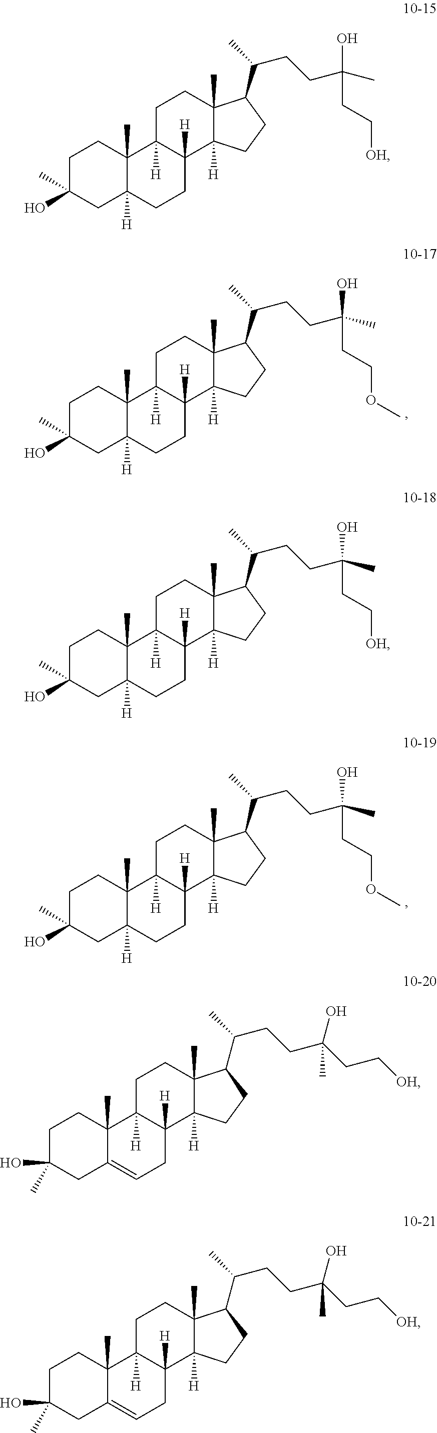

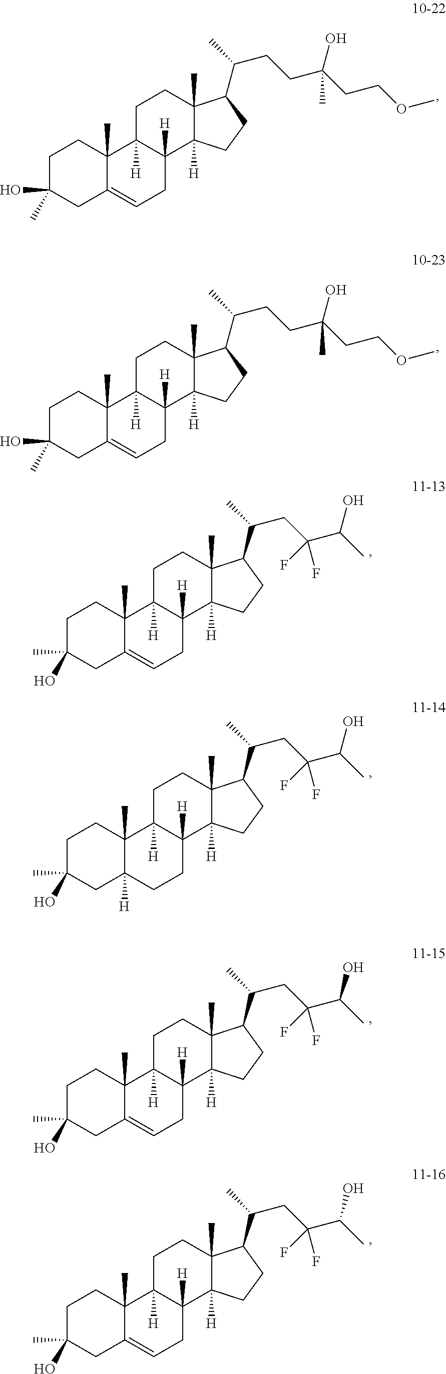

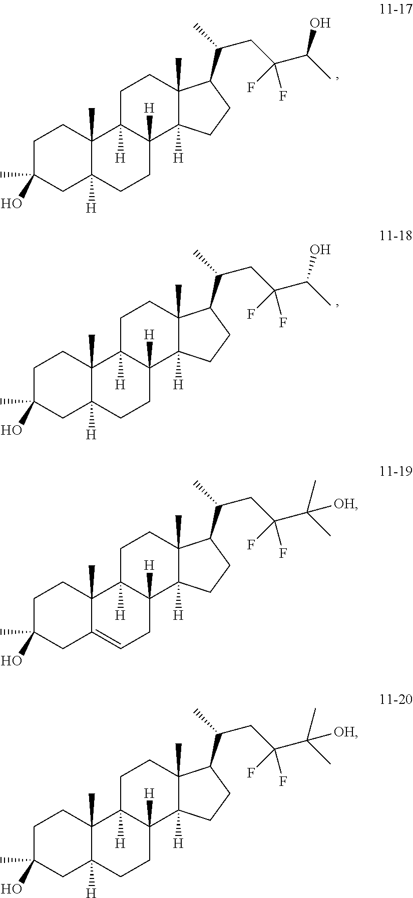

and pharmaceutically acceptable salts thereof.

Compounds of the Formula (I), and related compounds are described in WO2013/036835, WO2014/160480, and WO2014/160441, the contents of which are incorporated in their entirety.

Exemplary compounds of the invention also include compounds of the Formula (II-a):

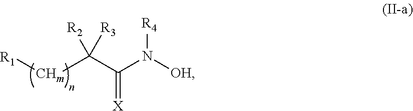

or a pharmaceutically acceptable salt thereof, where m may be an integer with a value ranging from zero to two; n may be an integer with a value ranging from one to six; R₂ and R₃ may include an amino group, a small alkyl, or a halide. One of either R₂ or R₃ may include an amino group and the other a small alkyl such as methyl, ethyl propyl, or halogen group such as fluoro, chloro and bromo. R₄ may include a hydrogen, small alkyl, substituted alkyl; X may include an oxygen or sulfur; R₁ and R₂ may include a hydrogen, alkyl, substituted alkyl, alkenyl, substituted alkenyl, alkynyl, substituted alkynyl, cycloalkyl, substituted cycloalkyl, cycloalkenyl, substituted cycloalkenyl, phenyl, substituted phenyl, heterocyclic, halide, nitrate, nitrite, nitrile, hydroxyl, thiol, sulfonamide, amine, guanidine, isoguanidine, cyanate, isocyanate, and carboxylate, or one of the following structural formulae:

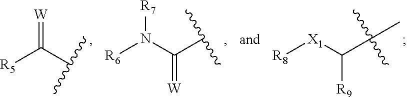

where X may be oxygen, sulfur, —S(O)— or —S(O)₂—, ═NH, ═NCN, X₁ is O, S, —S(O)— or —S(O)₂—;
W may be oxygen, sulfur, or pharmaceutically-acceptable salts thereof; R₅ may include an alkoxy, alkyl, substituted alkyl, alkenyl, substituted alkenyl, alkynyl, substituted alkynyl, cycloalkyl, substituted cycloalkyl, cycloalkenyl or substituted cycloalkenyl; R₆ and R₇ may include a hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R₆ and R₇ may be joined to form an alkylene or substituted alkylene group having from two to ten carbon atoms; R₈ may include an alkyl, substituted alkyl, alkenyl, substituted alkenyl, alkynyl, substituted alkynyl, cycloalkyl, substituted cycloalkyl, cycloalkenyl and substituted cycloalkenyl; and R₉ may include a hydrogen, alkyl, substituted alkyl, alkenyl, substituted alkenyl, alkynyl, substituted alkynyl, cycloalkyl, substituted cycloalkyl, cycloalkenyl and substituted cycloalkenyl; or R₈ and R₉ may be joined to form an alkylene or substituted alkylene group having from two to ten carbon atoms; or R₁ and R₂ may be selected from the group consisting of CH₃O, C₅H₉O—, C₆H₅SO₂O—, CH₃CO—, C₆H₅SO₂NH—, (C₆H₅SO₂)₂N—, C₄H₈N—, C₅H₁₀N—, and C₅H₁₁NN—.

Exemplary compounds of the invention also include compounds of the Formula (II-b):

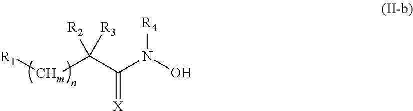

or a pharmaceutically acceptable salt thereof, wherein:
R₁ is selected from the group consisting of C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆substituted cycloalkyl, phenyl, cyano, hydroxyl, thiol, sulfonamide, amine, or:

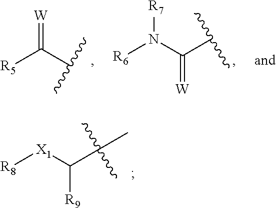

X is oxygen or sulfur;
X₁ is O, S, —S(O)— or —S(O)₂, —;
W is oxygen or sulfur;
R₅ is selected from the group consisting of alkoxy, alkyl, substituted alkyl, alkenyl, substituted alkenyl, alkynyl, substituted alkynyl, cycloalkyl, substituted cycloalkyl, cycloalkenyl and substituted cycloalkenyl;
R₆ and R₇ are each independently selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆6substituted cycloalkyl; or R₆ and R₇ are joined to form an C₃₋₁₀-cycloalkyl;
R₈ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆6substituted cycloalkyl; and
R₉ is selected from the group consisting of hydrogen, C₁₋₆alkyl, C₁₋₆substituted alkyl, C₂₋₆alkenyl, C₂₋₆substituted alkenyl, C₂₋₆alkynyl, C₂₋₆substituted alkynyl, C₃₋₆cycloalkyl, C₃₋₆6 substituted cycloalkyl;
R₂ is selected from the group consisting of hydrogen and C₁₋₆ alkyl;
R₃ is selected from the group consisting of C₁₋₆alkyl-NH—, NH₂—, -alkyl-C(O)—NH—, C₆H₅SO₂NH—, (C₆H₅SO₂)₂N—, C₄H₈N—, and C₅H₁₁NN—;
R₄ is selected from the group consisting of hydrogen, C₁₋₆alkyl, C₁₋₆substituted alkyl.

In some embodiments, R₂ may be hydrogen. In another embodiment, R₄ may be H, or R₄ may be a lower alkyl group, e.g., methyl, ethyl, propyl, isobutyl, t-butyl, n-butyl, isopropyl, etc.

In certain embodiments, X is oxygen. In another embodiment, R₃ may be NH₂ or CH₃—C(O)—NH—.

R₁ may be an alkyl group, e.g. a straight or branched alkyl, such as iso-butyl, propyl, ethyl, methyl, t-butyl, n-butyl, etc. In some embodiments, R₂ and R₃ are connected to a chiral center.

In some embodiments, the 3,4,5,-trisubstituted aryl amino hydroxamic acid may include one or more chiral centers. Such compounds may be prepared as a racemic mixture. If desired, however, such compounds may be prepared or isolated as pure stereoisomers, i.e., as individual enantiomers or diastereomers, or as stereoisomer-enriched mixtures. All such stereoisomers and enriched mixtures of the alkyl amino hydroxamic acid of Formula (II-a) and (II-b) are included within the scope of the present disclosure. Pure stereoisomers or enriched mixtures may be prepared using, for example, optically active starting materials or stereoselective reagents well known in the art. Alternatively, racemic mixtures of such compounds may be separated using, for example, chiral column chromatography, chiral resolving agents and the like.

In some embodiments, the compound is selected from:

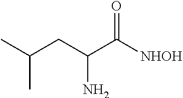

(AK-10), or 2-amino-N-hydroxy-4-methylpentamide (Salt TFA);

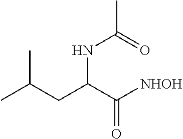

(AK-12), or 2-acetoamido-N-hydroxy-4-methylpentamide;

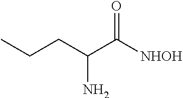

(AK-25), or 2-amino-N-hydroxypentamide (Salt TFA);

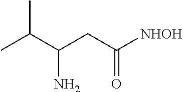

(AK-26),or 3-amino-N-hydroxy-4-methylpentamide (Salt TFA);

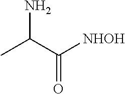

(AK-27), or 2-amino-N-hydroxypropanamide (Salt TFA);

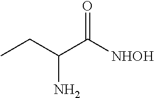

(AK-28), or 2-amino-N-hydroxybutanamide (Salt TFA);

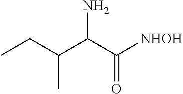

(AK-29), or 2-amino-N-hydroxy-3-methylpentamide (Salt TFA);

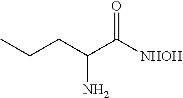

(AK-30), or 2-amino-N-hydroxy-4-methylpentamide (Salt TFA), and pharmaceutically acceptable salts thereof.

Compounds of the Formula (II-a) and (II-b), and related compounds are described in US20140045943, the contents of which are incorporated in its entirety.

Exemplary compounds of the invention also include a compound of the Formula (III):

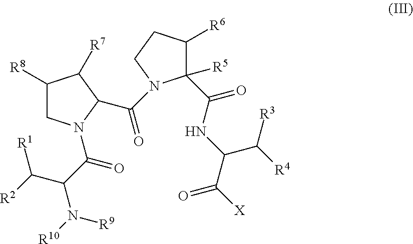

and pharmaceutically acceptable salts, stereoisomers, metabolites, and hydrates thereof, wherein:
R¹, R², R³, and R⁴ may be independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein each occurrence of R^{x} is independently selected from the group consisting of hydrogen; halogen; acyl; optionally substituted aliphatic; optionally substituted heteroaliphatic; optionally substituted aryl; and optionally substituted heteroaryl;
R⁵ and R⁶ may be independently selected from the group consisting of -Q-Ar and hydrogen, provided that at least one of R⁵ and R⁶ is -Q-Ar; wherein Q is independently selected from the group consisting of cyclic or acyclic, substituted or unsubstituted, branched or unbranched aliphatic; cyclic or acyclic, substituted or unsubstituted, branched or unbranched heteroaliphatic; and a bond; and wherein Ar is selected from the group consisting substituted or unsubstituted aryl, and substituted or unsubstituted heteroaryl; or R⁵ and R⁶, together with the atoms to which they are attached, form a substituted or unsubstituted 4-6 membered heterocyclic or cycloalkyl ring;
R⁷ and R⁸ may be independently selected from the group consisting of hydrogen; halogen; hydroxyl; substituted or unsubstituted C₁-C₆ alkyl; substituted or unsubstituted C₁-C₆ alkoxy; and substituted or unsubstituted aryl; or R⁷ and R⁸, together with the atoms to which they are attached, form a substituted or unsubstituted 4-6 membered heterocyclic or cycloalkyl ring;
R⁹ and R¹⁰ may be independently selected from the group consisting of hydrogen; C₁-C₆ alkyl, optionally substituted by one or more substituents each independently selected from the group consisting of halogen, oxo, and hydroxyl; C₂₋₆alkenyl, optionally substituted by one or more substituents each independently selected from the group consisting of halogen, oxo, and hydroxyl; C₂₋₆alkynyl, optionally substituted by one or more substituents each independently selected from the group consisting of halogen, oxo, and hydroxyl; C₃₋₆cycloalkyl, optionally substituted by one or more substituents each independently selected from the group consisting of C₁₋₆alkyl, halogen, oxo, and hydroxyl; phenyl, optionally substituted by one or more substituents each independently selected from the group consisting of C₁₋₆alkyl; C₁₋₆alkoxy; halogen; hydroxyl; —C(O)R^{x}; —CO₂(R^{x}); —C(O)N(R^{x})₂; —C(NR^{x})N(R^{x})₂; and —C(R^{x})₃;
X is selected from the group consisting of OR^{x} or NR^{x}R^{x}; wherein each occurrence of R^{x} is independently selected from the group consisting of hydrogen; halogen; C₁₋₆alkyl; C₂₋₆alkenyl; C₂₋₆alkynyl; C₃₋₆cycloalkyl; and phenyl; or R⁹ and R¹⁰, together with N, form a 4-6 membered heterocyclic ring, optionally substituted by one or more substituents each independently selected from the group consisting of C₁₋₆alkyl, halogen, oxo, and hydroxyl.

In some embodiments, the compound of the Formula (III) is a compound of the Formula (III-A) (III-B), and (III-C):

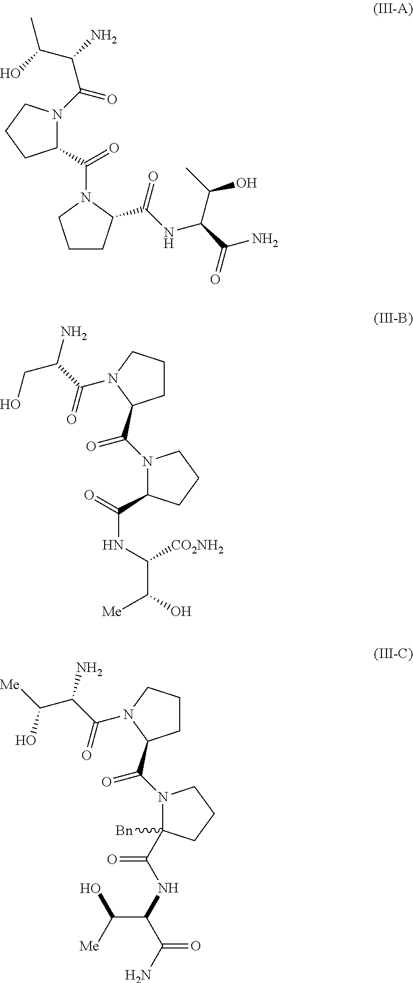

The compound of the Formula (III-A) is also referred to as Glyx-13. Compounds of the Formula (III) are described in U.S. Pat. No. 8,673,843, the contents of which are incorporated in its entirety.

Exemplary compounds of the present invention also include compounds of the Formula (IV):

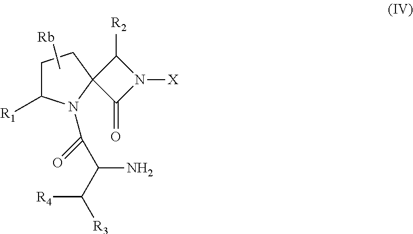

and pharmaceutically acceptable salts, stereoisomers, and N-oxides thereof, wherein
Rb is selected from the group consisting of H, halogen, hydroxyl, cyano and C₁-C₆ alkyl;
R₁ is H or C₁-C₆ alkyl;
R₂ is H or C₁-C₆ alkyl;
R₃ is selected from the group consisting of H, C₁-C₆ alkyl, —OH, C₁-C₆ alkoxy, —OC(O)—C₁-C₆ alkyl and —OC(O)-phenyl (optionally substituted by one, two or three substituents selected from the group consisting of halogen, hydroxyl, C₁-C₆ alkyl, and C₁-C₆ alkoxy);
R₄ is H or C₁-C₆ alkyl; and
X is selected from the group consisting of hydrogen, —C₁-C₆ alkylene-C₁-C₃ cycloalkyl; C₁-C₆ alkylene-heterocycle (optionally substituted by one, two or three substituents selected from the group consisting of halogen, hydroxyl, C₁-C₆ alkyl, and C₁-C₆ alkoxy), and —C₁-C₆ alkylene-heteroaryl (optionally substituted by one, two or three substituents selected from the group consisting of halogen, hydroxyl, C₁-C₆ alkyl, and C₁-C₆ alkoxy);
   or in other embodiments, the variables set forth in formula (III) are as defined as follows:
Rb is selected from the group consisting of H, halogen, hydroxyl, cyano and C₁-C₆ alkyl (e.g., H);
R₁ is H or C₁-C₆ alkyl;
R₂ is H or C₁-C₆ alkyl;
R₃ is selected from the group consisting of H, C₁-C₆ alkyl, —OH, C₁-C₆ alkoxy, —OC(O)—C₁-C₆ alkyl and —OC(O)-phenyl (optionally substituted by one, two or three substituents independently selected from the group consisting of halogen, hydroxyl, C₁-C₆ alkyl, and C₁-C₆ alkoxy);
R₄ is H or C₁-C₆ alkyl;
X is selected from the group consisting of:
   (i) hydrogen;
   (ii) —C₁-C₆ alkylene-C₃-C₆ cycloalkyl;
   (iii) —C₁-C₆ alkylene-heterocyclyl including from 3 to 6 ring atoms wherein 1, 2, or 3 of the ring atoms are independently selected from the group consisting of N, NH, (C₁-C₃ alkyl), O, and S; wherein the heterocyclyl is optionally substituted by one, two or three substituents independently selected from the group consisting of halogen, hydroxyl, C₁-C₆ alkyl, and C₁-C₆ alkoxy);
   (iv) —C₁-C₆ alkylene-C(O)-heterocyclyl including from 3 to 6 ring atoms wherein 1, 2, or 3 of the ring atoms are independently selected from the group consisting of N, NH, N(C₁-C₃ alkyl), O, and S; wherein the heterocyclyl is optionally substituted by one, two or three substituents independently selected from the group consisting of halogen, hydroxyl, C₁-C₆ alkyl, and C₁-C₆ alkoxy);
   (v) —C₁-C₆ alkylene-heteroaryl including from 5 to 6 ring atoms wherein 1, 2, or 3 of the ring atoms are independently selected from the group consisting of N, NH, N(C₁-C₃ alkyl), O, and S; wherein the heteroaryl is optionally substituted by one, two or three substituents independently selected from the group consisting of halogen, hydroxyl, C₁-C₆ alkyl, and C₁-C₆ alkoxy;
   (vi) branched unsubstituted C₃-C₆ alkyl; and
   (vii) branched C₃-C₆ alkyl substituted with —C(O)NH2 on one carbon and —OH on another carbon;
and wherein the −N¾ group attached to the carbon adjacent to —CH(R₃)(R₄) is optionally substituted with a substituent selected from —C(O)OR₃₁ and —C(O) R₃₂, wherein:
R₃₁ is selected from the group consisting of: C₁-C₆ alkyl; C₁-C₆ haloalkyl; C₂-C₆ alkenyl; C₂-C₆ alkynyl; C₃-C₁₀ cycloalkyl, wherein the C₃-C₁₀ cycloalkyl is optionally substituted with from 1-3 independently selected C₁-C₃ alkyl; —CH₂-C₃-C₁₀ cycloalkyl wherein the C₃-C₁₀ cycloalkyl is optionally substituted with from 1-3 independently selected C₁-C₃ alkyl; —CH2-phenyl, wherein the phenyl is optionally substituted with from 1-2 substituents independently selected from C₁-C₃alkyl; C₁-C₃ haloalkyl; C₁-C₃ alkoxy; C₁-C₃ haloalkoxy; nitro; halo; SO₂Me, cyano; and —OC(O)CH₃; and —CH₂— pyridyl; and
R₃₂ is selected from the group consisting of: H; C₁-C₆ alkyl; C₁-C₆ haloalkyl; phenyl, wherein the phenyl is optionally substituted with from 1-2 substituents independently selected from C₁-C₃ alkyl; C₁-C₃ haloalkyl; C₁-C₃ alkoxy; C₁-C₃ haloalkoxy; nitro; halo; SO₂Me, cyano; and —OC(O)CH₃; and pyridyl.

In some embodiments, the compound of the Formula (IV) is a compound of the formula:

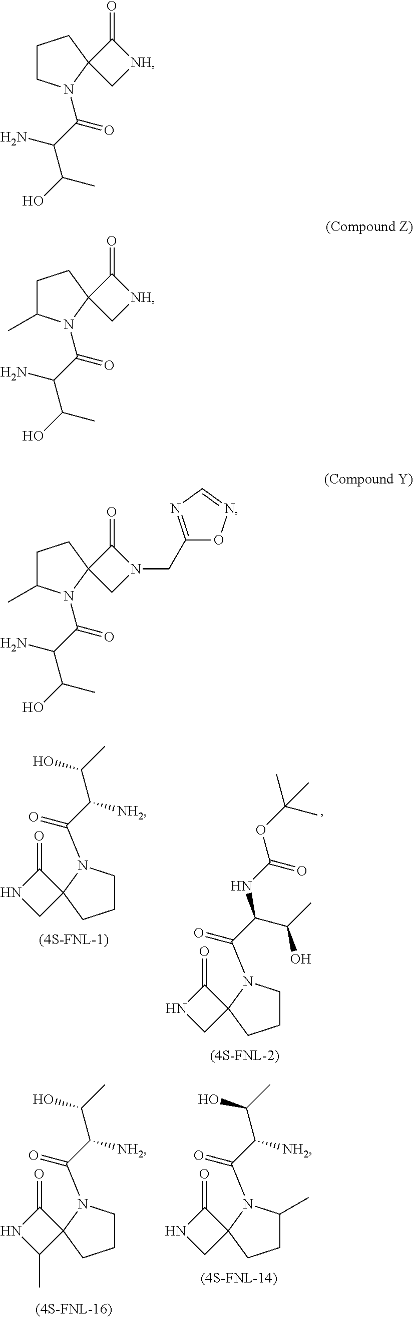

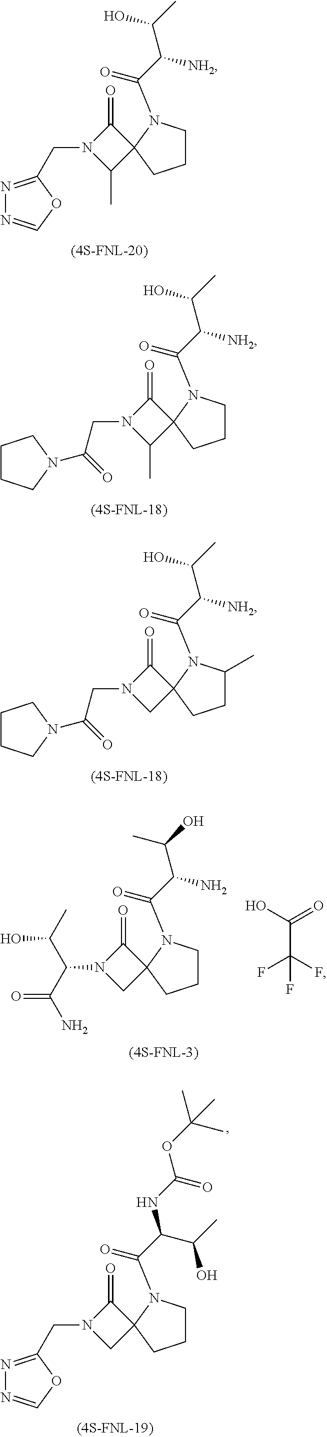 [SMILES: CC1N(Cc2nnco2)C(=O)C12CCCN2C(=O)[C@@H](N)[C@@H](C)O ; CC1N(CC(=O)N2CCCC2)C(=O)C12CCCN2C(=O)[C@@H](N)[C@@H](C)O ; CC1CCC2(CN(CC(=O)N3CCCC3)C2=O)N1C(=O)[C@@H](N)[C@@H](C)O ; C[C@@H](O)[C@H](N)C(=O)N1CCCC12CN([C@H](C(N)=O)[C@@H](C)O)C2=O ; O=C(O)C(F)(F)F ; C[C@@H](O)[C@H](NC(=O)OC(C)(C)C)C(=O)N1CCCC12CN(Cc1nnco1)C2=O]

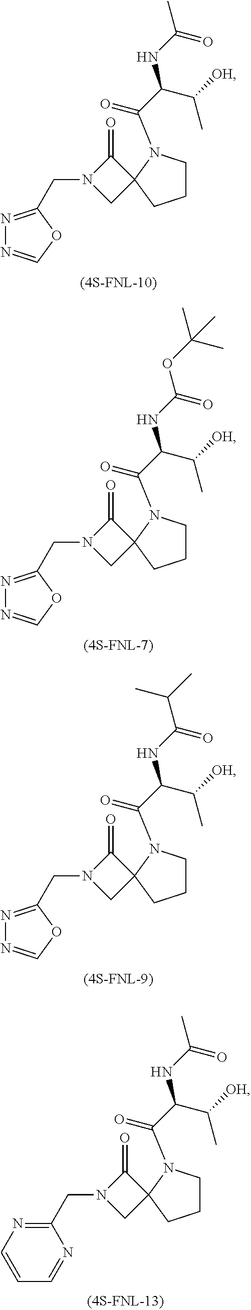 [SMILES: CC(=O)N[C@H](C(=O)N1CCCC12CN(Cc1nnco1)C2=O)[C@@H](C)O ; C[C@@H](O)[C@H](NC(=O)OC(C)(C)C)C(=O)N1CCCC12CN(Cc1nnco1)C2=O ; CC(C)C(=O)N[C@H](C(=O)N1CCCC12CN(Cc1nnco1)C2=O)[C@@H](C)O ; CC(=O)N[C@H](C(=O)N1CCCC12CN(Cc1ncccn1)C2=O)[C@@H](C)O]

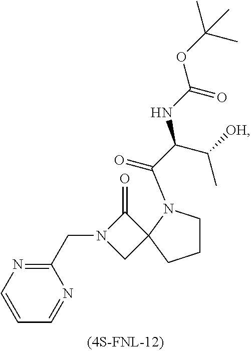 [SMILES: C[C@@H](O)[C@H](NC(=O)OC(C)(C)C)C(=O)N1CCCC12CN(Cc1ncccn1)C2=O]

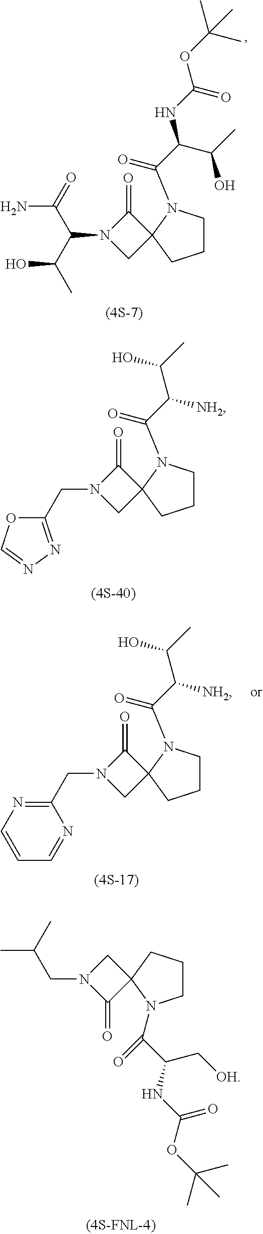

Compounds of the Formula (IV) are described in WO2014/120786, the contents of which are incorporated in its entirety.

Exemplary compounds of the present invention also include a compound of the Formula (V):

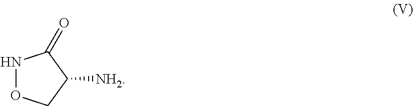

The compound of the Formula (V) is also referred to as 4-amino-3-isoxazolidinone, (R)-4-amino-1,2-oxazolidin-3-one, cycloserine, and seromycin.

Exemplary compounds of the present invention also include a compound of the Formula (VI):

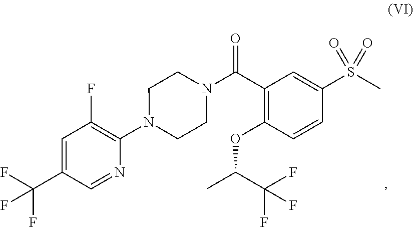 [SMILES: C[C@H](Oc1ccc(S(C)(=O)=O)cc1C(=O)N1CCN(c2ncc(C(F)(F)F)cc2F)CC1)C(F)(F)F]

or a pharmaceutically acceptable salt thereof.

The compound of the Formula (VI) is also referred to as [4-[3-fluoro (trifluoromethyl)pyridin-2-yl]piperazin-1-yl}(5-methylsulfonyl)-2-[(1S)-2,2,2-trifluoro-1-methylethoxy]phenyl}methanone, RG1678, RO-4917838, and bitopertin. In some embodiments, the compounds of the invention (e.g., the compound of Formula (VI)) are glycine reuptake inhibitors. In some embodiments, the compounds of the invention (e.g., the compound of Formula (VI)) are glycine transporter 1 (GlyT1) inhibitors. In some embodiments, the compounds of the invention (e.g, GlyT1) inhibitors are described in U.S. Pat. No. 8,524,909 and US 20130158050, the contents of which are incorporated in its entirety.

In some embodiments, the compounds of the invention (e.g., the compound of Formula (VI)) are glycine reuptake inhibitors.

Exemplary compounds of the present invention also include compounds of the Formula (VII):

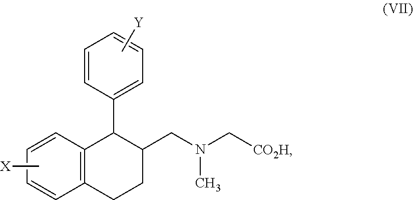

wherein:
X is 1-3 substituents selected from hydrogen, halogen, methyl, methoxy, trifluoromethyl, and trifluoromethoxy; and
Y is 1-3 substituents selected from hydrogen, methyl, and halogen; or a pharmaceutically acceptable salt thereof.

In some embodiments, the compounds (e.g., compounds of the Formula (VII)) are glycine transporter-1 inhibitors. In some embodiments, the compound of the Formula (VII) is 2-([(1R,2S)-6-methoxy-1-phenyl-1,2,3,4-tetrahydronaphthalen-2-yl]methyl-methylamino)acetic acid. In some embodiments, the compound is Org 25935. In some embodiments, the compound of the Formula (VII) is a compound of the Formula (VII-a):

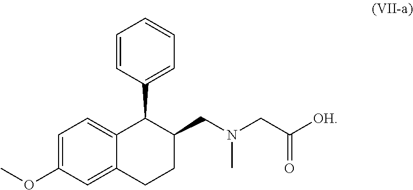 [SMILES: COc1ccc2c(c1)CC[C@H](CN(C)CC(=O)O)[C@@H]2c1ccccc1]

Compounds of the Formula (VII) are described in WO2009/059961, the contents of which are incorporated in its entirety.

Exemplary compounds of the present invention also include a compound of the Formula (VIII):

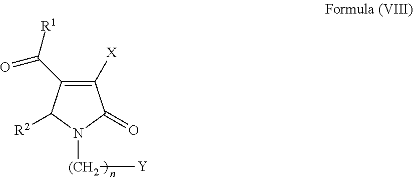

or salts thereof, wherein
X is OH or NH₂, wherein X optionally substituted with J;
Y is a bicyclic carbocyclyl or Ar¹ is aryl, heterocyclyl, bicyclic heterocyclyl, bicyclic heterocycle comprising one five-membered ring and one six-membered ring, bicyclic heterocycle comprising one five-membered heterocyclic ring and one six-membered aryl ring, bicyclic heterocycle comprising one five-membered heterocyclic ring and one six-membered heterocyclic ring, a bicyclic heterocycle comprising two six-membered rings; a bicyclic heterocycle comprising two six-membered aryl rings, a bicyclic heterocycle comprising two six-membered heterocyclic rings, a bicyclic heterocycle comprising one heterocyclic six-membered ring and one aromatic six-membered ring, or an bicyclic aryl, wherein Y or Ar¹ is optionally substituted with one or more, the same or different, J;
n is 0, 1, 2, 3, 4, or 5;
R¹ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfamoyl, alkylsulfonyl, arylsulfonyl, carbocyclyl, aryl, or heterocyclyl, wherein R¹ is optionally substituted with one or more, the same or different, J;
R is hydrogen, alkyl, halogen, nitro, cyano, hydroxy, amino, mercapto, formyl, carboxy, alkanoyl, carbamoyl, alkoxy, alkylthio, alkylamino, (alkyl)₂amino,
alkylsulfamoyl, alkylsulfonyl, arylsulfonyl, carbocyclyl, aryl, or heterocyclyl, wherein R is optionally substituted with one or more, the same or different, J; and
J is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfamoyl,
alkylsulfonyl, arylsulfonyl, carbocyclyl, aryl, or heterocyclyl, wherein J is optionally substituted with one or more, the same or different, K;
K is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfamyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In some embodiments, the compound of the Formula (VIII) is of the formula:

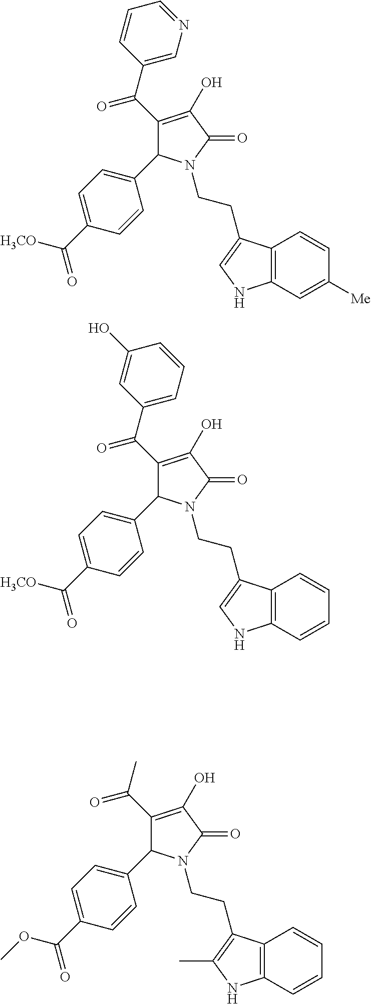 [SMILES: COC(=O)c1ccc(C2C(C(=O)c3cccnc3)=C(O)C(=O)N2CCc2c[nH]c3cc(C)ccc23)cc1 ; COC(=O)c1ccc(C2C(C(=O)c3cccc(O)c3)=C(O)C(=O)N2CCc2c[nH]c3ccccc23)cc1 ; COC(=O)c1ccc(C2C(C(C)=O)=C(O)C(=O)N2CCc2c(C)[nH]c3ccccc23)cc1]

Compounds of the Formula (VIII) are described in WO2014/025942, the contents of which are incorporated in its entirety.

Exemplary compounds of the present invention also include a compound of the Formulas (IX-A) and (IX-B) provided below:

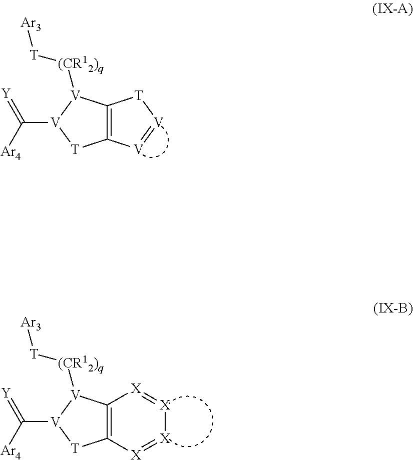

wherein for Formulas (IX-A) and (IX-B),

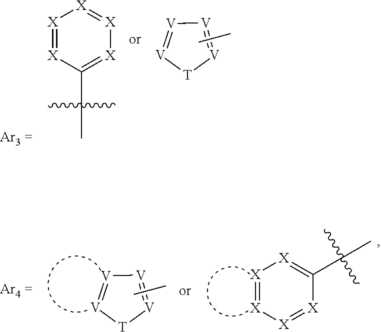

X is, independently, N or C bonded to H or a substituent, J, with the proviso that no more than three of X are N;
Y is independently selected from O, S, NR′, CH₂, and CR¹₂;
R¹ and R² are, independently, selected from H, alkyl, substituted alkyl, alkenyl, substituted alkenyl, aryl, substituted aryl, heteroaryl, substituted heteroaryl, and hydroxy, and, when R′ is attached to a carbon atom, it can be halo or cyano,
T is, independently, CHR¹, CR¹₂, O, S, or NR¹,
V is, independently, N, or C bonded to H or a substituent J,
J is a non-hydrogen substituent selected from the group consisting of halo (—F, —Cl, —Br, —I), nitro, amino (NR¹R²), OR¹, SR¹, —R¹, —CF₃, —CN, —C₂R¹, —SO₂CH₃, —C(═O)NR¹R²—NR, C(═O)R¹, —C(═O)R¹, —C(═O)OR¹, —(CH₂)_{q}OR¹, —OC(═O)R¹, —OC(═O)NR¹R², —NR¹(C═Y)—NR¹R², —NR¹(C═Y)—OH, —NR¹(C═Y)—SH, sulfonyl, sulfinyl, phosphoryl, and azo, and q is 0-5.

In some embodiments, the compound of the Formula (IX-A) and (IX-B) is of the formula:

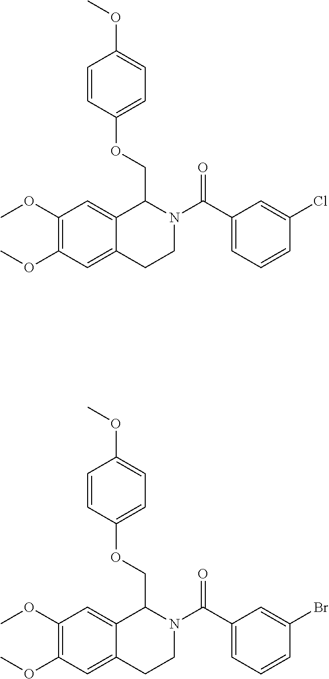 [SMILES: COc1ccc(OCC2c3cc(OC)c(OC)cc3CCN2C(=O)c2cccc(Cl)c2)cc1 ; COc1ccc(OCC2c3cc(OC)c(OC)cc3CCN2C(=O)c2cccc(Br)c2)cc1]

Compounds of the Formula (IX-A) and (IX-B) are described in WO2010/088414 and US 2014/0275529, the contents of which are incorporated in its entirety.

Exemplary compounds of the present invention also include a compound of the Formula (X):

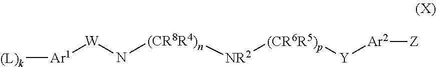

wherein:
each L is independently C₁-C₆ alkyl, C₁-C₆alkoxy, C(═O)—(C₁-C₆)-alkyl, C₁-C₆haloalkyl, alkaryl, hydroxy, —O-alkyl, —O-aryl, —SH, —S-alkyl, —S-aryl, fluoro, chloro, bromo, iodo, nitro, or cyano; or two L groups may be taken together with Ar¹ to form: a dioxolane ring or a cyclobutane ring;
k=0, 1, 2, 3, 4 or 5;
each Ar¹ and Ar² is independently aryl or heteroaryl;
W is a bond, C₁-C₄ alkyl, or C₂-C₄ alkenyl;
X is a bond, NR¹ or O;
each R¹ and R² is independently H, C₁-C₆ alkyl, C₂-C₆ alkenyl or C₆-C₁₂ aralkyl; or
   R¹ and R² can be taken together to form a 5-8 membered ring;
   each R³ and R⁴ is independently H, C₁-C₆ alkyl, C₁-C₆alkoxy, C(═O)—(C₁-C₆)-alkyl, C₁-C₆ haloalkyl, hydroxy, fluoro, chloro, bromo, iodo, nitro, or cyano; or CR³R⁴ is C═O;
   n and p are each independently 1, 2, 3 or 4;
   each R⁵ and R⁶ is independently H, C₁-C₆ alkyl, C₁-C₆alkoxy, C(═O)—(C₁-C₆)-alkyl, C₁-C₆ haloalkyl, hydroxy, fluoro, chloro, bromo, iodo, nitro, or cyano; or CR⁵R⁶ is C═O or CH₂;
   or wherein —NR²—(CR⁵R⁶)ₚ— can be

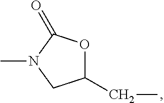

Y is a bond, O, S, SO, SO₂, CH₂, NH, N(C₁-C₆ alkyl), or NHC(═O);
Z is OH, NR⁶R⁷, NR⁸SO₂(C₁-C₆ alkyl), NR⁸C(O)NR⁶R⁷, NR⁸C(S)NR⁶R⁷, NR⁸C(O)O(C₁-C₆ alkyl),
NR⁸-dihydrothiazole, or NR⁸-dihydroimidazole; wherein each R⁶, R⁷ and R⁸ is independently H, C₁-C₆ alkyl or C₆-C₁₂ aralkyl; or

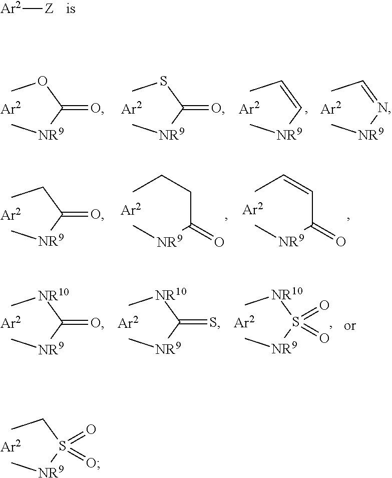

wherein R⁹ and R¹⁰ are each independently H, C₁-C₆ alkyl, aralkyl.

Compounds of the Formula (X) are described in US 2011/0160223 and US 2014/0031363, the contents of which are incorporated in its entirety.

Exemplary compounds of the present invention also include a compound of the Formula (XI-A) or (XI-B):

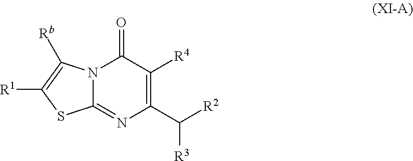

wherein
R^{a} is Ci-₆alkyl or C₂₋₆alkenyl, each optionally substituted with one or more R^{b} substituents; C₂-₆alkynyl; halo; —C(0)R^{c}; —NR^{d}R^{e}; —C(O)NR^{d}R^{e}; —C(S)NR^{d}R^{e}; —C(═N—OH)—C₁₋₄alkyl; —OC₁₋₄alkyl; —OC₁₋₄haloalkyl; —SC₁₋₄alkyl; —S0₂C₁₋₄alkyl; cyano; C₃₋₆cycloalkyl optionally substituted with one or more R^{f} substituents; or a phenyl, monocyclic heteroaryl, or heterocycloalkyl ring, each ring optionally substituted with one or more R^{g} substituents;
   wherein each R^{b} substituent is independently selected from the group consisting of —OH, —Ci_₄alkoxy, —NR^{d}R^{e}, —C(O)NR^{d}R^{e}, —SC₁₋₄alkyl, —SO₂C₁₋₄alkyl, cyano, halo, C₃₋₆ cycloalkyl, and monocyclic heteroaryl;
R^{c} is C₁₋₄alkyl, —C₁₋₄haloalkyl, C₃₋₆cycloalkyl, or a monocyclic, carbon-linked heterocycloalkyl;
R^{d} is H or C₁₋₄alkyl;
R^{e} is H; C₁₋₄alkyl optionally substituted with —CN, —CF₃, —OH, or a monocyclic heterocycloalkyl; C₃₋₆cycloalkyl; —OH; or —OC₁₋₄alkoxy;
or R^{d} and R^{e} taken together with the nitrogen to which they are attached form a heterocycloalkyl, optionally substituted with C₁₋₄alkyl or —OH;
each R^{f} substituent is independently selected from the group consisting of: C₁₋₄alkyl optionally substituted with —OH, cyano, or C₁₋₄alkoxy; —OH; halo; C₁₋₄haloalkyl; —CONH₂; and cyano; and each R^{g} substituent is independently selected from the group consisting of C₁₋₄alkyl, —CF₃, halo, —NH₂, —OCH₃, cyano, and —OH;
R¹ is selected from the group consisting of H, C₁₋₆alkyl,
   C₁₋₄haloalkyl, C₃₋₆cycloalkyl, halo, —OC₁₋₄alkyl, cyano, and —C(O)C₁₋₄alkyl; or R^{a} and R¹ taken together with the carbons to which they are attached form a 5- to 7-membered ring, optionally containing an O or NH, and optionally substituted with one or more R^{h} substituents;
   wherein each R^{h} substituent is independently —C(O)NR¾^{j}, cyano, or is C₁₋₄alkyl optionally substituted with —OH, —OCH₃, cyano, or —C(O)NR¾^{j}; or two R^{h} groups attached to the same carbon and taken together with the carbon to which they are attached form a carbonyl or a C₃₋₆cycloalkyl;
   wherein R¹ and R^{J} are each independently H or C₁₋₄alkyl;
R² is —R^{m}, —OR^{m}, or —NR^{m}Rⁿ;
   wherein R^{m} is aryl or heteroaryl, each optionally substituted with one or more R^{s} substituents;
   wherein each R^{s} substituent is independently selected from the group consisting of C₁₋₄alkyl, C₂₋₄alkenyl (optionally substituted with halo), C₂₋₄alkynyl, C₁₋₄haloalkyl, C₁₋₄alkoxy, C₁₋₄alkyl-OH, C₁₋₄haloalkoxy, halo, cyano, C₃₋₆cycloalkyl (optionally substituted with —OH or halo), monocyclic heteroaryl, —NH₂, —NO₂, —NHS0₂C₁₋₄alkyl, and —SO₂C₁₋₄alkyl;
Rⁿ is H, C₁₋₄haloalkyl, or C₁₋₄alkyl optionally substituted with —OH or C₁₋₄alkoxy;
or R^{m} and R¹⁰ taken together with the nitrogen to which they are attached form a pyrrolidine or piperidine ring, optionally substituted with C₁₋₄alkyl and optionally fused to phenyl, wherein said phenyl is optionally substituted with halo;
R³ is H or methyl; and
R⁴ is H or fluoro; or a pharmaceutically acceptable salt thereof.
   In one aspect, the invention is directed to a compound of Formula (XI-B):

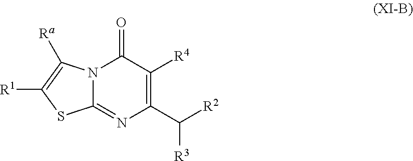

wherein
R^{a} is Ci-₆alkyl optionally substituted with one or more R^{b} substituents; C₂₋₆alkenyl; C₂₋₆alkynyl; halo; —C(O)R^{c}; —NR^{d}R^{e}; —C(O)NR^{d}R^{e}; —C(S)NR^{d}R^{e}; —C(═N—OH)—C₁₋₄alkyl; —SO₂C₁₋₄alkyl; cyano; C₃₋₆cycloalkyl optionally substituted with one or more R^{f} substituents; or a phenyl, monocyclic heteroaryl, or heterocycloalkyl ring, each ring optionally substituted with one or more R^{g} substituents;
   wherein each R^{b} substituent is independently selected from the group consisting of —OH, —C₁₋₄alkoxy, —NR^{d}R^{e}, —C(O)NR^{d}R^{e}, —SC₁₋₄alkyl, —SO₂C₁₋₄alkyl, cyano, halo, and monocyclic heteroaryl;
R^{c} is C₁₋₄alkyl, —C₁₋₄haloalkyl, C₃₋₆cycloalkyl, or a monocyclic, carbon-linked heterocycloalkyl;
R^{d} is H or C₁₋₄alkyl;
R^{e} is H; C₁₋₄alkyl optionally substituted with —CN, —CF₃, —OH, or a monocyclic heterocycloalkyl; C₃₋₆cycloalkyl; —OH; or —OC₁₋₄alkoxy;
or R^{d} and R^{e} taken together with the nitrogen to which they are attached form a heterocycloalkyl, optionally substituted with C₁₋₄alkyl or —OH;
each R^{f} substituent is independently selected from the group consisting of: C₁₋₄alkyl optionally substituted with —OH, cyano, or C₁₋₄alkoxy; C₁₋₄haloalkyl; —CONH₂; and cyano; and
each R^{g} substituent is independently selected from the group consisting of C₁₋₄alkyl, —CF₃, halo, —NH₂, —OCH₃, cyano, and —OH;
R¹ is selected from the group consisting of H, C₁₋₆alkyl,
   C₁₋₄haloalkyl, and C₃₋₆cycloalkyl; or R^{a} and R¹ taken together with the carbons to which they are attached form a 5- to 7-membered ring, optionally containing an O or NH, and optionally substituted with one or more R^{h} substituents; wherein each R^{h} substituent is independently —C(O)NR¾^{j}, cyano, or is C₁₋₄alkyl optionally substituted with —OH, —OCH₃, cyano, or —C(O)NR¾^{j}; or two R^{h} groups attached to the same carbon and taken together with the carbon to which they are attached form a carbonyl or a C₃₋₆cycloalkyl;
   wherein R¹ and R^{J} are each independently H or C₁₋₄alkyl; R² is —R^{m}, —OR^{m}, or —NR^{m}Rⁿ;
   wherein R^{m} is aryl or heteroaryl, each optionally substituted with one or more R^{s} substituents;
   wherein each R^{s} substituent is independently selected from the group consisting of C₁₋₄alkyl, Q.₄haloalkyl, C₁₋₄alkoxy, C₁₋₄alkyl-OH, C₁₋₄haloalkoxy, halo, cyano, C₃₋₆cycloalkyl, —NHS0₂C₁₋₄alkyl, and —S0₂C₁₋₄alkyl;
Rⁿ is H, C₁₋₄haloalkyl, or C₁₋₄alkyl optionally substituted with —OH or C₁₋₄alkoxy;
or R^{m} and Rⁿ taken together with the nitrogen to which they are attached form a pyrrolidine or piperidine ring, optionally substituted with C₁₋₄alkyl and optionally fused to phenyl, wherein said phenyl is optionally substituted with halo;
R³ is H or methyl; and
R⁴ is H or fluoro; or a pharmaceutically acceptable salt thereof.
   Compounds of the Formula (XI-A) and (XI-B) are described in WO 2015/052226, the contents of which are incorporated in its entirety.

Exemplary compounds of the present invention also include a compound selected from:

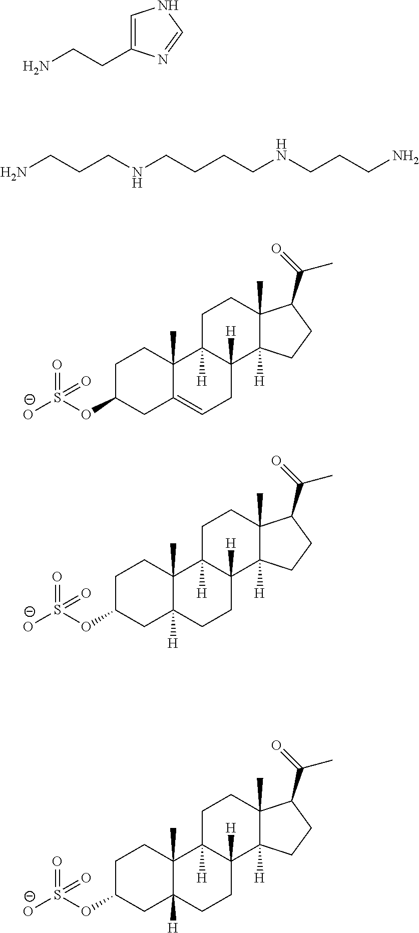

In some embodiments, the compound described herein is histamine, spermine, pregnenolone sulfate, allopregnanolone sulfate, or pregnanolone sulfate.

Exemplary compounds of the present invention also include a compound selected from:

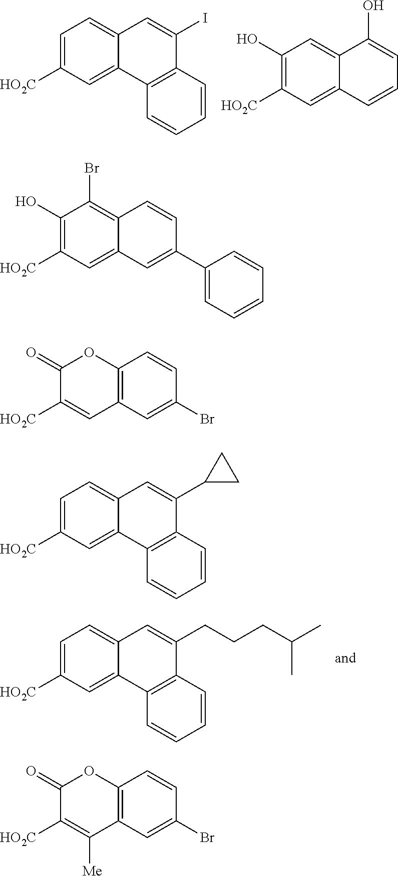

In some embodiments, the compounds of the present invention are described in Costa B M, Irvine M W, Fang, G, et al. A novel family of negative and positive allosteric modulators NMDA receptors. J Pharmacol Exp Ther 2010; 335(3):614-21, the contents of which are incorporated in its entirety.

In some embodiments, the compounds of the present invention are described in WO2015065891, WO2014120800, WO2014120789, WO2014120783, WO2014120786, WO2014120784, US20130035292, WO2011003064, WO2010033757, and WO2009039390, the contents of which are incorporated in its entirety.

Exemplary compounds of the present invention also include a compound selected from:

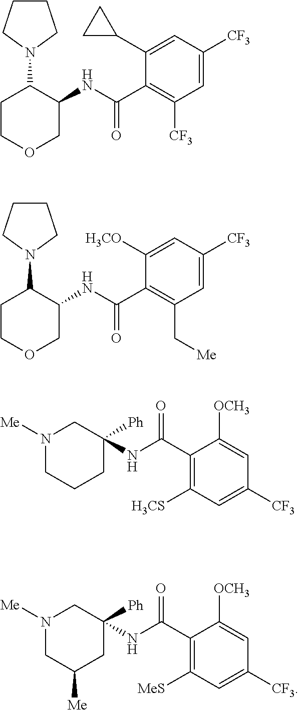

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Aliphatic” refers to an alkyl, alkenyl, alkynyl, or carbocyclyl group, as defined herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

As used herein, “alkylene,” “alkenylene,” and “alkynylene,” refer to a divalent radical of an alkyl, alkenyl, and alkynyl group, respectively. When a range or number of carbons is provided for a particular “alkylene,” “alkenylene,” and “alkynylene” group, it is understood that the range or number refers to the range or number of carbons in the linear carbon divalent chain. “Alkylene,” “alkenylene,” and “alkynylene” groups may be substituted or unsubstituted with one or more substituents as described herein.

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers to an alkenyl group wherein two hydrogens are removed to provide a divalent radical, and which may be substituted or unsubstituted. Exemplary unsubstituted divalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers to a linear alkynyl group wherein two hydrogens are removed to provide a divalent radical, and which may be substituted or unsubstituted. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and for 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and for 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and for 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “alkylene,” “alkenylene,” “alkynylene,” “heteroalkylene,” “heteroalkenylene,” and “heteroalkynylene,” refer to a divalent radical of an alkyl, alkenyl, alkynyl group, heteroalkyl, heteroalkenyl, and heteroalkynyl group respectively. When a range or number of carbons is provided for a particular “alkylene,” “alkenylene,” “alkynylene,” “heteroalkylene,” “heteroalkenylene,” or “heteroalkynylene,” group, it is understood that the range or number refers to the range or number of carbons in the linear carbon divalent chain. “Alkylene,” “alkenylene,” “alkynylene,” “heteroalkylene,” “heteroalkenylene,” and “heteroalkynylene” groups may be substituted or unsubstituted with one or more substituents as described herein.

“‘Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

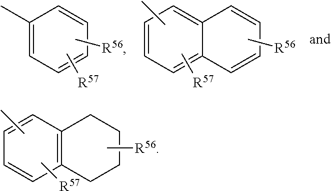

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹ NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

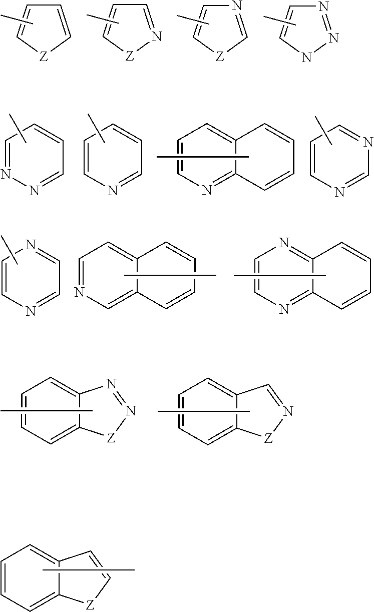

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Acylamino” refers to a radical —NR²²C(O)R²³, where each instance of R²² and R23 is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁-C₈ alkyl, —NR²⁴C(O)—(CH₂)ₜ(C₆-C₁₀ aryl), —NR²⁴C(O)—(CH₂)ᵣ(5-10 membered heteroaryl), —NR²⁴C(O)—(CH₂)ₜ(C₃-C₁₀ cycloalkyl), and —NR²⁴C(O)—(CH₂)ₜ(4-10 membered heterocyclyl), wherein t is an integer from 0 to 4, and each R²⁴ independently represents H or C₁-C₈ alkyl. In certain embodiments, R²⁵ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; and R²⁶ is H, C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxyl; provided at least one of R²⁵ and R²⁶ is other than H.

“Acyloxy” refers to a radical —OC(O)R²⁷, where R²⁷ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl. In certain embodiments, R²⁸ is C₁-C₈ alkyl, substituted with halo or hydroxy; C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R⁶² is not a hydrogen. In certain embodiments, R⁶² is selected from H, C₁-C₈ alkyl, C₃-C₁₀ cycloalkyl, 4-10 membered heterocyclyl, C₆-C₁₀ aryl, aralkyl, 5-10 membered heteroaryl, and heteroaralkyl; or C₁-C₈ alkyl substituted with halo or hydroxy; or C₃-C₁₀ cycloalkyl, 4-10 membered heterocyclyl, C₆-C₁₀ aryl, aralkyl, 5-10 membered heteroaryl, or heteroaralkyl, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; provided that at least one R⁶² is other than H.

Exemplary “substituted carbamoyl” groups include, but are not limited to, —C(O) NR⁶⁴—C₁-C₈ alkyl, —C(O)NR⁶⁴—(CH₂)ₜ(C₆-C₁₀ aryl), —C(O)N⁶⁴—(CH₂)ₜ(5-10 membered heteroaryl), —C(O)NR⁶⁴—(CH₂)ₜ(C₃-C₁₀ cycloalkyl), and —C(O)NR⁶⁴—(CH₂)ₜ(4-10 membered heterocyclyl), wherein t is an integer from 0 to 4, each R⁶⁴ independently represents H or C₁-C₈ alkyl and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Carboxy” refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethenyl” refers to substituted or unsubstituted —(C═C)—.

“Ethylene” refers to substituted or unsubstituted —(C—C)—.

“Ethynyl” refers to —(C≡C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa}), —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, NR^{bb}P(═O)(NR^{bb})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═N^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{ee}, —P(═O)₂R^{cc}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, SC(═S)SC₁₋₆ alkyl, —P(═O)₂(C₁₋₆ alkyl), —P(═O)(C₁₋₆ alkyl)₂, —OP(═O)(C₁₋₆ alkyl)₂, OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻²sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR″, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and claims. The invention is not intended to be limited in any manner by the above exemplary listing of substituents.

### Other Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Pharmaceutical Compositions

In another aspect, the invention provides a pharmaceutical composition comprising a pharmaceutically acceptable carrier and a effective amount of a compound of Formulaes (I), (II-a), (II-b), (III), (IV), (V), (VI), or (VII).

When employed as pharmaceuticals, the compounds provided herein are typically administered in the form of a pharmaceutical composition. Such compositions can be prepared in a manner well known in the pharmaceutical art and comprise at least one active compound.

In one embodiment, with respect to the pharmaceutical composition, the carrier is a parenteral carrier, oral or topical carrier.

The present invention also relates to a compound of the present invention or pharmaceutical composition thereof for use as a pharmaceutical or a medicament.

Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herein can be administered by a variety of routes including oral, rectal, transdermal, subcutaneous, intravenous, intramuscular, and intranasal. Depending on the intended route of delivery, the compounds provided herein are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of this invention can also be administered in sustained release forms or from sustained release drug delivery systems. A description of representative sustained release materials can be found in Remington's Pharmaceutical Sciences.

The present invention also relates to the pharmaceutically acceptable formulations of a compound of the present invention. In one embodiment, the formulation comprises water. In another embodiment, the formulation comprises a cyclodextrin derivative. The most common cyclodextrins are α-, β- and γ-cyclodextrins consisting of 6, 7 and 8α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol®. See, e.g., U.S. Pat. No. 5,376,645. In certain embodiments, the formulation comprises hexapropyl-β-cyclodextrin. In a more particular embodiment, the formulation comprises hexapropyl-β-cyclodextrin (10-50% in water).

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

### Combination Treatment

In some embodiments, the methods described herein is used in combination with another method, such as a method of treatment comprising administering to a subject an additional therapeutic agent. Additional therapeutic agents are described herein. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

### Additional Therapies

Additional therapies include, but are not limited to dietary cholesterol therapy (e.g., cholesterol supplementation), statin treatment (e.g., 3-hydroxy-3-methylglutaryl coenzyme A reductase inhibitors (e.g., HMG CoA reductase inhibitors), bile acid supplementation or downstream hormone supplementation, medical therapies, and surgical interventions, antioxidants, and gene therapy.

### Statins

Statins are hydroxy-3-methylglutaryl coenzyme A (HMG-CoA) reductase inhibitors that inhibit the enzyme HMG-CoA reductase (the cholesterol pathway proximal to the enzymatic defect in SLOS). Exemplary statins include, but are not limited to, atorvastatin, fluvastatin, lovastatin, pitavastatin, pravastatin, rosuvastatin, and simvastatin.

1.-25. (canceled)

26. A method of treating Smith-Lemli-Opitz syndrome, comprising administering to a subject in need thereof a therapeutically effective amount of a compound selected from a group consisting of: 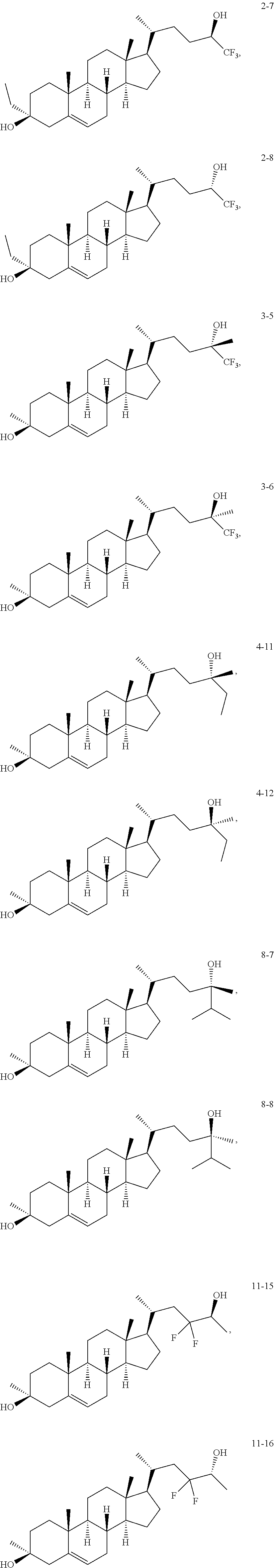
and pharmaceutically acceptable salts thereof.

27. The method of claim 26, wherein the compound is:

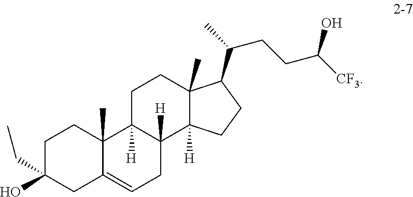

28. The method of claim 26, wherein the compound is:

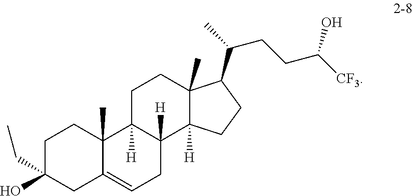

29. The method of claim 26, wherein the compound is:

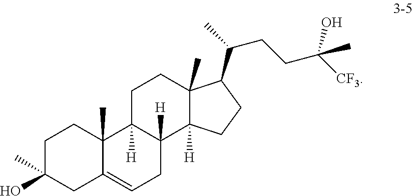

30. The method of claim 26, wherein the compound is:

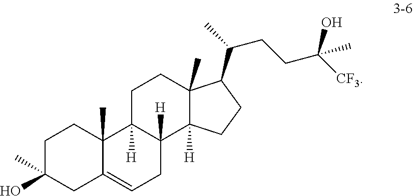

31. The method of claim 26, wherein the compound is:

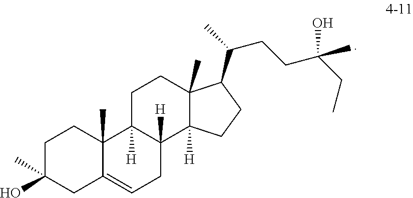

32. The method of claim 26, wherein the compound is:

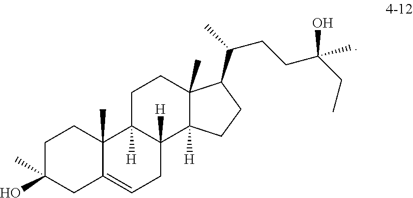

33. The method of claim 26, wherein the compound is:

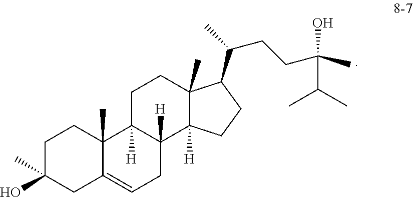

34. The method of claim 26, wherein the compound is:

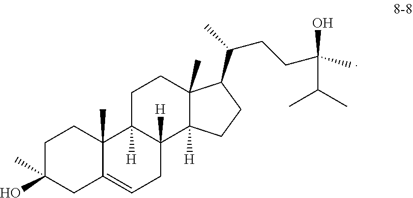

35. The method of claim 26, wherein the compound is:

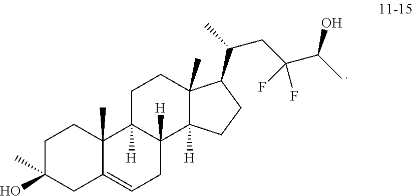

36. The method of claim 26, wherein the compound is:

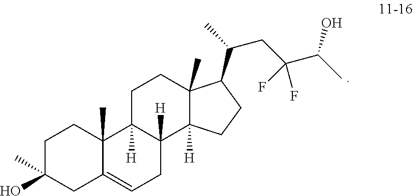

37. The method of claim 26, wherein the compound is a pharmaceutically acceptable salt of:

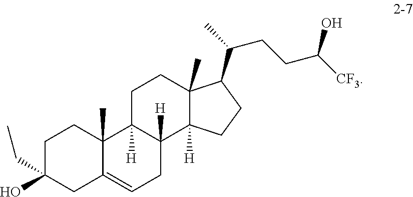

38. The method of claim 26, wherein the compound is a pharmaceutically acceptable salt of:

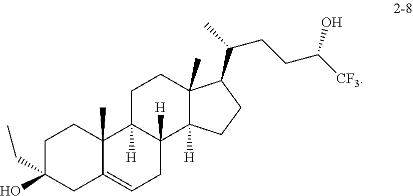

39. The method of claim 26, wherein the compound is a pharmaceutically acceptable salt of:

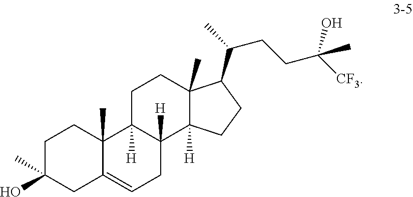

40. The method of claim 26, wherein the compound is a pharmaceutically acceptable salt of:

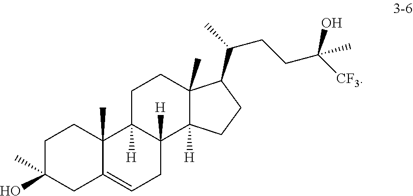

41. The method of claim 26, wherein the compound is a pharmaceutically acceptable salt of:

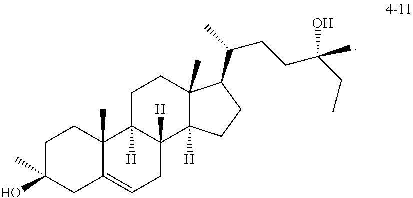

42. The method of claim 26, wherein the compound is a pharmaceutically acceptable salt of:

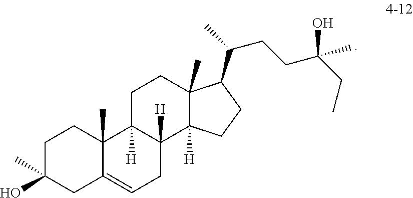

43. The method of claim 26, wherein the compound is a pharmaceutically acceptable salt of:

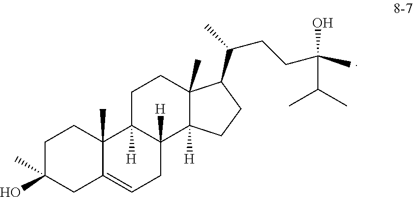

44. The method of claim 26, wherein the compound is a pharmaceutically acceptable salt of:

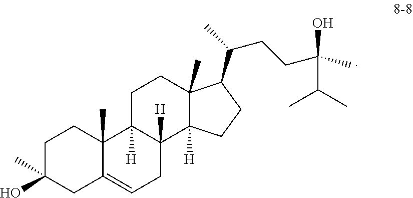

45. The method of claim 26, wherein the compound is a pharmaceutically acceptable salt of:

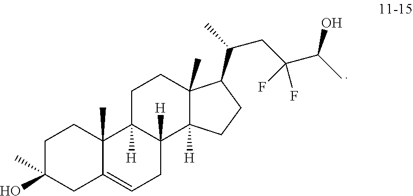

46. The method of claim 26, wherein the compound is a pharmaceutically acceptable salt of:

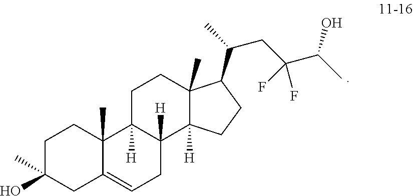

47. The method according to claim 26, the subject is an infant.

48. The method according to claim 26, wherein the subject is less than 21 years old.
